(19) United States
(12) Patent Application Publication
(10) Pub. No.: US 2025/0002941 A1
(43) Pub. Date: Jan. 2, 2025

(54) A BACTERIA-DERIVED LIPID COMPOSITION AND USE THEREOF

(71) Applicant: Sail Biomedicines, Inc., Cambridge, MA (US)

(72) Inventors: Siddharth PATEL, Cambridge, MA (US); Alaina HOWE, Cambridge, MA (US); Xiaoxue ZHANG, Cambridge, MA (US)

(21) Appl. No.: 18/712,576

(22) PCT Filed: Nov. 21, 2022
(86) PCT No.: PCT/US22/50571

§ 371 Date: May 22, 2024

Related U.S. Application Data
(60) Provisional application No. 63/282,304, filed on Nov. 23, 2021.

(51) Int. Cl. C12N 15/88; C12N 1/20

(52) U.S. Cl. CPC C12N 15/88; C12N 1/20

(57) ABSTRACT

Disclosed herein are bacteria-derived lipid compositions comprising (a) a bacterial component comprising one or more lipids extracted from a bacterial source; and (b) an ionizable lipid. The disclosure also includes a method for making a making a bacteria-derived lipid composition, comprising reconstructing (a) a bacteria component comprising one or more lipids extracted from a bacterial source in the presence of (b) an ionizable lipid, to produce the bacteria-derived lipid composition, and loading into the bacteria-derived lipid composition with one or more heterologous functional agents.

### CROSS-REFERENCE TO RELATED APPLICATIONS

This application claims benefit of priority to U.S. Provisional Application No. 63/282,304, filed Nov. 23, 2021, which is herein incorporated by reference in its entirety.

### BACKGROUND

The delivery of a heterologous functional agent (such as a therapeutic agent or immunologic agent) can be limited by the degree to which the agent can penetrate cell barriers and thereby effectively act on an organism. Therefore, there is a continuing need in the art for developing novel delivery systems that can effectively deliver the heterologous functional agent and promote cellular uptake of the agent.

### SUMMARY OF THE INVENTION

In one aspect, provided herein is a bacteria-derived lipid composition, comprising (a) a bacterial component comprising one or more lipids extracted from a bacterial source; and (b) an ionizable lipid.

In another aspect, provided herein is a bacteria-derived lipid composition comprising a plurality of lipid reconstructed bacterial components, wherein the lipid reconstructed bacterial components are produced by a process comprising the steps of (a) providing a plurality of purified bacterial lipids; (b) processing the plurality of purified bacterial lipids to produce a lipid film; (c) reconstituting the lipid film in an organic solvent selected from the group consisting of acetonitrile, acetone, ethanol, methanol, dimethylformamide, tetrahydrofuran, 1-buthanol, dimethyl sulfoxide, acetonitrile: ethanol, acetonitrile: methanol, acetone: methanol, methyl tert-butyl ether: propanol, tetrahydrofuran: methanol, dimethyl sulfoxide: methanol, and dimethylformamide: methanol, thereby producing a lipid solution; and (d) processing the lipid solution of step (c) in a microfluidics device comprising an aqueous phase, thereby producing the bacteria-derived lipid composition.

In another aspect, provided herein is a method for making a bacteria-derived lipid composition. The method comprises reconstructing (a) a bacteria component comprising one or more lipids extracted from a bacterial source in the presence of (b) an ionizable lipid, to produce the bacteria-derived lipid composition. The method further comprises loading into the bacteria-derived lipid composition with one or more heterologous functional agents. The ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

In some alternative embodiments, the ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 3.

In another aspect, provided herein is a method for delivering a bacteria-derived lipid composition to a target cell, the method comprising introducing a bacteria-derived lipid composition that comprises (a) a bacterial component comprising one or more lipids extracted from a bacterial source; and (b) an ionizable lipid to the target cell.

In some embodiments, the reconstructing step comprises reconstituting a film comprising the purified bacterial lipids of the bacteria component (a) in the presence of the ionizable lipid (b) to produce the bacteria-derived lipid composition.

In some embodiments, the bacterial source is selected from Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, and Thermoanaerobacterium. In one embodiment, the bacterial source is Escherichia (e.g., E. coli). In one embodiment, the bacterial source is Salmonella (e.g., Salmonella typhimurium).

In some embodiments, the bacterial component comprises isolated bacterial extracellular vesicles.

In some embodiments, the bacterial component is modified by reconstructing a film comprising the bacterial component in the presence of the ionizable lipid.

In some embodiments, the bacterial component is modified by reconstructing a film comprising the purified bacteria lipids of the bacterial component with the ionizable lipid.

In some embodiments, the ionizable lipid has one or more characteristics selected from the group consisting of:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

In some alternative embodiments, the ionizable lipid has one or more of the characteristics selected from the group consisting of:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 3.

In some embodiments, the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315. In one embodiment, the ionizable lipid is C12-200.

In some embodiments, the ionizable lipid is

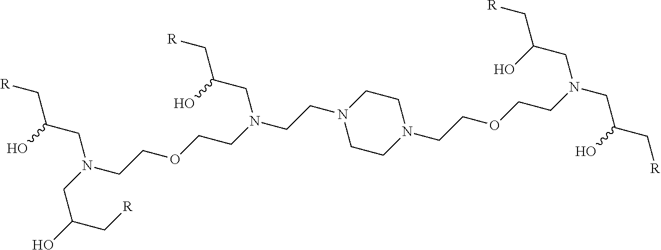

wherein R is a C₈-C₁₄ alkyl group.

In some embodiments, the bacteria-derived lipid composition further comprises a sterol. The reconstruction (or reconstitution) of the bacteria component is therefore carried out in the presence of an ionizable lipid and a sterol.

In some embodiments, the bacteria-derived lipid composition further comprises a polyethylene glycol (PEG)-lipid conjugate. The reconstruction (or reconstitution) of the bacteria component is therefore carried out in the presence of an ionizable lipid and a PEGylated lipid (or a PEG-lipid conjugate).

In some embodiments, the bacteria-derived lipid composition further comprises a sterol and a polyethylene glycol (PEG)-lipid conjugate. The reconstruction (or reconstitution) of the bacteria component is therefore carried out in the presence of an ionizable lipid, a sterol, and a PEGylated lipid (or a PEG-lipid conjugate).

In some embodiments, the sterol is cholesterol or sitosterol.

In some embodiments, the PEG-lipid conjugate is C14-PEG2k, C18-PEG2k, or DMPE-PEG2k. In some embodiments, the PEG-lipid conjugate is PEG-DMG or PEG-PE. In some embodiments, the PEG-DMG is PEG2000-DMG or PEG2000-PE.

In some embodiments, the bacteria-derived lipid composition comprises:
about 20 mol % to about 50 mol % of the ionizable lipid,
about 20 mol % to about 60 mol % of the bacterial component (e.g., the extracted bacterial lipids),
about 7 mol % to about 45 mol % of the sterol, and
about 0.5 mol % to about 3 mol % of the polyethylene glycol (PEG)-lipid conjugate.

In some embodiments, the bacteria-derived lipid composition comprises:
about 30 mol % to about 40 mol % of the ionizable lipid,
about 20 mol % to about 50 mol % of the bacterial component (e.g., the extracted bacterial lipids),
about 12 mol % to about 43 mol % of the sterol, and
about 1.5 mol % to about 3 mol % of the polyethylene glycol (PEG)-lipid conjugate.

In some embodiments, the bacteria-derived lipid composition comprises:
about 35 mol % of the ionizable lipid,
about 50 mol % of the bacterial component (e.g., the extracted bacterial lipids),
about 12.5 mol % of the sterol, and
about 2.5 mol % the polyethylene glycol (PEG)-lipid conjugate.

In one embodiment, the bacteria-derived lipid composition comprises ionizable lipid:bacterial lipids:sterol:PEG-lipid at a molar ratio of about 35:50:12.5:2.5.

In one embodiment, the bacteria-derived lipid composition comprises ionizable lipid:bacterial lipids:sterol:PEG-lipid at a molar ratio of about 35:20:42.5:2.5.

In some embodiments, the bacteria-derived lipid composition comprises:
lipids extracted from Escherichia (e.g., E. coli) or Salmonella (e.g., Salmonella typhimurium),
C12-200,
cholesterol, and
DMPE-PEG2k.

In one embodiment, the bacteria-derived lipid composition comprises:
polar lipids extracted from E. coli,
C12-200,
cholesterol, and
DMPE-PEG2k. The bacteria-derived lipid composition may comprise C12-200: E. coli polar lipids:cholesterol:DMPE-PEG2k at a molar ratio of about 35:50:12.5:2.5, or about 35:20:42.5:2.5.

In some embodiments, the bacteria-derived lipid composition is a lipophilic moiety selected from the group consisting of a lipoplex, a liposome, a lipid nanoparticle, a polymer-based carrier, an exosome, a lamellar body, a micelle, and an emulsion. In one embodiment, the bacteria-derived lipid composition is a liposome selected from the group consisting of a cationic liposome, a nanoliposome, a proteoliposome, a unilamellar liposome, a multilamellar liposome, a ceramide-containing nanoliposome, and a multivesicular liposome.

In some embodiments, the bacteria-derived lipid composition is a lipid nanoparticle.

In some embodiments, the particles of the bacteria-derived lipid composition have a size of less than about 200 nm. In one embodiment, the particles of the bacteria-derived lipid composition have a size of less than about 150 nm. In one embodiment, the particles of the bacteria-derived lipid composition have a size of less than about 100 nm. In one embodiment, the particles of the bacteria-derived lipid composition have a size of about 55 nm to about 95 nm. In one embodiment, the particles of the bacteria-derived lipid composition have a size of about 85 nm to about 95 nm. In one embodiment, the particles of the bacteria-derived lipid composition have a size of about 85 nm to about 90 nm.

In some embodiments, the average polydispersity index (PDI) of the particles of the bacteria-derived lipid composition ranges from about 0.1 to about 0.5. In some embodiments, the average PDI of the particles of the bacteria-derived lipid composition ranges from about 0.1 to about 0.4. In some embodiments, the average PDI of the particles of the bacteria-derived lipid composition ranges from about 0.2 to about 0.3.

In some embodiments, the bacteria-derived lipid composition comprises one or more heterologous functional agents. In some embodiments, the heterologous functional agent is encapsulated by the bacteria-derived lipid composition. In some embodiments, the heterologous functional agent is embedded on the surface of the bacteria-derived lipid composition. In some embodiments, the heterologous functional agent is conjugated to the surface of the bacteria-derived lipid composition.

In some embodiments, the heterologous functional agent is a polynucleotide. In some embodiments, the polynucleotide is chosen from an mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In some embodiments, the polynucleotide is an mRNA. In some embodiments, the mRNA is derived from a DNA molecule or an RNA molecule (e.g., a self-replicating RNA molecule). In some embodiments, the polynucleotide is an siRNA or a precursor thereof. In some embodiments, the polynucleotide is a plasmid.

In some embodiments, the encapsulation efficiency of the polynucleotide by the bacteria-derived lipid composition is at least about 5%, 10%, 15%, 20%, 30%, 40%, 50%, 60%, 70%, 80%, 90%, 95%, 98%, 99%, or more than 99%. In one embodiment, the encapsulation efficiency of the polynucleotide by the bacteria-derived lipid composition is at least about 90%.

In some embodiments, the bacterial derived lipid nanoparticle has a total lipid:heterologous functional agent (e.g., polynucleotide) weight ratio of about 50:1 to about 10:1. In some embodiments, the bacterial derived lipid nanoparticle has a total lipid:heterologous functional agent (e.g., polynucleotide) weight ratio of about 44:1 to about 24:1. In some embodiments, the bacterial derived lipid nanoparticle has a total lipid:heterologous functional agent (e.g., polynucleotide) weight ratio of about 40:1 to about 28:1. In some embodiments, the bacterial derived lipid nanoparticle has a total lipid:heterologous functional agent (e.g., polynucleotide) weight ratio of about 38:1 to about 30:1. In some embodiments, the bacterial derived lipid nanoparticle has a total lipid:heterologous functional agent (e.g., polynucleotide) weight ratio of about 37:1 to about 33:1.

In some embodiments, the bacteria-derived lipid composition is formulated for delivery to an animal or a human. In some embodiments, the bacteria-derived lipid composition is formulated for delivery to a plant.

In some embodiments, the bacteria-derived lipid composition is produced by a method comprising lipid extrusion. In some embodiments, the bacteria-derived lipid composition is produced by a method comprising processing a solution comprising the lipids from the bacteria-derived lipid composition in a microfluidics device comprising an aqueous phase, thereby producing the bacteria-derived lipid composition.

In some embodiments, the heterologous functional agent is formulated into the bacteria-derived lipid composition via an aqueous phase. In some embodiments, the aqueous phase and the lipid solution (organic phase) are mixed at a 3:1 volumetric ratio.

In some embodiments, the aqueous phase comprises the polynucleotides.

In some embodiments, the bacteria-derived lipid composition, e.g., the aqueous phase, further comprises a HEPES or TRIS buffer. The HEPES or TRIS buffer may have a pH of about 7.0 to about 8.5. The HEPES or TRIS buffer can be at a concentration of about 7 mg/mL to about 15 mg/mL. The aqueous phase may further comprise about 2.0 mg/mL to about 4.0 mg/ml of NaCl.

In some embodiments, the bacteria-derived lipid composition, e.g., the aqueous phase comprises water, PBS, or a citrate buffer. In one embodiment, the aqueous phase comprises a citrate buffer having a pH of about 3.2.

In some embodiments, the bacteria-derived lipid composition further comprises one or more cryoprotectants. The one or more cryoprotectants may be sucrose, glycerol, or a combination thereof. In some embodiments, the bacteria-derived lipid composition comprises sucrose, e.g., at a concentration of about 70 mg/mL to about 110 mg/mL. In some embodiments, the bacteria-derived lipid composition comprises glycerol, e.g., at a concentration of about 50 mg/mL to about 70 mg/mL. In one embodiment, the bacteria-derived lipid composition comprises a combination of sucrose (e.g., at a concentration of about 70 mg/mL to about 110 mg/mL) and glycerol (e.g., at a concentration of about 50 mg/mL to about 70 mg/mL).

In some embodiments, the bacteria-derived lipid composition is a freeze-dried or lyophilized composition. The freeze-dried or lyophilized bacteria-derived lipid composition may comprise one or more lyoprotectants. The lyophilized bacteria-derived lipid composition may comprise a poloxamer, potassium sorbate, sucrose, or any combination thereof. In one embodiment, the lyophilized bacteria-derived lipid composition comprises a poloxamer (e.g., about 0.01 to about 1.0% w/w of a poloxamer) In one embodiment, the poloxamer is poloxamer 188.

In some embodiments, the bacteria-derived lipid composition is a lyophilized composition. In some embodiments, the lyophilized bacteria-derived lipid composition comprises about 0.01 to about 1.0% w/w of the heterologous functional agents (e.g., polynucleotides). In some embodiments, the lyophilized bacteria-derived lipid composition comprises about 1.0 to about 5.0% w/w lipids. In some embodiments, the lyophilized bacteria-derived lipid composition comprises about 0.5 to about 2.5% w/w of TRIS buffer. In some embodiments, the lyophilized bacteria-derived lipid composition comprises about 0.75 to about 2.75% w/w of NaCl. In some embodiments, the lyophilized bacteria-derived lipid composition comprises about 85 to about 95% w/w of a sugar, e.g., sucrose. In some embodiments, the lyophilized bacteria-derived lipid composition comprises about 0.01 to about 1.0% w/w of a poloxamer (e.g., about 0.01 to about 1.0% w/w of a poloxamer) such as poloxamer 188. In some embodiments, the lyophilized bacteria-derived lipid composition comprises about 1.0 to about 5.0% w/w of potassium sorbate.

### Definitions

As used herein, the term “effective amount,” “effective concentration,” or “concentration effective to” refers to an amount of a bacteria-derived lipid composition, or nucleic acid composition, sufficient to effect the recited result or to reach a target level (e.g., a predetermined or threshold level) in or on a target organism.

As used herein, the term “therapeutic agent” refers to an agent that can act on an animal, e.g., a mammal (e.g., a human), an animal pathogen, or a pathogen vector, such as an antifungal agent, an antibacterial agent, a virucidal agent, an anti-viral agent, an insecticidal agent, a nematicidal agent, an antiparasitic agent, or an insect repellent.

As used herein, the term “heterologous” refers to an agent that is exogenous to the organism that the bacteria-derived lipid composition is delivered to.

As used herein, the term “functional agent” refers to an agent (e.g., an agricultural agent (e.g., pesticidal agent, fertilizing agent, herbicidal agent, plant-modifying agent) or a therapeutic agent (e.g., an antifungal agent, an antibacterial agent, a virucidal agent, an anti-viral agent, an insecticidal agent, a nematicidal agent, an antiparasitic agent, or an insect repellent)) that is or can be associated with the bacteria-derived lipid composition (e.g., loaded into or onto the bacteria-derived lipid composition (e.g., encapsulated by, embedded in, or conjugated to the bacteria-derived lipid composition)) using in vivo or in vitro methods and is capable of effecting the recited result (e.g., increasing or decreasing the fitness of a plant, plant pest, plant symbiont, animal (e.g., human) pathogen, or animal pathogen vector). In some embodiments, the heterologous functional agent is a polynucleotide.

As defined herein, the term “nucleic acid” and “polynucleotide” are interchangeable and refer to RNA or DNA that is linear or branched, single or double stranded, or a hybrid thereof, regardless of length (e.g., at least 2, 3, 4, 5, 6, 7, 8, 9, 10, 20, 30, 40, 50, 100, 150, 200, 250, 500, 1000, or more nucleic acids). The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, the term “peptide,” “protein,” or “polypeptide” encompasses any chain of naturally or non-naturally occurring amino acids (either D- or L-amino acids), regardless of length (e.g., at least 2, 3, 4, 5, 6, 7, 10, 12, 14, 16, 18, 20, 25, 30, 40, 50, 100, or more amino acids), the presence or absence of post-translational modifications (e.g., glycosylation or phosphorylation), or the presence of, e.g., one or more non-amino acyl groups (for example, sugar, lipid, etc.) covalently linked to the peptide, and includes, for example, natural proteins, synthetic, or recombinant polypeptides and peptides, hybrid molecules, peptoids, or peptidomimetics.

As used herein, “percent identity” between two sequences is determined by the BLAST 2.0 algorithm, which is described in Altschul et al., (1990) J. Mol. Biol. 215:403-410. Software for performing BLAST analyses is publicly available through the National Center for Biotechnology Information.

As used herein, the term “unmodified bacterial component” refers to a composition including a bacterial component (e.g., isolated bacterial extracellular vesicles, or extracted bacterial lipids) that lack a heterologous cell uptake agent capable of increasing cell uptake (e.g., animal cell uptake, plant cell uptake, bacterial cell uptake, or fungal cell uptake) of the bacterial component.

As used herein, the term “modified” or “modification” to the bacterial component refers to a bacteria-derived lipid composition including a bacterial component and one or more heterologous agents (e.g., one or more exogenous lipids, such as an ionizable lipid, sterol and/or a PEGylated lipid) capable of increasing cell uptake (e.g., animal cell uptake, plant cell uptake, bacterial cell uptake, or fungal cell uptake) of the bacteria-derived lipid composition, or a portion or component thereof, relative to an unmodified bacterial component; capable of enabling or increasing delivery of a heterologous functional agent (e.g., an agricultural or therapeutic agent) by the bacteria-derived lipid composition to a cell, and/or capable of enabling or increasing loading (e.g., loading efficiency or loading capacity) of a heterologous functional agent (e.g., an agricultural or therapeutic agent). The bacterial component may be modified in vitro or in vivo.

As used herein, the term “cell uptake” refers to uptake of a bacteria-derived lipid composition or a portion or component thereof (e.g., a polynucleotide carried by the bacteria-derived lipid composition) by a cell, such as an animal cell, a plant cell, bacterial cell, or fungal cell. For example, uptake can involve transfer of the bacteria-derived lipid composition or a portion of component thereof from the extracellular environment into or across the cell membrane, the cell wall, the extracellular matrix, or into the intracellular environment of the cell). Cell uptake of bacteria-derived lipid composition may occur via active or passive cellular mechanisms. Cell uptake includes aspects in which the entire bacteria-derived lipid composition is taken up by a cell, e.g., taken up by endocytosis. In some embodiments, one or more polynucleotides are exposed to the cytoplasm of the target cell following endocytosis and endosomal escape. In some embodiments, a bacteria-derived lipid composition comprising an ionizable lipid, e.g., a bacteria-derived lipid composition comprising an ionizable lipid and a sterol and/or a PEGylated lipid) has an increased rate of endosomal escape relative to an unmodified bacterial component (e.g., extracted bacterial lipids). Cell uptake also includes aspects in which the bacteria-derived lipid composition fuses with the membrane of the target cell. In some embodiments, one or more polynucleotides are exposed to the cytoplasm of the target cell following membrane fusion. In some embodiments, a bacteria-derived lipid composition has an increased rate of fusion with the membrane of the target cell (e.g., is more fusogenic) relative to a bacterial component that is not modified by an ionizable lipid.

As used herein, the term “cell-penetrating agent” refers to agents that alter properties (e.g., permeability) of the cell wall, extracellular matrix, or cell membrane of a cell (e.g., an animal cell, a plant cell, a bacterial cell, or a fungal cell) in a manner that promotes increased cell uptake relative to a cell that has not been contacted with the agent.

A bacteria-derived lipid composition comprising a lipid reconstructed bacterial component is described herein. The bacterial component has been derived from a lipid structure (e.g., a lipid bilayer, unilamellar, multilamellar structure; e.g., a vesicular lipid structure) derived from (e.g., enriched, isolated or purified from) a bacterial source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the lipid reconstructed bacterial component, as is described herein. The method may, if desired, further comprise sonication, freeze/thaw treatment, and/or lipid extrusion, e.g., to reduce the size of the reconstituted bacteria-derived lipid composition. Alternatively, the bacteria-derived lipid composition may be produced using a microfluidic device (such as a NanoAssemblr® IGNITE™ microfluidic instrument (Precision NanoSystems)).

As used herein, the term “bacterial extracellular vesicle”, “bacterial EV”, or “EV” refers to an enclosed lipid-bilayer structure naturally occurring in a bacterial. Optionally, the bacterial EV includes one or more bacterial EV markers. As used herein, the term “bacterial EV marker” refers to a component that is naturally associated with a bacterial, such as a bacterial protein, a bacterial nucleic acid, a bacterial small molecule, a bacterial lipid, or a combination thereof.

As used herein, the term “cationic lipid” refers to an amphiphilic molecule (e.g., a lipid or a lipidoid) that is positively charged, containing a cationic group (e.g., a cationic head group).

As used herein, the term “ionizable lipid” refers to an amphiphilic molecule (e.g., a lipid or a lipidoid, e.g., a synthetic lipid or lipidoid) containing a group (e.g., a head group) that can be ionized, e.g., dissociated to produce one or more electrically charged species, under a given condition (e.g., pH).

It has been surprisingly found that ionizable lipids comprising alkyl chains with multiple sites of unsaturation, e.g., at least two or three sites of unsaturation, are particularly useful for forming lipid particles with increased membrane fluidity. A number of ionizable lipids and related analogs, suitable for use herein, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes.

In some embodiments, ionizable lipids are ionizable such that they can dissociate to exist in a positively charged form depending on pH. The ionization of an ionizable lipid affects the surface charge of a lipid nanoparticle comprising the ionizable lipid under different pH conditions. The surface charge of the lipid nanoparticle in turn can influence its plasma protein absorption, blood clearance, and tissue distribution (Semple, S. C., et al., Adv. Drug Deliv Rev 32:3-17 (1998)) as well as its ability to form endosomolytic non-bilayer structures (Hafez, I. M., et al., Gene Ther 8:1188-1196 (2001)) that can influence the intracellular delivery of nucleic acids.

In some embodiments, ionizable lipids are those that are generally neutral, e.g., at physiological pH (e.g., pH about 7), but can carry net charge(s) at an acidic pH or basic pH. In one embodiment, ionizable lipids are those that are generally neutral at pH about 7, but can carry net charge(s) at an acidic pH. In one embodiment, ionizable lipids are those that are generally neutral at pH about 7, but can carry net charge(s) at a basic pH.

In some embodiments, ionizable lipids do not include those cationic lipids or anionic lipids that generally carry net charge(s) at physiological pH (e.g., pH about 7).

As used herein, the term “lipidoid” refers to a molecule having one or more characteristics of a lipid.

As used herein, the term “stable bacteria-derived lipid formulation” refers to a bacteria-derived lipid composition that over a period of time (e.g., at least 24 hours, at least 48 hours, at least 1 week, at least 2 weeks, at least 3 weeks, at least 4 weeks, at least 30 days, at least 60 days, or at least 90 days) retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of the initial number of the particles of the bacteria-derived lipid composition (e.g., particles per mL of solution) relative to the number of the particles in the bacteria-derived lipid formulation (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)); or retains at least 5% (e.g., at least 5%, 10%, 15%, 20%, 25%, 30%, 35%, 40%, 45%, 50%, 55%, 60%, 65%, 70%, 75%, 80%, 85%, 90%, 95%, or 100%) of its activity (e.g., cell wall penetrating activity and/or activity of the mRNA formulated within the bacteria-derived lipid composition) relative to the initial activity of the bacteria-derived lipid composition (e.g., at the time of production or formulation) optionally at a defined temperature range (e.g., a temperature of at least 24° C. (e.g., at least 24° C., 25° C., 26° C., 27° C., 28° C., 29° C., or 30° C.), at least 20° C. (e.g., at least 20° C., 21° C., 22° C., or 23° C.), at least 4° C. (e.g., at least 5° C., 10° C., or 15° C.), at least −20° C. (e.g., at least −20° C., −15° C., −10° C., −5° C., or 0° C.), or −80° C. (e.g., at least −80° C., −70° C., −60° C., −50° C., −40° C., or −30° C.)).

As used herein, the term “formulated for delivery to an animal” refers to a bacteria-derived lipid composition that includes a pharmaceutically acceptable carrier. As used herein, a “pharmaceutically acceptable” carrier or excipient is one that is suitable for administration to an animal (e.g., human), e.g., without undue adverse side effects to the animal (e.g., human).

### BRIEF DESCRIPTION OF THE DRAWINGS

FIG. 1 is a scheme showing a workflow for preparation of bacteria-derived lipid composition by modifying extracted bacterial lipids with an ionizable lipid.

FIG. 2 shows the molar ratio of various components constituting an exemplary bacteria-derived lipid composition (BacLC), as compared to the molar ratios of various components constituting a conventional lipid nanoparticle composition (LNP), as described in Examples 2 and 5.

FIG. 3A shows the size and polydispersity of the particles of an exemplary bacteria-derived lipid composition (E. coli BacLC), as compared to those of conventional lipid nanoparticle composition (LNP), prepared according to Examples 2 and 5. FIG. 3B shows the encapsulation efficiency of the particles of the E. coli BacLC/mRNA formulation, as compared to those of the comparative formulation (LNP/mRNA), prepared according to Example 5.

FIG. 4 shows the number of antigen-specific T cells producing IFNg in 100 μL blood of the mice at 12 days, after a single dose intramuscular delivery of BacLC E. coli (Avanti)/SARS-CoV-2 (E. coli (Avanti) BacLC, containing S mRNA 1 μg) in the mice, as compared to the mice having intramuscular delivery of a comparative formulation (LNP/mRNA, containing S mRNA 1 μg), prepared according to Example 5. Control was PBS.

FIG. 5 shows the levels of antibody (IgG) specific to S1 antigen of SARS-CoV-2 in the plasma of the mice at 12 days, after a single dose intramuscular delivery of BacLC E. coli (Avanti)/SARS-CoV-2 (E. coli (Avanti) BacLC, containing S mRNA 1 μg) in the mice, as compared to the mice having intramuscular delivery of a comparative formulation (LNP/mRNA, containing S mRNA 1 μg), prepared according to Example 5. Control was PBS.

FIG. 6 shows the number of SARS-CoV-2 S-specific T cells producing cytokine IFNg per 10⁶ splenocytes in mice at 28 days, after a single dose intramuscular delivery of BacLC E. coli (Avanti)/SARS-CoV-2 (E. coli (Avanti) BacLC, containing S mRNA 1 μg) in the mice, as compared to the mice having intramuscular delivery of a comparative formulation (LNP/mRNA, containing S mRNA 1 μg), prepared according to Example 5. Control was PBS.

FIG. 7 shows the number of SARS-CoV-2 S-specific T cells producing cytokine IFNg per 10⁶ splenocytes in the mice at 12 days after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS.

FIG. 8 shows the levels of antibody (IgG) specific to the receptor binding domain (RBD) of SARS-CoV-2 in the plasma of the mice at 12 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS.

FIG. 9 shows the levels of antibody (IgG) specific to the receptor binding domain (RBD) of SARS-CoV-2 in the plasma of the mice at 28 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS.

FIGS. 10A-10B show the levels of antibody (IgA) specific to the receptor binding domain (RBD) (FIG. 10A) or S-protein (FIG. 10B), respectively, of SARS-CoV-2 in the plasma of the mice at 28 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS.

FIG. 10C-10D show the levels of antibody (IgA) specific to the receptor binding domain (RBD) (FIG. 10C) or S-protein (FIG. 10D), respectively, of SARS-CoV-2 in the bronchoalveolar lavage fluid (BALF) of the mice at 28 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS.

FIG. 11 shows the number of influenza HA-specific T cells producing cytokine IFNg per 10⁶ splenocytes in the mice at 14 days after a single dose intramuscular delivery of BacLC E. coli (Avanti)/Influenza (E. coli (Avanti) BacLC, containing HA mRNA 10 μg), as compared to the mice having intramuscular delivery of the comparative formulation (LNP) prepared according to Example 5. Control was PBS.

FIG. 12 shows the levels of antibody (IgG) specific to the hemagglutinin (HA) of influenza in the plasma of the mice at 14 days, after a single dose intramuscular delivery of BacLC E. coli (Avanti)/Influenza (E. coli (Avanti) BacLC, containing HA mRNA 10 μg), as compared to the mice having intramuscular delivery of the comparative formulation (LNP) prepared according to Example 5. Control was PBS.

FIGS. 13A-13C show the frequency of tdTomato+ lymphocytes (FIG. 13A), myeloid cells (FIG. 13B), and non-immune cells (FIG. 13C), respectively, in the spleens of Ai9 mice at 6 days after a single dose intramuscular delivery of BacLC E. coli (Avanti)/mRNA (E. coli (Avanti) BacLC, containing CRE mRNA 10 μg), as compared to the mice having intramuscular delivery of the comparative formulation (LNP) prepared according to Example 5. Control was PBS.

FIGS. 14A-14C show the frequency of tdTomato+ lymphocytes (FIG. 14A), myeloid cells (FIG. 14B), and non-immune cells (FIG. 14C), respectively, in the lymph nodes of Ai9 mice at 6 days after a single dose intramuscular delivery of BacLC E. coli (Avanti)/mRNA (E. coli (Avanti) BacLC, containing CRE mRNA 10 μg), as compared to the mice having intramuscular delivery of the comparative formulation (LNP) prepared according to Example 5. Control was PBS.

FIGS. 15A-C show the whole body (FIG. 15A), liver (FIG. 15B), and spleen (FIG. 15C) radiance of the mice 4-6 hours after the mice were intravenously administered with a dosage of BacLC E. coli/mRNA (E. coli BacLC, containing FLuc:EPO mRNA 10 μg), BacLC E. coli (Avanti)/mRNA (E. coli (Avanti) BacLC, containing FLuc:EPO mRNA 10 μg), and BacLC Salmonella/mRNA (Salmonella BacLC, containing FLuc:EPO mRNA 10 μg), prepared according to Example 5. FIG. 15D shows the spleen to liver ratio of radiance 4-6 hours after the mice were intravenously administered with a dosage of BacLC E. coli/mRNA (E. coli BacLC, containing FLuc:EPO mRNA 10 μg), BacLC E. coli (Avanti)/mRNA (E. coli (Avanti) BacLC, containing FLuc:EPO mRNA 10 μg), and BacLC Salmonella/mRNA (Salmonella BacLC, containing FLuc:EPO mRNA 10 μg).

FIGS. 16A-C show the mesenteric lymph node (FIG. 16A), stomach (FIG. 16B), and mesenteric fat pad (FIG. 16D) radiance of the mice 4-6 hours after the mice were intravenously administered with a dosage of BacLC E. coli/mRNA (E. coli BacLC, containing FLuc:EPO mRNA 10 μg), BacLC E. coli (Avanti)/mRNA (E. coli (Avanti) BacLC, containing FLuc:EPO mRNA 10 μg), and BacLC Salmonella/mRNA (Salmonella BacLC, containing FLuc:EPO mRNA 10 μg), prepared according to Example 5.

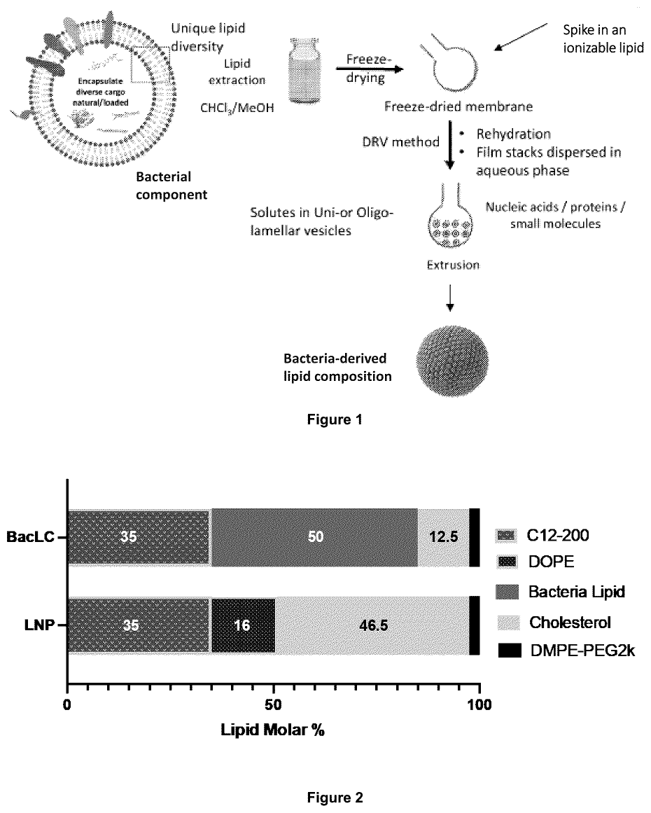

### DETAILED DESCRIPTION

Featured herein are bacteria-derived lipid compositions that contain (a) a bacterial component comprising one or more lipids extracted from a bacterial source; and (b) an ionizable lipid that can increase cell uptake of the bacterial component. These bacteria-derived lipid compositions may be formulated with one or more heterologous functional agents, such as polynucleotides, and can be used as delivering vehicles for these heterologous functional agents (e.g., polynucleotides).

### Bacterial Component

The bacterial component comprises one or more lipids extracted from a bacterial source.

The bacterial component may have a lipid (e.g., lipid bilayer, unilamellar, or multilamellar structure) structure that includes a bacterial extracellular vesicle (EV), or segment, portion, or extract (e.g., lipid extract) thereof.

The bacterial component comprises isolated bacterial extracellular vesicles. Bacterial EVs refer to an enclosed lipid-bilayer structure that naturally occurs in a bacterium. Bacterial EVs are derived from a bacterium that comprise bacterial lipids, and may also comprise bacterial proteins and/or bacterial nucleic acids and/or carbohydrate moieties contained in a nanoparticle. The bacterial EVs may contain 1, 2, 3, 4, 5, 10, or more than 10 different lipid species.

As used herein, the term “bacteria” broadly refers to the domain of prokaryotic organisms, including Gram positive and Gram negative organisms. Suitable bacterial sources include Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, and Thermoanaerobacterium.

In one embodiment, the bacterial source is Escherichia (e.g., E. coli). In one embodiment, the bacterial source is Salmonella (e.g., Salmonella typhimurium).

In some embodiments, the bacterial source belongs to Actinobacteria or Proteobacteria, such as in the families of the Burkholderiaceae, Xanthomonadaceae, Pseudomonadaceae, Enterobacteriaceae, Microbacteriaceae, and Rhizobiaceae.

In some embodiments, the bacterial source is an Agrobacterium spp., Sinorhizobium (=Ensifer) sp., Mesorhizobium, sp., Bradyrhizobium sp., Azobacter sp., Phyllobacter sp.), Sinorhizobium (=Ensifer) sp., Mesorhizobium, sp., Azorhizobium sp., Bradyrhizobium sp., or Rhizobium sp.

In some embodiments, the bacterial source is an Acidovorax avenae subsp., Burkholderia spp., Liberibacter spp., Corynebacterium spp., Erwinia spp., Pseudomonas syringae subsp., Streptomyces spp., Xanthomonas axonopodis subsp., Xanthomonas campestris pv. musacearum, Xanthomonas campestris pv. pruni (=Xanthomonas arboricola pv. pruni), Xanthomonas fragariae., Xanthomonas translucens supsp. (=Xanthomonas campestris pv. hordei), Xanthomonas oryzae supsp., Xanthomonas oryzae pv. oryzae (=Xanthomonas campestris pv. oryzae), or Xanthomonas oryzae pv. oryzicola (=Xanthomonas campestris pv. oryzicola).

Additional examples of species and/or strains of bacteria that can be used include those listed in Tables 1-2 of U.S. Patent Application Publication No. 2020/0254028, which is incorporated herein by reference in its entirety.

The bacterial component can include bacterial EVs, or segments, portions, or extracts, thereof. In some embodiments, the bacterial EVs are about 5-1000 nm in diameter. For example, the bacterial component can include a bacterial EV, or segment, portion, or extract thereof, that has a mean diameter of about 5-50 nm, about 50-100 nm, about 100-150 nm, about 150-200 nm, about 200-250 nm, about 250-300 nm, about 300-350 nm, about 350-400 nm, about 400-450 nm, about 450-500 nm, about 500-550 nm, about 550-600 nm, about 600-650 nm, about 650-700 nm, about 700-750 nm, about 750-800 nm, about 800-850 nm, about 850-900 nm, about 900-950 nm, about 950-1000 nm, about 1000-1250 nm, about 1250-1500 nm, about 1500-1750 nm, or about 1750-2000 nm. In some instances, the bacterial component can include a bacterial EV, or segment, portion, or extract thereof, that has a mean diameter of about 5-950 nm, about 5-900 nm, about 5-850 nm, about 5-800 nm, about 5-750 nm, about 5-700 nm, about 5-650 nm, about 5-600 nm, about 5-550 nm, about 5-500 nm, about 5-450 nm, about 5-400 nm, about 5-350 nm, about 5-300 nm, about 5-250 nm, about 5-200 nm, about 5-150 nm, about 5-100 nm, about 5-50 nm, or about 5-25 nm. In certain instances, the bacterial component can include a bacterial EV, or segment, portion, or extract thereof, having a mean diameter of about 50-200 nm, about 50-300 nm, about 200-500 nm, or about 30-150 nm. A variety of methods (e.g., a dynamic light scattering method) standard in the art can be used to measure the particle diameter of the bacterial EV, or segment, portion, or extract thereof.

In some instances, the bacterial component can include a bacterial EV, or segment, portion, or extract thereof, that has a mean surface area of at least 77 nm², (e.g., at least 77 nm², at least 100 nm², at least 1000 nm², at least 1×10⁴ nm², at least 1×10⁵ nm², at least 1×10⁶ nm², or at least 2×10⁶ nm²). In some instances, the bacterial component can include a bacterial EV, or segment, portion, or extract thereof, that has a mean surface area of 77 nm² to 3.2×10⁶ nm² (e.g., 77-100 nm², 100-1000 nm², 1000-1×10⁴ nm², 1×10⁴-1×10⁵ nm², 1×10⁵-1×10⁶ nm², or 1×10⁶-3.2×10⁶ nm²).

In some instances, the bacterial component can include a bacterial EV, or segment, portion, or extract thereof, that has a mean volume of at least 65 nm³ (e.g., at least 65 nm³, at least 100 nm³, at least 1000 nm³, at least 1×10⁴ nm³, at least 1×10⁵ nm³, at least 1×10⁶ nm³, at least 1×10⁷ nm³, at least 1×10⁸ nm³, at least 2×10⁸ nm³, at least 3×10⁸ nm³, at least 4×10⁸ nm³, or at least 5×10⁸ nm³. In some instances, the bacterial component can include a bacterial EV, or segment, portion, or extract thereof, that has a mean volume of 65 nm³ to 5.3×10⁸ nm³ (e.g., 65-100 nm³, 100-1000 nm³, 1000-1×10⁴ nm³, 1×10⁴-1×10⁵ nm³, 1×10⁵-1×10⁶ nm³, 1×10⁶-1×10⁷ nm³, 1×10⁷-1×10⁸ nm³, 1×10⁸-5.3×10⁸ nm³).

In some instances, the bacterial component may include an intact bacterial EV. Alternatively, the bacterial component may include a segment, portion, or extract of the full surface area of the vesicle (e.g., a segment, portion, or extract including less than 100% (e.g., less than 90%, less than 80%, less than 70%, less than 60%, less than 50%, less than 40%, less than 30%, less than 20%, less than 10%, less than 10%, less than 5%, or less than 1%) of the full surface area of the vesicle) of a bacterial EV. The segment, portion, or extract may be any shape, such as a circumferential segment, spherical segment (e.g., hemisphere), curvilinear segment, linear segment, or flat segment. In instances where the segment is a spherical segment of the vesicle, the spherical segment may represent one that arises from the splitting of a spherical vesicle along a pair of parallel lines, or one that arises from the splitting of a spherical vesicle along a pair of non-parallel lines. Accordingly, the bacterial component can include a plurality of intact bacterial EVs, a plurality of bacterial EV segments, portions, or extracts, or a mixture of intact and segments of EVs. One skilled in the art will appreciate that the ratio of intact to segmented bacterial EVs will depend on the particular isolation method used. For example, grinding or blending a bacterial, or part thereof, may produce the bacterial component that contain a higher percentage of bacterial EV segments, portions, or extracts than a non-destructive extraction method, such as vacuum-infiltration.

In instances where, the bacterial component includes a segment, portion, or extract of a bacterial EV, the EV segment, portion, or extract may have a mean surface area less than that of an intact vesicle, e.g., a mean surface area less than 77 nm², 100 nm², 1000 nm², 1×10⁴ nm², 1×10⁵ nm², 1×10⁶ nm², or 3.2×10⁶ nm²). In some instances, the bacterial component may include a bacterial EV, or segment, portion, or extract thereof, that has a mean volume less than that of an intact vesicle, e.g., a mean volume of less than 65 nm³, 100 nm³, 1000 nm³, 1×10⁴ nm³, 1×10⁵ nm³, 1×10⁶ nm³, 1×10⁷ nm³, 1×10⁸ nm³, or 5.3×10⁸ nm³).

The bacterial component includes one or more lipids extracted from a bacterial source. In some embodiments, the bacterial component may include at least 1%, 2%, 5%, 10%, 20%, 30%, 40%, 50%, 60%, 70%, 80%, 90%, 95%, 99%, or more than 99%, of lipids extracted a bacterial source. The bacterial component may include bacterial EV segments and/or extracted lipids or a mixture thereof.

### Production of the Bacterial Component

The bacterial component may be produced from bacterial EVs, or a segment, portion or extract (e.g., lipid extract) thereof, that occur naturally in a bacterium. An exemplary method for producing a bacterial component includes (a) providing an initial sample from a bacterium; and (b) isolating a crude bacterial fraction from the initial sample, wherein the crude bacterial fraction has a decreased level of at least one contaminant or undesired component from the bacterium relative to the level in the initial sample. The method can further include an additional step (c) comprising purifying the crude bacterial fraction, thereby producing a pure bacterial component, having a decreased level of at least one contaminant or undesired component from the bacterium relative to the level in the crude EV fraction.

In some instances, the bacterial component may be isolated from a bacterial source by a process which includes the steps of: (a) providing an initial sample from a bacterium; (b) isolating a crude bacterial fraction from the initial sample, wherein the crude bacterial fraction has a decreased level of at least one contaminant or undesired component from the bacterium relative to the level in the initial sample (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%); and (c) purifying the crude bacterial fraction, thereby producing a pure bacterial component having a decreased level of at least one contaminant or undesired component from the bacterium relative to the level in the crude EV fraction (e.g., a level that is decreased by at least 1%, 2%, 5%, 10%, 15%, 20%, 25%, 30%, 40%, 45%, 50%, 55%, 60%, 70%, 80%, 90%, 95%, 96%, 98%, 99%, or 100%).

The bacterial component (e.g., the bacterial lipids) may be produced by whole cell extraction. For instance, the cells are first disrupted, and then intracellular and cell membrane/cell wall-associated lipids as well as extracellular hydrocarbons can be separated from the cell mass, such as by use of centrifugation. Intracellular lipids produced in the bacterium are, in some embodiments, extracted after lysing the cells of the bacterium. Additional methods of extracting bacterial lipids may be found in U.S. Pat. No. 8,592,188, which is incorporated herein by reference in its entirety.

The bacterial component can be produced from a bacterial source by a variety of methods. For instance, bacterial EVs can be separated from the bacterium by either destructive (e.g., grinding or blending of a bacterium) or non-destructive (washing or vacuum infiltration of a bacterium) methods. For instance, the bacterium can be vacuum-infiltrated, ground, blended, or a combination thereof to isolate EVs from the bacterium. For instance, the isolating step may involve vacuum infiltrating the bacterium (e.g., with a vesicle isolation buffer). Alternatively, the isolating step may involve grinding or blending the bacterium to release the EVs.

Upon isolating the bacterial EVs, the bacterial component can be separated or collected into a crude bacterial fraction (e.g., an apoplastic fraction). For instance, the separating step may involve separating the bacterial component into a crude bacterial fraction using centrifugation (e.g., differential centrifugation or ultracentrifugation) and/or filtration to separate the bacteria-containing fraction from large contaminants, including bacterial tissue debris or cells. As such, the crude bacterial fraction will have a decreased number of large contaminants, as compared to the initial sample from the bacterium. Depending on the method used, the crude bacterial fraction may additionally comprise a decreased level of bacterial cell organelles, as compared to the initial sample from the bacterium.

In some instances, the isolating step may involve centrifugation (e.g., differential centrifugation or ultracentrifugation) and/or filtration.

The crude bacterial fraction can be further purified by additional purification methods. For example, the crude bacterial fraction can be purified by ultracentrifugation, e.g., using a density gradient (iodixanol or sucrose) and/or use of other approaches to remove aggregated components (e.g., precipitation or size-exclusion chromatography). The resulting pure bacterial component may have a decreased level of contaminants or other undesired components from the bacterial source (e.g., protein aggregates, nucleic acid aggregates, protein-nucleic acid aggregates, free lipoproteins, lipido-proteic structures, nuclei, cell wall components, cell organelles, or a combination thereof) relative to one or more fractions generated during the earlier separation steps, or relative to a pre-established threshold level, e.g., a commercial release specification. For example, the pure bacterial component may have a decreased level (e.g., by about 5%, 10%, 15%, 20%, 30%, 40%, 50%, 60%, 70%, 80%, 90%, 100%, or more than 100%), or by about 2× fold, 4× fold, 5× fold, 10× fold, 20× fold, 25× fold, 50× fold, 75× fold, 100× fold, or more than 100× fold), or is substantially free of contaminants or other undesired components (e.g., protein aggregates, nucleic acid aggregates, protein-nucleic acid aggregates, free lipoproteins, lipido-proteic structures, nuclei, cell wall components, cell organelles, or a combination thereof) relative to the level in the initial sample.

For example, protein aggregates may be removed from the bacterial component. For example, the bacterial component can be taken through a range of pHs (e.g., as measured using a pH probe) to precipitate out protein aggregates in solution. The pH can be adjusted to, e.g., pH 3, pH 5, pH 7, pH 9, or pH 11 with the addition of, e.g., sodium hydroxide or hydrochloric acid. Once the solution is at the specified pH, it can be filtered to remove particulates. Alternatively, the bacterial component can be flocculated using the addition of charged polymers, such as Polymin-P or Praestol 2640. Briefly, Polymin-P or Praestol 2640 is added to the solution and mixed with an impeller. The solution can then be filtered to remove particulates. Alternatively, aggregates can be solubilized by increasing salt concentration. For example, NaCl can be added to the bacterial component until it is at, e.g., 1 mol/L. The solution can then be filtered to isolate the bacterial component. Alternatively, aggregates are solubilized by increasing the temperature. For example, the bacterial component can be heated under mixing until the solution has reached a uniform temperature of, e.g., 50° C. for 5 minutes. The bacterial component mixture can then be filtered. Alternatively, soluble contaminants from the bacterial component solutions can be separated by size-exclusion chromatography column according to standard procedures, where the bacterial lipids elute in the first fractions, whereas proteins and ribonucleoproteins and some lipoproteins are eluted later. The efficiency of protein aggregate removal can be determined by measuring and comparing the protein concentration before and after removal of protein aggregates via BCA/Bradford protein quantification.

Alternatively, the bacterial lipids extracted from a bacterial source may be obtained from a commercially available source.

Any of the production methods described herein can be supplemented with any quantitative or qualitative methods known in the art to characterize or identify the bacterial component (e.g., the bacterial lipids extracted from a bacterial source) at any step of the production process. For instance, the bacterial component (e.g., the bacterial lipids extracted from a bacterial source) may be characterized by a variety of analysis methods to estimate yield, concentration, purity, composition, or sizes, by a number of methods known in the art that enable visualization, quantitation, or qualitative characterization (e.g., identification of the composition), such as microscopy (e.g., transmission electron microscopy), dynamic light scattering, nanoparticle tracking, spectroscopy (e.g., Fourier transform infrared analysis), or mass spectrometry (protein and lipid analysis).

During the production process, the bacterial component (e.g., the bacterial lipids extracted from a bacterial source) can optionally be prepared such that the bacterial component is at an increased concentration (e.g., by about 5%, 10%, 15%, 20%, 30%, 40%, 50%, 60%, 70%, 80%, 90%, 100%, or more than 100%; or by about 2× fold, 4× fold, 5× fold, 10× fold, 20× fold, 25× fold, 50× fold, 75× fold, 100× fold, or more than 100× fold) relative to the level in a control or initial sample. The bacterial component may make up about 0.1% to about 100% of the bacteria-derived lipid composition, for instance, at about 0.01% to about 100%, about 1% to about 99.9%, about 0.1% to about 10%, about 1% to about 25%, about 10% to about 50%, about 50% to about 99%, or about 75% to about 100%.

### The Bacteria-Derived Lipid Composition—Lipid Modification of the Bacterial Component

The bacteria-derived lipid composition comprises (a) a bacterial component; and (b) an ionizable lipid. The ionizable lipid and/or other exogenous lipids are used to modify the bacterial component.

The modification refers to modifying a bacterial component containing a lipid structure (e.g., a lipid bilayer, unilamellar, multilamellar structure; e.g., a vesicular lipid structure) derived from (e.g., enriched, isolated or purified from) a bacterial source, wherein the lipid structure is disrupted (e.g., disrupted by lipid extraction) and reassembled or reconstituted in a liquid phase (e.g., a liquid phase containing a cargo) using standard methods, e.g., reconstituted by a method comprising lipid film hydration and/or solvent injection, to produce the bacteria-derived lipid composition, as is described herein. In some embodiments, the bacterial component is modified by reconstructing a film comprising the bacterial component in the presence of the ionizable lipid.

In some embodiments, the bacterial component is modified by reconstructing a film comprising the purified bacteria lipids of the bacterial component with the ionizable lipid.

Alternatively, the bacteria-derived lipid composition may be produced using a microfluidic device (such as a NanoAssemblr® IGNITE™ microfluidic instrument (Precision NanoSystems)).

In some embodiments, the bacteria-derived lipid composition is produced by a process comprising the steps of (a) providing a bacterial component (e.g., the bacterial component purified as described above); (b) processing the bacterial component to produce a lipid film; (c) reconstituting the lipid film in an organic solvent or solvent combination, thereby producing a lipid solution; and (d) processing the lipid solution of step (c) in a microfluidics device comprising an aqueous phase, in the presence of the ionizable lipid, thereby producing the bacteria-derived lipid composition.

In some instances, processing the bacterial component to produce a lipid film includes extracting lipids using the Bligh-Dyer method (Bligh and Dyer, J Biolchem Physiol, 37: 911-917, 1959), which is incorporated herein by reference in its entirety. The extracted lipids may be provided as a stock solution, e.g., a solution in chloroform:methanol. Producing the lipid film may comprise, e.g., evaporation of the solvent with a stream of inert gas (e.g., nitrogen).

The method may, if desired, further comprise sonication, freeze/thaw treatment, and/or lipid extrusion, e.g., to reduce the size of the reconstituted bacteria-derived lipid composition.

### Bacterial Lipids

A bacteria-derived lipid composition may comprise between 10% and 100% lipids derived from the lipid structure from the bacterial source, e.g., it may contain at least 10%, at least 20%, at least 30%, at least 40%, at least 50%, at least 60%, at least 70%, at least 80%, at least 90%, at least 95%, or 100% lipids derived from the lipid structure from the bacterial source (e.g., E. coli). A bacteria-derived lipid composition may comprise all or a fraction of the lipid species present in the lipid structure from the bacteria-derived lipid composition (e.g., E. coli), e.g., it may contain at least 10%, at least 20%, at least 30%, at least 40%, at least 50%, at least 60%, at least 70%, at least 80%, at least 90%, or 100% of the lipid species present in the lipid structure from the bacterial source. A bacteria-derived lipid composition may comprise none, a fraction, or all of the protein species present in the lipid structure from the bacterial source, e.g., it may contain 0%, less than 1%, less than 5%, less than 10%, less than 15%, less than 20%, less than 30%, less than 40%, less than 50%, less than 60%, less than 70%, less than 80%, less than 90%, less than 100%, or 100% of the protein species present in the lipid structure from the bacterial source. In some instances, the lipid bilayer of the bacteria-derived lipid composition does not contain proteins. In some instances, the lipid structure of the bacteria-derived lipid composition contains a reduced amount of proteins relative to the lipid structure from the bacterial source.

In some embodiments, the bacterial lipids of the bacteria-derived lipid composition are extracted from Escherichia (e.g., E. coli) or Salmonella (e.g., Salmonella typhimurium).

### Exogenous Lipids

The bacterial component may be modified to contain a heterologous agent (e.g., a cell-penetrating agent) that is capable of increasing cell uptake (e.g., animal cell uptake (e.g., mammalian cell uptake, e.g., human cell uptake), plant cell uptake, bacterial cell uptake, or fungal cell uptake) relative to an unmodified bacterial component. For example, the modified bacterial component may include (e.g., be loaded with, e.g., encapsulate or be conjugated to) or be formulated with (e.g., be suspended or resuspended in a solution comprising) a cell-penetrating agent, such as an ionizable lipid. Each of the modified bacterial component may comprise at least 1%, 5%, 10%, 20%, 30%, 40%, 50%, 60%, 70%, 80%, 90%, or more than 90% ionizable lipid.

The bacteria-derived lipid composition may include one or more exogenous lipids, e.g., lipids that are exogenous to the bacterium (e.g., originating from a source that is not the bacterium from which the bacterial component is produced). The lipid composition of the bacteria-derived lipid composition may include 0%, less than 1%, or at least 1%, 2%, 5%, 10%, 15%, 20%, 25%, 30%, 40%, 50%, 60%, 70%, 80%, 90%, 95%, or more than 95% exogenous lipid. In some examples, the exogenous lipid (e.g., ionizable lipid) is added to amount to 25% or 40% (w/w) of total lipids in the preparation. In some examples, the exogenous lipid is added to the preparation prior to step (b), e.g., mixed with extracted bacterial lipids prior to step (b).

Exemplary exogenous lipids include ionizable lipids.

Exogenous lipids may also include cationic lipids.

In some instances, the exogenous lipid may be an ionizable lipid or cationic lipid chosen from 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), DLin-MC3-DMA (MC3), dioleoyl-3-trimethylammonium propane (DODAP), DC-cholesterol, DOTAP, Ethyl PC, GL67, DLin-KC2-DMA (KC2), MD1 (cKK-E12), OF2, EPC, ZA3-Ep10, TT3, LP01, 5A2-SC8, Lipid 5 (Moderna), a cationic sulfonamide amino lipid, an amphiphilic zwitterionic amino lipid, DODAC, DOBAQ, YSK05, DOBAT, DOBAQ, DOPAT, DOMPAQ, DOAAQ, DMAP-BLP, DLinDMA, DODMA, DOTMA, DSDMA, DOSPA, DODAC, DOBAQ, DMRIE, DOTAP-cholesterol, GL67A, and 98N12-5, and a combination thereof.

In some embodiments, the exogenous lipid may be an ionizable lipid or cationic lipid chosen from C12-200, MC3, DODAP, DC-cholesterol, DOTAP, Ethyl PC, GL67, KC2, MD1, OF2, EPC, ZA3-Ep10, TT3, LP01, 5A2-SC8, Lipid 5 (Moderna), a cationic sulfonamide amino lipid, and an amphiphilic zwitterionic amino lipid and a combination thereof. In some embodiments, the ionizable lipid is chosen from C12-200, MC3, DODAP, and DC-cholesterol or combinations thereof. In some instances, the ionizable lipid is an ionizable lipid. In some embodiments, the ionizable lipid is 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200) or (6Z,9Z,28Z,31Z)-Heptatriaconta-6,9,28,31-tetraen-19-yl 4-(dimethylamino)butanoate, DLin-MC3-DMA (MC3). In some instances, the exogenous lipid is a cationic lipid. In some embodiments, the cationic lipid is DC-cholesterol or dioleoyl-3-trimethylammonium propane (DOTAP).

In some instances, the bacteria-derived lipid composition comprises at least 1%, 5%, 10%, 20%, 30%, 40%, 50%, 60%, 70%, 80%, 90%, or more than 90% ionizable lipid.

In some instances, the bacteria-derived lipid composition comprises least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 30%-75% ionizable lipid (e.g., about 30%-75% ionizable lipid), in mol %. In some embodiments, the bacteria-derived lipid composition comprises 25% C12-200, in mol %. In some embodiments, the bacteria-derived lipid composition comprises 35% C12-200, in mol %. In some embodiments, the bacteria-derived lipid composition comprises 50% C12-200, in mol %. In some embodiments, the bacteria-derived lipid composition comprises 40% MC3, in mol %. In some embodiments, the bacteria-derived lipid composition comprises 50% C12-200, in mol %. In some embodiments, the bacteria-derived lipid composition comprises 20% or 40% DC-cholesterol, in mol %. In some embodiments, the bacteria-derived lipid compositions comprises 25% or 40% DOTAP, in mol %.

The agent may increase uptake of the bacteria-derived lipid composition as a whole or may increase uptake of a portion or component of the bacteria-derived lipid composition (e.g., the heterologous functional agent) carried by the bacteria-derived lipid composition. The degree to which cell uptake is increased may vary depending on the bacterium to which the composition is delivered, the bacteria-derived lipid composition, and other modifications made to the bacteria-derived lipid composition. For example, the bacteria-derived lipid composition may have an increased cell uptake (e.g., animal cell uptake, plant cell uptake, bacterial cell uptake, or fungal cell uptake) of at least 1%, 2%, 5%, 10%, 15%, 20%, 30%, 40%, 50%, 60%, 70%, 80%, 90%, or 100% relative to an unmodified bacterial component. In some instances, the increased cell uptake is an increased cell uptake of at least 2×-fold, 4×-fold, 5×-fold, 10×-fold, 100×-fold, or 1000×-fold relative to an unmodified bacterial component.

In some embodiments, a bacteria-derived lipid composition that has been modified with a ionizable lipid more efficiently encapsulates a negatively charged a polynucleotide than a bacteria-derived lipid composition that has not been modified with an ionizable lipid. In some aspects, a bacteria-derived lipid composition that has been modified with an ionizable lipid has altered biodistribution relative to a bacteria-derived lipid composition that has not been modified with an ionizable lipid. In some aspects, a bacteria-derived lipid composition that has been modified with an ionizable lipid has altered (e.g., increased) fusion with an endosomal membrane of a target cell relative to a bacteria-derived lipid composition that has not been modified with an ionizable lipid.

### Ionizable Lipids

In some embodiments, the ionizable lipid has at least one (e.g., one, two, three, four or all five) of the characteristics listed below:
(i) at least 2 ionizable amines (e.g., at least 2, at least 3, at least 4, at least 5, at least 6, or more than 6 ionizable amines, e.g., 2, 3, 4, 5, 6, 7, 8, 9, 10, 11, 12, or more than 12 ionizable amines);
(ii) at least 3 lipid tails (e.g., at least 3, at least 4, at least 5, at least 6, or more than 6 lipid tails, e.g., 2, 3, 4, 5, 6, 7, 8, 9, 10, 11, 12, or more than 12 lipid tails), wherein each of the lipid tails is independently at least 6 carbon atoms in length (e.g., at least 6, at least 7, at least 8, at least 9, at least 10, at least 11, at least 12, at least 13, at least 14, at least 15, at least 16, at least 17, at least 18, or more than 18 carbon atoms in length, e.g., 6, 7, 8, 9, 10, 11, 12, 13, 14, 15, 16, 17, 18, 19, 20, 21, 22, 23, 24, 25, or more than 25 carbon atoms in length);
(iii) an acid dissociation constant (pKa) of from about 4.5 to about 7.5 (e.g., a pKa of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5 (e.g., a pKa of from about 6.5 and about 7.5 (e.g., a pKa of about 6.5, 6.6, 6.7, 6.8, 6.9, 7.0, 7.1, 7.2, 7.3, 7.4, or 7.5));
(iv) an ionizable amine and a heteroorganic group; and
(v) an N:P (amines of ionizable lipid:phosphates of mRNA) ratio of at least 10.

In some embodiments, the BacLC has an N/P ratio of about 12 to about 17, for example the N/P ratio is about 15±1, or the N/P ratio is about 15±0.5. In some embodiments, the N/P ratio is about 15. Alternatively, the ionizable lipid is characterized by an N/P ratio of about 3 to about 10, for example the N/P ratio is about 6±1, or the N/P ratio is about 6±0.5. In some embodiments, the N/P ratio is about 6.

In some embodiments, the ionizable lipid is not selected from 1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5 (Moderna), and 98N12-5.

In some embodiments, the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

In some embodiments, the ionizable lipid is an ionizable amine and a heteroorganic group. In some embodiments, the heteroorganic group is hydroxyl. In some embodiments, the heteroorganic group comprises a hydrogen bond donor. In some embodiments, the heteroorganic group comprises a hydrogen bond acceptor. In some embodiments, the heteroorganic group is —OH, —SH, —(CO)H, —CO₂H, —NH₂, —CONH₂, optionally substituted C₁-C₆ alkoxy, or fluorine.

In some embodiments, the ionizable lipid is an ionizable amine and a heteroorganic group separated by a chain of at least two atoms

In some embodiments, the ionizable lipid is represented by the following formula I:

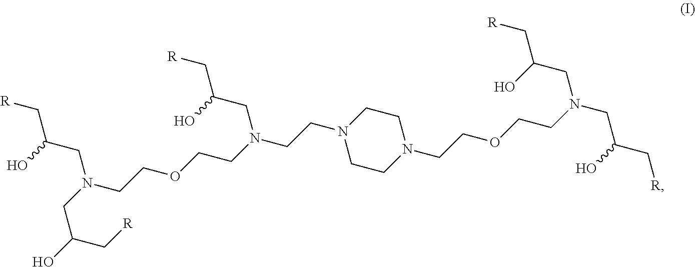

wherein R is C₈-C₁₄ alkyl group.

In some embodiments, a lipid membrane of the bacteria-derived lipid composition comprises at least 35% of the lipid of formula I, e.g., at least 40%, 45%, 50%, 55%, 60%, 65%, 70%, 75% 80%, 85%, 90%, or more than 90% of the lipid of formula I, e.g., 35%-40%, 40%-50%, 50%-60%, 60%-70%, 70%-80%, or 80%-90% of the lipid of formula I.

In some instances, the bacteria-derived lipid composition comprises at least 1%, 5%, 10%, 20%, 30%, 40%, 50%, 60%, 70%, 80%, 90%, or more than 90% ionizable lipid.

In some instances, the bacteria-derived lipid composition comprises at least 0.1%, 1%, 5%, 10%, 15%, 20%, 25%, 30%, 35%, 40%, 45%, 50%, 55%, 60%, 65%, 70%, 75% 80%, 85%, 90%, or more than 90% ionizable lipid, e.g., 1%-10%, 10%-20%, 20%-30%, 30%-40%, 40%-50%, 50%-60%, 60%-70%, 70%-80%, or 80%-90% ionizable lipid, e.g., about 25%-75% ionizable lipid (e.g., about 25%-75% ionizable lipid), all in mol %.

The ionizable lipid described herein may include an amine core described herein substituted with one or more (e.g., 1, 2, 3, 4, 5, or 6) lipid tails. In some embodiments, the ionizable lipid described herein include at least 3 lipid tails. A lipid tail may be a C₈-C₁₈ hydrocarbon (e.g., C₆-C₁₈ alkyl or C₆-C₁₈ alkanoyl). An amine core may be substituted with one or more lipid tails at a nitrogen atom (e.g., one hydrogen atom attached to the nitrogen atom may be replaced with a lipid tail).

In some embodiments, the amine core has a structure of:

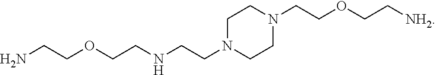 [SMILES: NCCOCCNCCN1CCN(CCOCCN)CC1]

In some embodiments, the amine core has a structure of:

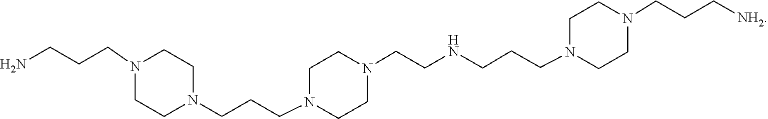 [SMILES: NCCCN1CCN(CCCNCCN2CCN(CCCN3CCN(CCCN)CC3)CC2)CC1]

In some embodiments, the amine core has a structure of:

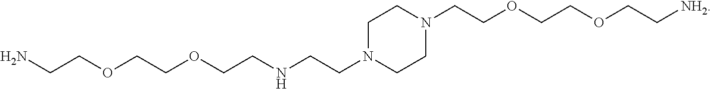 [SMILES: NCCOCCOCCNCCN1CCN(CCOCCOCCN)CC1]

In some embodiments, the amine core has a structure of:

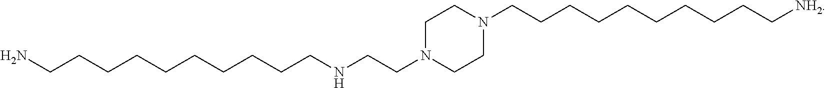 [SMILES: NCCCCCCCCCCNCCN1CCN(CCCCCCCCCCN)CC1]

In some embodiments, the amine core has a structure of:

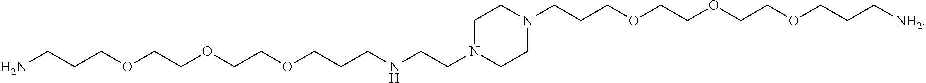 [SMILES: NCCCOCCOCCOCCCNCCN1CCN(CCCOCCOCCOCCCN)CC1]

In some embodiments, the amine core has a structure of:

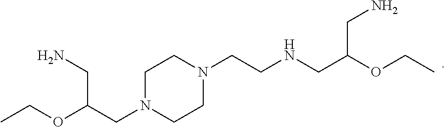 [SMILES: CCOC(CN)CNCCN1CCN(CC(CN)OCC)CC1]

In some embodiments, the amine core has a structure of:

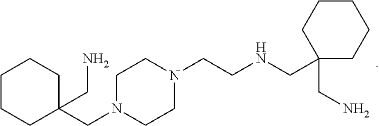 [SMILES: NCC1(CNCCN2CCN(CC3(CN)CCCCC3)CC2)CCCCC1]

In some embodiments, the amine core has a structure of:

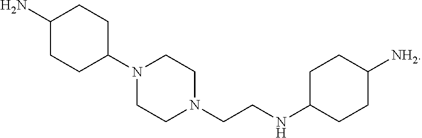 [SMILES: NC1CCC(NCCN2CCN(C3CCC(N)CC3)CC2)CC1]

The bacteria-derived lipid composition may further contain cationic lipids.

Other suitable lipids for use in the bacteria-derived lipid composition and methods for making and using thereof include the lipids as described in International Patent Publication WO 2016/118725, which is incorporated herein by reference in its entirety.

In certain embodiments, the bacteria-derived lipid composition and methods for making and using thereof include a lipid having a compound structure of:

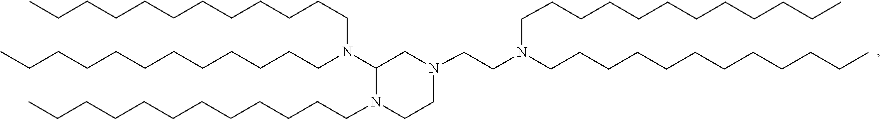 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable lipids for use in the bacteria-derived lipid composition and methods for making and using thereof include the lipids as described in International Patent Publication WO 2016/118724, which is incorporated herein by reference in its entirety.

In certain embodiments, the bacteria-derived lipid composition and methods for making and using thereof include a lipid having a compound structure of:

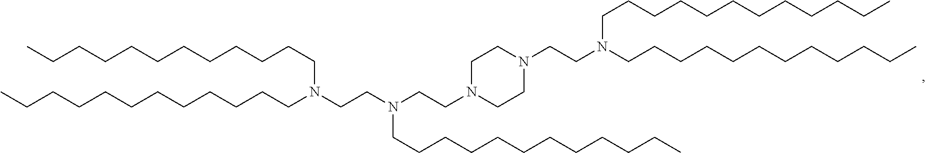 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable lipids for use in the bacteria-derived lipid composition and methods for making and using thereof include a lipid having the formula of 14,25-ditridecyl 15,18,21,24-tetraaza-octatriacontane, and pharmaceutically acceptable salts thereof.

Other suitable lipids for use in the bacteria-derived lipid composition and methods for making and using thereof include the lipids as described in International Patent Publications WO 2013/063468 and WO 2016/205691, each of which is incorporated herein by reference in its entirety.

In some embodiments, the bacteria-derived lipid composition and methods for making and using thereof include a lipid of the following formula:

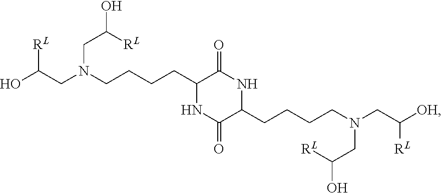

or pharmaceutically acceptable salts thereof, wherein each instance of R^{L} is independently optionally substituted C₆-C₄₀ alkenyl.

In certain embodiments, the bacteria-derived lipid composition and methods for making and using thereof include a lipid having a compound structure of:

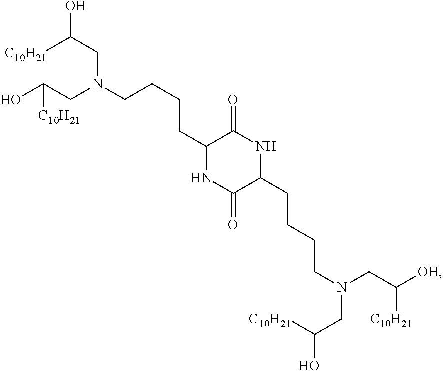

and pharmaceutically acceptable salts thereof.

In certain embodiments, the bacteria-derived lipid composition and methods for making and using thereof include a lipid having a compound structure of:

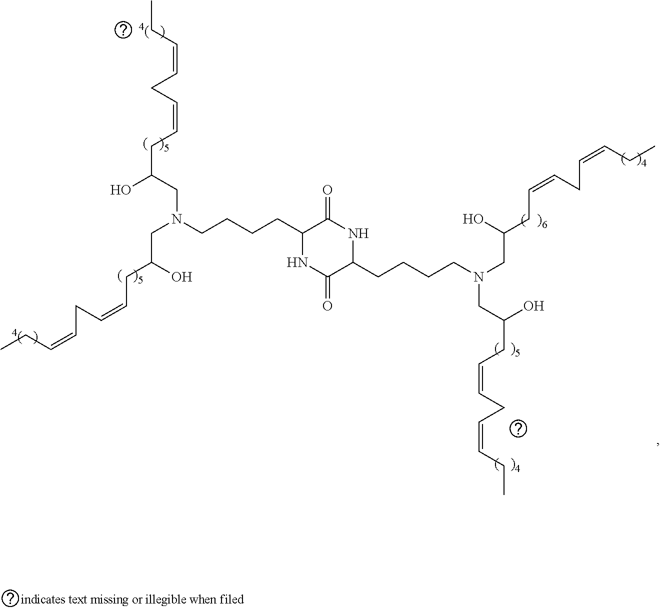

and pharmaceutically acceptable salts thereof.

In certain embodiments, the bacteria-derived lipid composition and methods for making and using thereof include a lipid having a compound structure of:

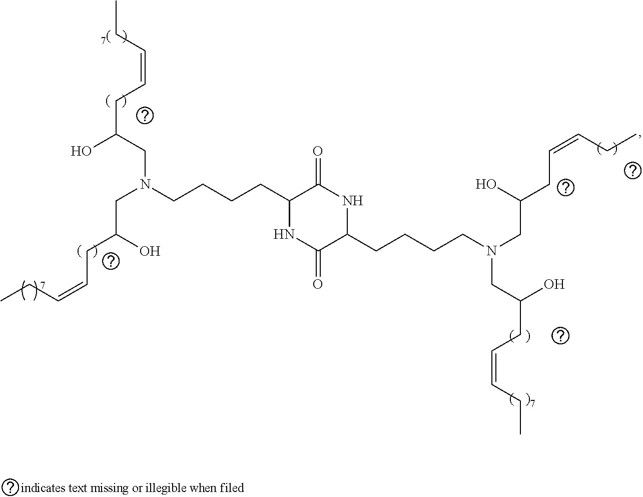

and pharmaceutically acceptable salts thereof.

In certain embodiments, the bacteria-derived lipid composition and methods for making and using thereof include a lipid having a compound structure of:

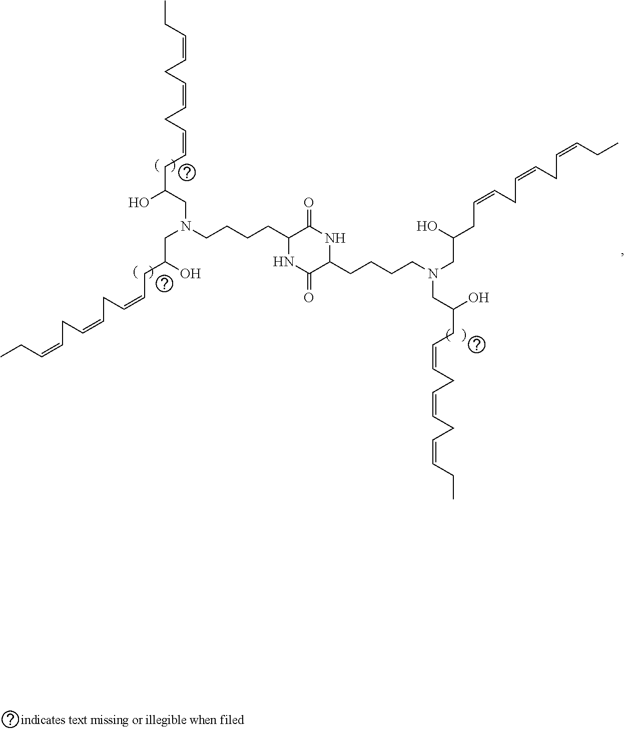

and pharmaceutically acceptable salts thereof.

Other suitable lipids for use in the bacteria-derived lipid composition and methods for making and using thereof include the lipids as described in International Patent Publication WO 2015/184256, which is incorporated herein by reference in its entirety. In some embodiments, the bacteria-derived lipid composition and methods for making and using thereof include a lipid of the following formula:

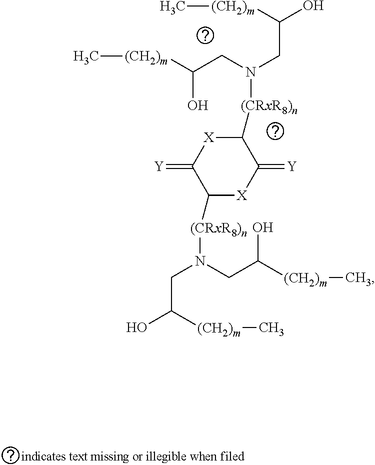

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen.

In certain embodiments, the bacteria-derived lipid composition and methods for making and using thereof include a lipid, “Target 23”, having a compound structure of:

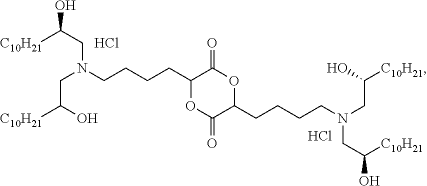

(Target 23) and pharmaceutically acceptable salts thereof.

Other suitable lipids for use in the bacteria-derived lipid composition and methods for making and using thereof include the lipids as described in International Patent Publication WO 2016/004202, which is incorporated herein by reference in its entirety.

In some embodiments, the bacteria-derived lipid composition and methods for making and using thereof include a lipid having the compound structure:

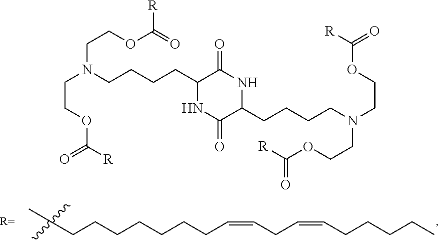

or a pharmaceutically acceptable salt thereof.

In some embodiments, the bacteria-derived lipid composition and methods for making and using thereof include a lipid having the compound structure:

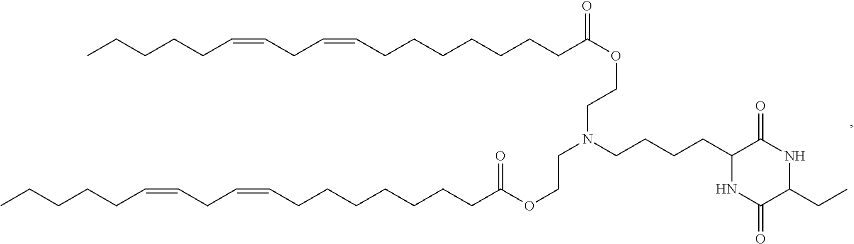 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof.

In some embodiments, the bacteria-derived lipid composition and methods for making and using thereof include a lipid having the compound structure:

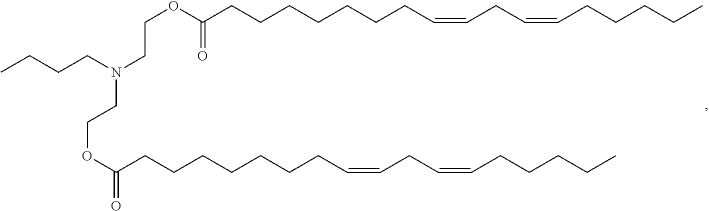 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable lipids for use in the bacteria-derived lipid composition and methods for making and using thereof include lipids as described in U.S. Provisional Patent Application Ser. No. 62/758,179, which is incorporated herein by reference in its entirety.

In some embodiments, the bacteria-derived lipid composition and methods for making and using thereof include a lipid of the following formula:

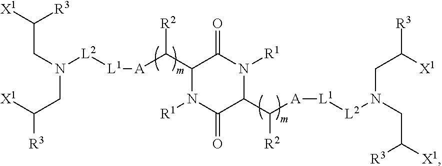

or a pharmaceutically acceptable salt thereof, wherein each R¹ and R² is independently H or C1-C6 aliphatic; each m is independently an integer having a value of 1 to 4; each A is independently a covalent bond or arylene; each L¹ is independently an ester, thioester, disulfide, or anhydride group; each L² is independently C2-C10 aliphatic; each X¹ is independently H or OH; and each R³ is independently C6-C20 aliphatic.

In some embodiments, the bacteria-derived lipid composition and methods for making and using thereof include a lipid of the following formula:

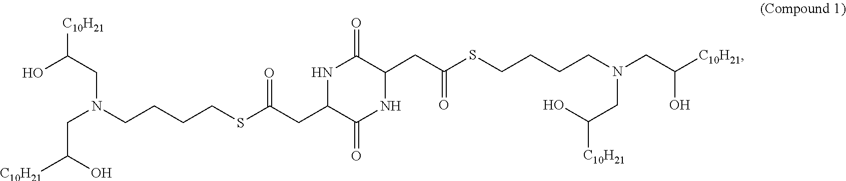

or a pharmaceutically acceptable salt thereof.

In some embodiments, the bacteria-derived lipid composition and methods for making and using thereof include a lipid of the following formula:

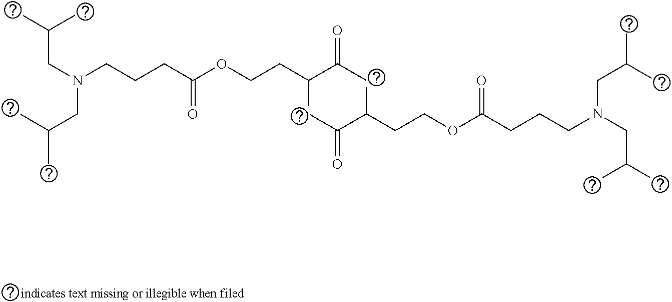 [SMILES: CC(C)CN(CCCC(=O)OCCC1CC(=O)C(CCOC(=O)CCCN(CC(C)C)CC(C)C)CC1=O)CC(C)C]

or a pharmaceutically acceptable salt thereof.

In some embodiments, the bacteria-derived lipid composition and methods for making and using thereof include a lipid of the following formula:

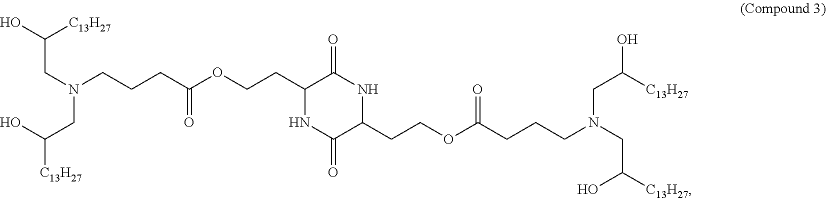

or a pharmaceutically acceptable salt thereof.

Other suitable lipids for use in the bacteria-derived lipid composition and methods for making and using thereof include the lipids as described in J. McClellan, M. C. King, Cell 2010, 141, 210-217 and in Whitehead et al., Nature Communications (2014) 5:4277, which is incorporated herein by reference in its entirety.

In certain embodiments, the lipids of the bacteria-derived lipid composition and methods for making and using thereof include a lipid having a compound structure of:

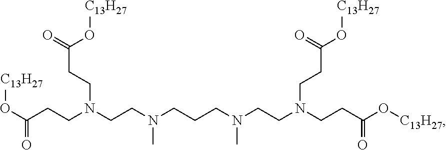

and pharmaceutically acceptable salts thereof.

Other suitable lipids for use in the bacteria-derived lipid composition and methods for making and using thereof include the lipids as described in International Patent Publication WO 2015/199952, which is incorporated herein by reference in its entirety.

In some embodiments, the bacteria-derived lipid composition and methods for making and using thereof include a lipid having the compound structure:

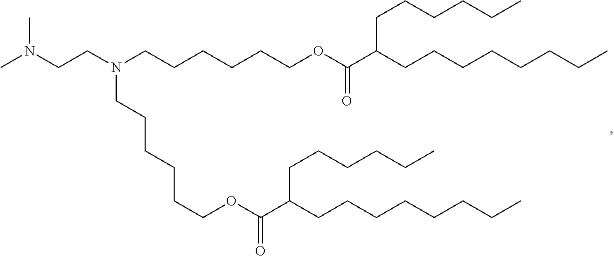 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof.

In some embodiments, the bacteria-derived lipid composition and methods for making and using thereof include a lipid having the compound structure:

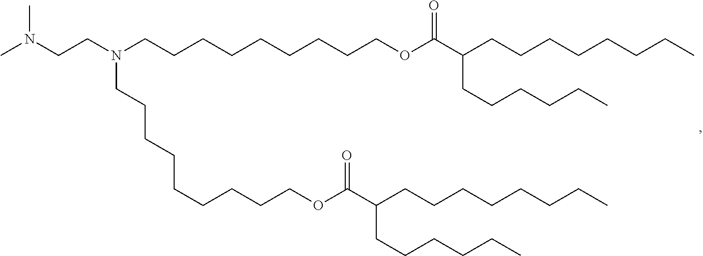 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof.

In some embodiments, the bacteria-derived lipid composition and methods for making and using thereof include a lipid having the compound structure:

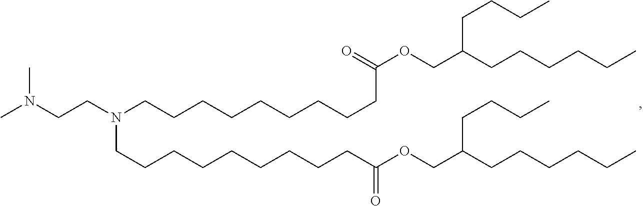 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof.

In some embodiments, the bacteria-derived lipid composition and methods for making and using thereof include a lipid having the compound structure:

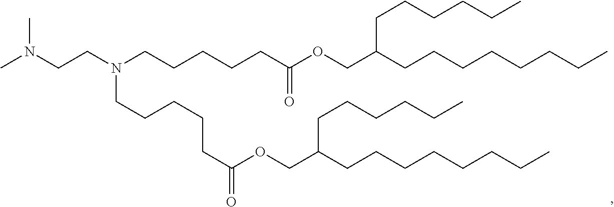 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof.

In some embodiments, the bacteria-derived lipid composition and methods for making and using thereof include a lipid having the compound structure:

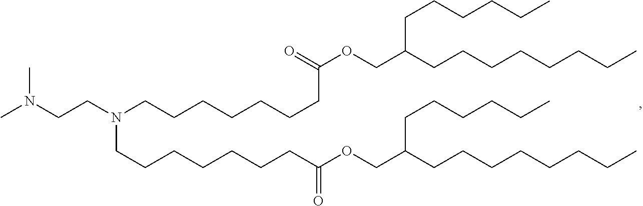 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof.

In some embodiments, the bacteria-derived lipid composition and methods for making and using thereof include a lipid having the compound structure:

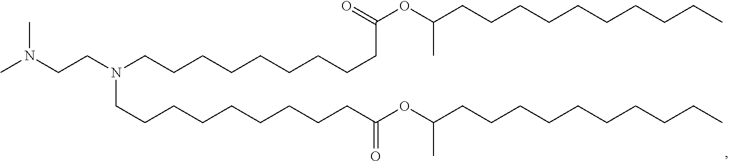 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof.

In some embodiments, the bacteria-derived lipid composition and methods for making and using thereof include a lipid having the compound structure:

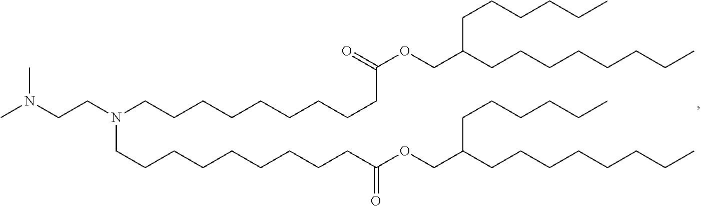 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof.

In some embodiments, the bacteria-derived lipid composition and methods for making and using thereof include a lipid having the compound structure:

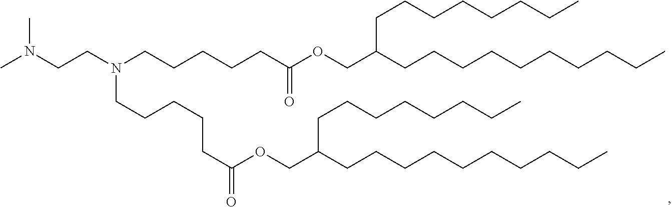 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof.

In some embodiments, the bacteria-derived lipid composition and methods for making and using thereof include a lipid having the compound structure:

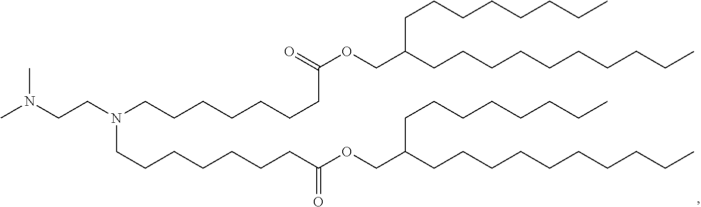 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof.

In some embodiments, the bacteria-derived lipid composition and methods for making and using thereof include a lipid having the compound structure:

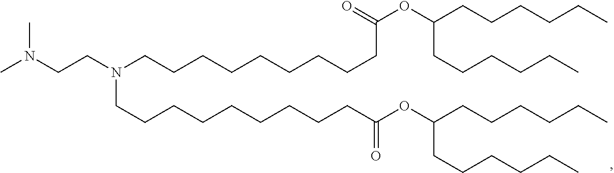 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof.

In some embodiments, the bacteria-derived lipid composition and methods for making and using thereof include a lipid having the compound structure:

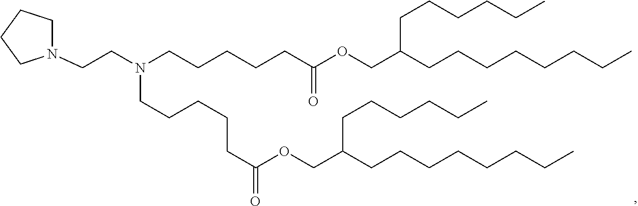 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof.

In some embodiments, the bacteria-derived lipid composition and methods for making and using thereof include a lipid having the compound structure:

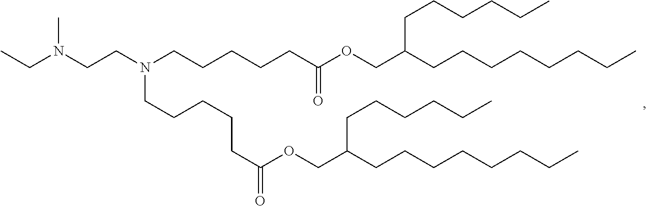 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof.

In some embodiments, the bacteria-derived lipid composition and methods for making and using thereof include a lipid having the compound structure:

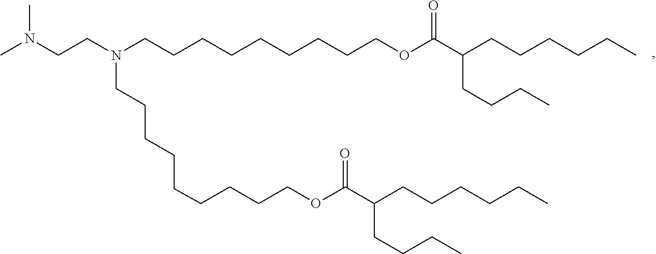 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable lipids for use in the bacteria-derived lipid composition and methods for making and using thereof include the lipids as described in International Patent Publication WO 2017/004143, which is incorporated herein by reference in its entirety.

In some embodiments, the bacteria-derived lipid composition and methods for making and using thereof include a lipid having the compound structure;

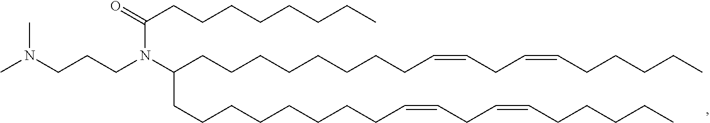 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof.

In some embodiments, the bacteria-derived lipid composition and methods for making and using thereof include a lipid having the compound structure:

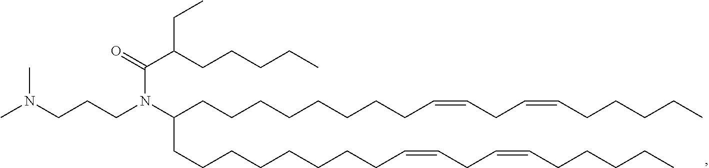 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof.

In some embodiments, the bacteria-derived lipid composition and methods for making and using thereof include a lipid having the compound structure:

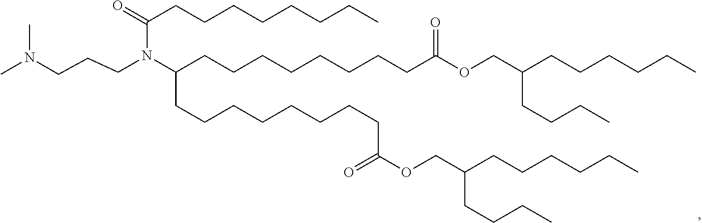 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof.

In some embodiments, the bacteria-derived lipid composition and methods for making and using thereof include a lipid having the compound structure:

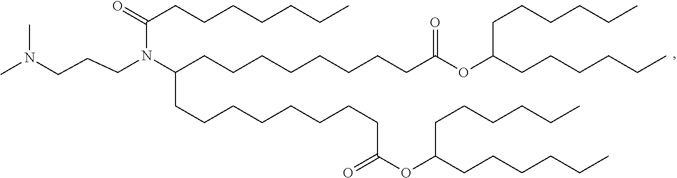 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the bacteria-derived lipid composition and methods for making and using thereof include a lipid having the compound structure:

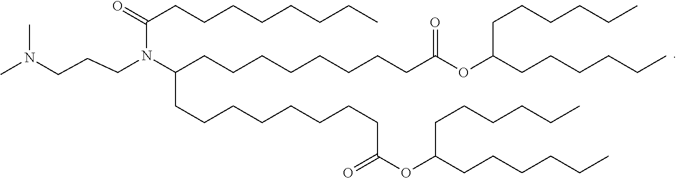 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof.

In some embodiments, the bacteria-derived lipid composition and methods for making and using thereof include a lipid having the compound structure:

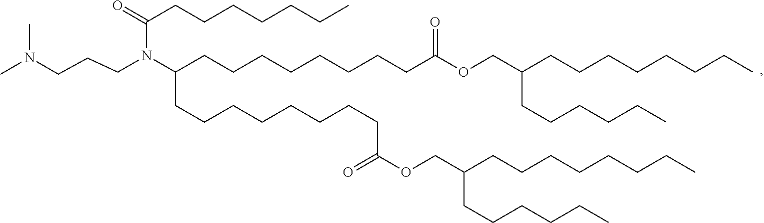 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof.

In some embodiments, the bacteria-derived lipid composition and methods for making and using thereof include a lipid having the compound structure:

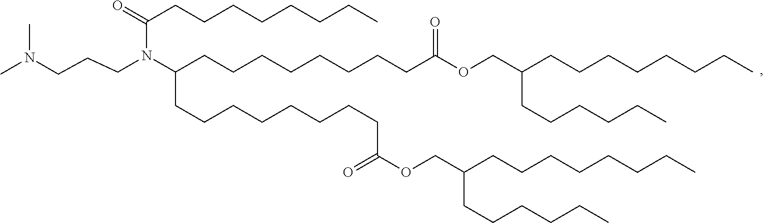 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof.

In some embodiments, the bacteria-derived lipid composition and methods for making and using thereof include a lipid having the compound structure:

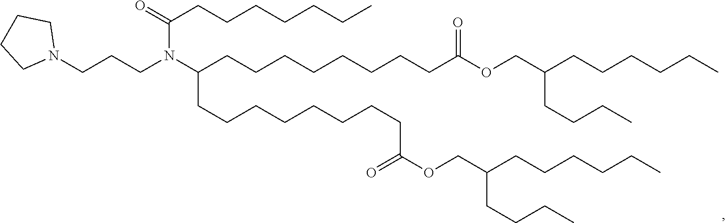 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the bacteria-derived lipid composition and methods for making and using thereof include a lipid having the compound structure:

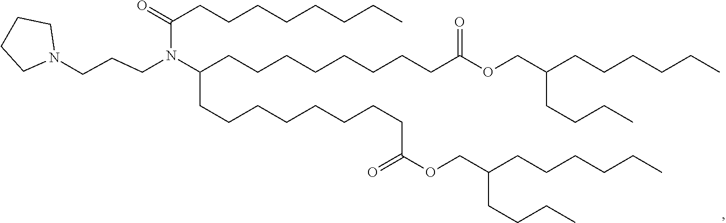 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof.

In some embodiments, the bacteria-derived lipid composition and methods for making and using thereof include a lipid having the compound structure:

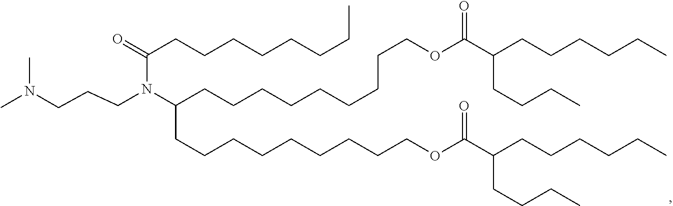 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof.

In some embodiments, the bacteria-derived lipid composition and methods for making and using thereof include a lipid having the compound structure:

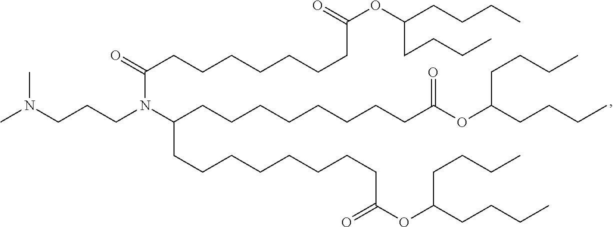 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof.

In some embodiments, the bacteria-derived lipid composition and methods for making and using thereof include a lipid having the compound structure:

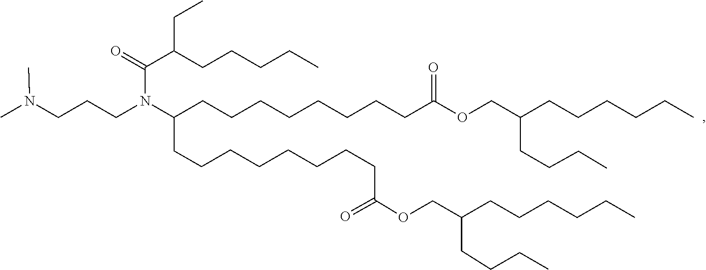 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof.

In some embodiments, the bacteria-derived lipid composition and methods for making and using thereof include a lipid having the compound structure:

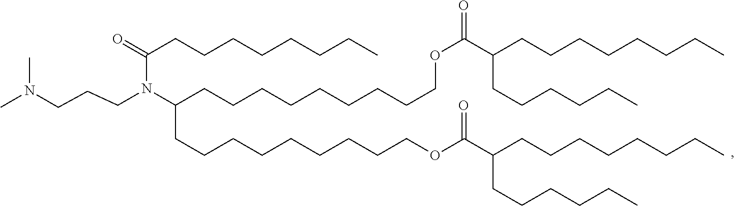 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof.

In some embodiments, the bacteria-derived lipid composition and methods for making and using thereof include a lipid having the compound structure:

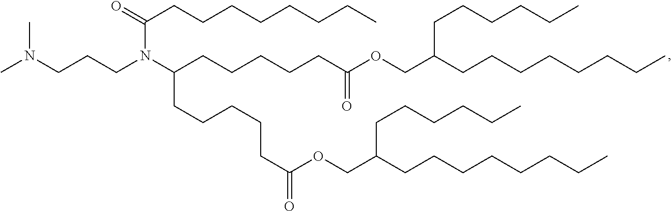 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof.

In some embodiments, the bacteria-derived lipid composition and methods for making and using thereof include a lipid having the compound structure:

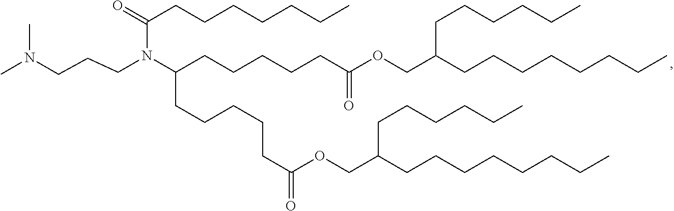 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof.

In some embodiments, the bacteria-derived lipid composition and methods for making and using thereof include a lipid having the compound structure:

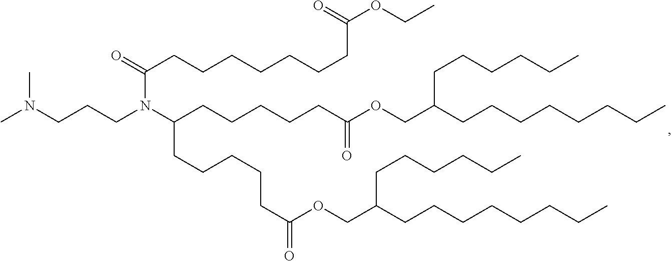 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof.

In some embodiments, the bacteria-derived lipid composition and methods for making and using thereof include a lipid having the compound structure:

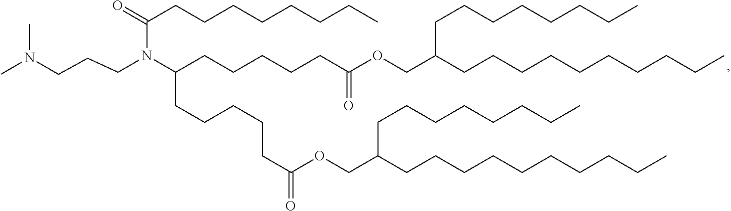 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable lipids for use in the bacteria-derived lipid composition and methods for making and using thereof include the lipids as described in International Patent Publication WO 2017/075531, which is incorporated herein by reference in its entirety.

In some embodiments, the bacteria-derived lipid composition and methods for making and using thereof include a lipid of the following formula:

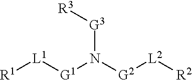

or a pharmaceutically acceptable salt thereof, wherein one of L¹ or L² is —O(C═O)—, —(C═O)O—, —C(═O)—, —O—, —S(O)x, —S—S—, —C(═O)S—, —SC(═O)—, —NRaC(═O)—, —C(═O)NR^{a}—, NRaC(═O)NR^{a}—, —OC(═O)NR^{a}—, or —NRaC(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable lipids for use in the bacteria-derived lipid composition and methods for making and using thereof include the lipids as described in International Patent Publication WO 2017/117528, which is incorporated herein by reference in its entirety. In some embodiments, the bacteria-derived lipid composition and methods for making and using thereof include a lipid having the compound structure:

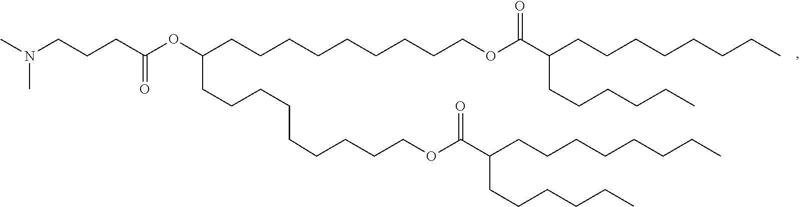 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof.

In some embodiments, the bacteria-derived lipid composition and methods for making and using thereof include a lipid having the compound structure:

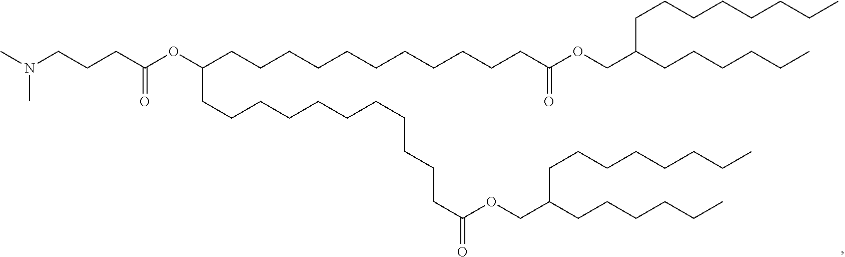 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof.

In some embodiments, the bacteria-derived lipid composition and methods for making and using thereof include a lipid having the compound structure:

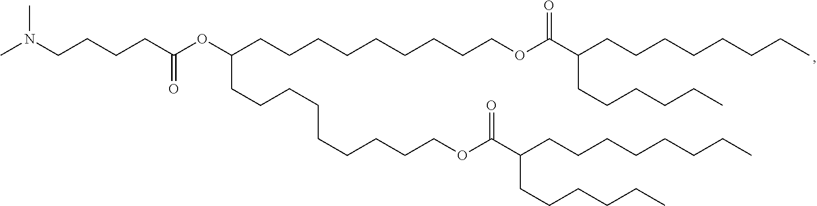 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable lipids for use in the bacteria-derived lipid composition and methods for making and using thereof include the lipids as described in International Patent Publication WO 2017/049245, which is incorporated herein by reference in its entirety.

In some embodiments, the lipids of the bacteria-derived lipid composition and methods for making and using thereof include a compound of one of the following formulas:

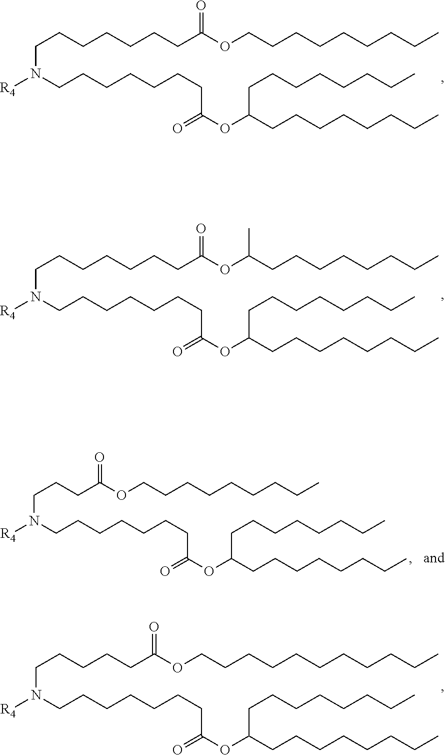

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3.

In certain embodiments, the bacteria-derived lipid composition and methods for making and using thereof include a lipid having a compound structure of:

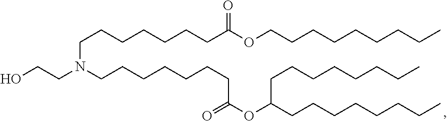 [SMILES: CCCCCCCCCOC(=O)CCCCCCCN(CCO)CCCCCCCC(=O)OC(CCCCCCCC)CCCCCCCC]

and pharmaceutically acceptable salts thereof.

In certain embodiments, the bacteria-derived lipid composition and methods for making and using thereof include a lipid having a compound structure of:

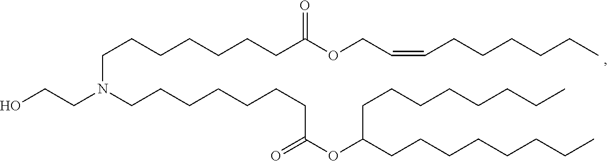 [SMILES: CCCCCC/C=C\COC(=O)CCCCCCCN(CCO)CCCCCCCC(=O)OC(CCCCCCCC)CCCCCCCC]

and pharmaceutically acceptable salts thereof.

In certain embodiments, the bacteria-derived lipid composition and methods for making and using thereof include a lipid having a compound structure of:

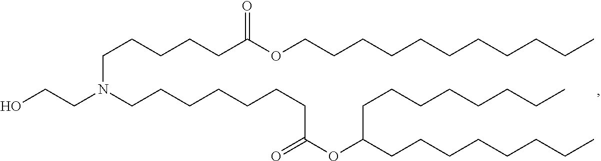 [SMILES: CCCCCCCCCCCOC(=O)CCCCCN(CCO)CCCCCCCC(=O)OC(CCCCCCCC)CCCCCCCC]

and pharmaceutically acceptable salts thereof.

In certain embodiments, the bacteria-derived lipid composition and methods for making and using thereof include a lipid having a compound structure of:

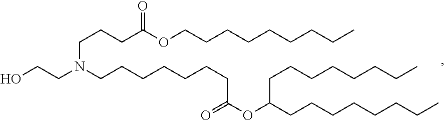 [SMILES: CCCCCCCCCOC(=O)CCCN(CCO)CCCCCCCC(=O)OC(CCCCCCCC)CCCCCCCC]

and pharmaceutically acceptable salts thereof.

Other suitable lipids for use in the bacteria-derived lipid composition and methods for making and using thereof include the lipids as described in International Patent Publication WO 2017/173054 and WO 2015/095340, each of which is incorporated herein by reference in its entirety. In certain embodiments, the bacteria-derived lipid composition and methods for making and using thereof include a lipid having a compound structure of:

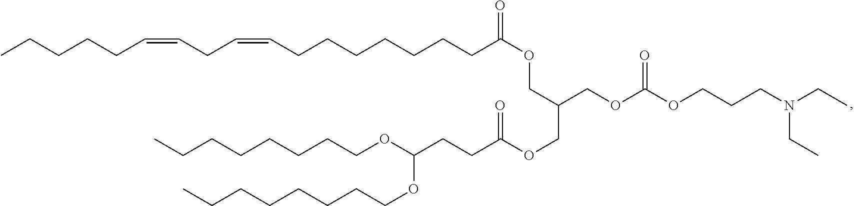 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof.

In certain embodiments, the bacteria-derived lipid composition and methods for making and using thereof include a lipid having a compound structure of:

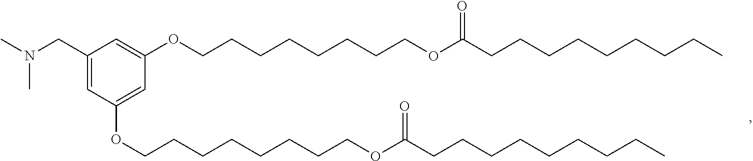 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof.

In certain embodiments, the bacteria-derived lipid composition and methods for making and using thereof include a lipid having a compound structure of:

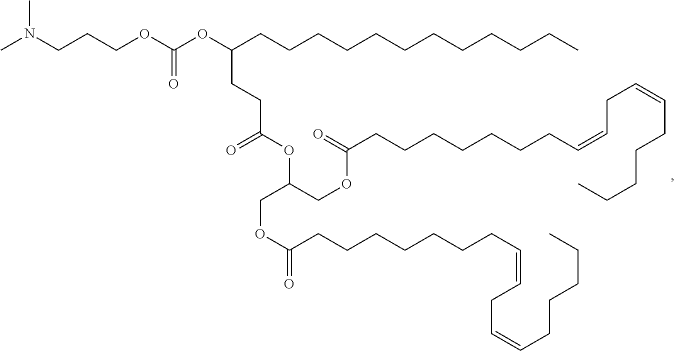 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof.

In certain embodiments, the bacteria-derived lipid composition and methods for making and using thereof include a lipid having a compound structure of:

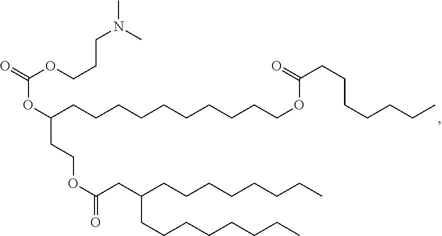 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

In some embodiments, the bacteria-derived lipid compositions described herein may include a ionizable lipid as described in, may be formulated as described in, or may comprise or be comprised by a composition as described in WO2016118724, WO2016118725, WO2016187531, WO2017176974, WO2018078053, WO2019027999, WO2019036030, WO2019089828, WO2019099501, WO2020072605, WO2020081938, WO2020118041, WO2020146805, or WO2020219876, each of which is incorporated by reference herein in its entirety.

### Other Lipids and Other Agents

The exogenous lipid may be a cell-penetrating agent, may be capable of increasing delivery of a polypeptide by the bacteria-derived lipid composition to a cell, and/or may be capable of increasing loading (e.g., loading efficiency or loading capacity) of a polypeptide. Further exemplary exogenous lipids include sterols and PEGylated lipids.

The bacteria-derived lipid compositions can include other components (e.g., lipids, e.g., sterols, e.g., cholesterol; or small molecules) to further alter the functional and structural characteristics of the bacteria-derived lipid composition. For example, the bacteria-derived lipid compositions can further include stabilizing molecules that increase the stability of the bacteria-derived lipid compositions (e.g., for at least one day at room temperature, and/or stable for at least one week at 4° C.).

In some embodiments, the bacteria-derived lipid composition further includes a sterol, e.g., sitosterol, sitostanol, β-sitosterol, 7α-hydroxycholesterol, pregnenolone, cholesterol (e.g., ovine cholesterol or cholesterol isolated from plants), stigmasterol, campesterol, fucosterol, or an analog (e.g., a glycoside, ester, or peptide) of any sterol. In some examples, the exogenous sterol is added to the preparation prior to step (b), e.g., mixed with extracted bacterial lipids prior to step (b). The exogenous sterol may be added to amount to, e.g., 1%, 5%, 10%, 20%, 30%, 40%, 50%, 60%, 70%, 80%, 90%, or more than 90% (w/w) of total lipids and sterols in the preparation.

In some embodiments, the sterol is cholesterol or sitosterol. In some instances, the bacteria-derived lipid compositions comprise a molar ratio of least 1%, 5%, 10%, 15%, 20%, 25%, 30%, 35%, 40%, 45%, 50%, 55%, 60%, or more than 60% sterol (e.g., cholesterol or sitosterol), e.g., 1%-10%, 10%-20%, 20%-30%, 30%-40%, 40%-50%, or 50%-60% sterol. In some embodiments, the bacteria-derived lipid composition comprises a molar ratio of about 35%-50% sterol (e.g., cholesterol or sitosterol), e.g., about 36%, 38.5%, 42.5%, or 46.5% sterol. In some embodiments, the bacteria-derived lipid composition comprises a molar ratio of about 20%-40% sterol.

In some embodiments, a bacteria-derived lipid composition that has been modified with a sterol has altered stability (e.g., increased stability) relative to a bacteria-derived lipid composition that has not been modified with a sterol. In some aspects, a bacteria-derived lipid composition that has been modified with a sterol has a greater rate of fusion with a membrane of a target cell relative to a bacteria-derived lipid composition that has not been modified with a sterol.

In some instances, the bacteria-derived lipid compositions comprise an exogenous lipid and an exogenous sterol.

In some embodiments, the bacteria-derived lipid composition further includes a PEGylated lipid. Polyethylene glycol (PEG) length can vary from 1 kDa to 10 kDa; in some aspects, PEG having a length of 2 kDa is used. In some embodiments, the PEGylated lipid is C14-PEG2k, C18-PEG2k, or DMPE-PEG2k. In some instances, the bacteria-derived lipid compositions comprise at least 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.5%, 4%, 4.5%, 5%, 10%, 20%, 30%, 40%, 50%, or more than 50% PEGylated lipid (e.g., C14-PEG2k, C18-PEG2k, or DMPE-PEG2k), e.g., 0.1%-0.5%, 0.5%-1%, 1%-1.5%, 1.5%-2.5%, 2.5%-3.5%, 3.5%-5%, 5%-10%, 10%-20%, 20%-30%, 30%-40%, or 30%-50% PEGylated lipid, all in mol %. In some embodiments, the bacteria-derived lipid composition comprises about 0.1%-10% PEGylated lipid (e.g., C14-PEG2k, C18-PEG2k, or DMPE-PEG2k), e.g., about 1%-3% PEGylated lipid, e.g., about 1.5% or about 2.5% PEGylated lipid, all in mol %.

In some embodiments, a bacteria-derived lipid composition that has been modified with a PEGylated lipid has altered stability (e.g., increased stability) relative to a bacteria-derived lipid composition that has not been modified with a PEGylated lipid. In some embodiments, a bacteria-derived lipid composition that has been modified with a PEGylated lipid has altered particle size relative to a bacteria-derived lipid composition that has not been modified with a PEGylated lipid. In some embodiments, a bacteria-derived lipid composition that has been modified with a PEGylated lipid is less likely to be phagocytosed than a bacteria-derived lipid composition that has not been modified with a PEGylated lipid. The addition of PEGylated lipids can also affect stability in GI tract and enhance particle migration through mucus. PEG may be used as a method to attach targeting moieties.

In some embodiments, the bacteria-derived lipid composition comprises an ionizable lipid (e.g., C12-200 or MC3) and one or both of a sterol (e.g., cholesterol or sitosterol) and a PEGylated lipid (e.g., C14-PEG2k, C18-PEG2k, or DMPE-PEG2k). In embodiments, the bacteria-derived lipid composition comprises about 5%-50% bacteria-derived lipids (e.g., about 10%-20% bacteria-derived lipids, e.g., about 10%, 12.5%, 16%, or 20% bacteria-derived lipids); about 30%-75% ionizable lipids (e.g., about 35% or about 50% ionizable lipids); about 35%-50% sterol (e.g., about 36%, 38.5%, 42.5%, or 46.5% sterol); and about 0.1%-10% PEGylated lipid (e.g., about 1%-3% PEGylated lipid, e.g., about 1.5% or about 2.5% PEGylated lipid), all in mol %.

In some embodiments, the bacteria-derived lipid composition comprise a molar ratio of about 5%-60% bacteria-derived lipids (e.g., about 10%-20%, 20%-30%, 30%-40%, 40%-50%, or 50%-60% bacteria-derived lipids, e.g., about 10%, 12.5%, 16%, 20%, 30%, 40%, 50%, or 60% bacteria-derived lipids); about 25%-75% ionizable lipids (e.g., about 35% or about 50% ionizable lipids); about 10%-50% sterol (e.g., about 10%, 12.5%, 14%, 16%, 18%, 20%, 36%, 38.5%, 42.5%, or 46.5% sterol); and about 0.1%-10% PEGylated lipid (e.g., about 0.5%-5% PEGylated lipid, e.g., about 1%-3% PEGylated lipid, or about 1.5% or about 2.5% PEGylated lipid).

In some embodiments, the ionizable lipids, bacteria-derived lipids, sterol, and PEGylated lipid comprise about 25%-75%, about 20%-60%, about 10%-45%, and about 0.5%-5%, respectively, of the lipids in the bacteria-derived lipid composition.

In some embodiments, the ionizable lipids, bacteria-derived lipids, sterol, and PEGylated lipid comprise about 30%-75%, about 20%-50%, about 10%-45%, and about 1%-5%, respectively, of the lipids in the bacteria-derived lipid composition.

In some embodiments, the ionizable lipids, bacteria-derived lipids, sterol, and PEGylated lipid comprise about 35%-75%, about 20%-50%, about 10%-45%, and about 1%-5%, respectively, of the lipids in the bacteria-derived lipid composition.

In some embodiments, the ionizable lipids, bacteria-derived lipids, sterol, and PEGylated lipid are formulated at a molar ratio of about 35:50:12.5:2.5.

In some embodiments, the ionizable lipids, bacteria-derived lipids, sterol, and PEGylated lipid are formulated at a molar ratio of about 35:50:11.5:3.5.

In some embodiments, the ionizable lipids, bacteria-derived lipids, sterol, and PEGylated lipid are formulated at a molar ratio of about 35:20:42.5:2.5.

In some embodiments, a bacteria-derived lipid composition that has been modified with an ionizable lipid (and/or cationic lipid) and a sterol and/or a PEGylated lipid more efficiently encapsulates a negatively charged cargo (e.g., a nucleic acid) than a bacteria-derived lipid composition that has not been modified with an ionizable lipid (and/or cationic lipid) and a sterol and/or a PEGylated lipid. The bacteria-derived lipid composition may have an encapsulation efficiency for a cargo (e.g., a heterologous functional agent such as nucleic acid, e.g., RNA or DNA) that is at least 5%, 10%, 15%, 20%, 30%, 40%, 50%, 60%, 70%, 80%, 90%, 95%, 98%, 99%, or more than 99%, e.g., it may have an encapsulation efficiency of 5%-30%, 30%-50%, 50%-70%, 70%-80%, 80%-90%, 90%-95%, or 95%-100%.

Cell uptake of the bacteria-derived lipid compositions can be measured by a variety of methods known in the art. For example, the bacteria-derived lipid composition, or a component thereof, can be labelled with a marker (e.g., a fluorescent marker) that can be detected in isolated cells to confirm uptake.

In some embodiments, a bacteria-derived lipid composition provided herein comprises two or more different bacteria components, e.g., bacterial components derived from two or more different bacterial sources. In some embodiments, a bacteria-derived lipid composition provided herein comprises two or more different types of modification, e.g., different types and/or ratios of ionizable lipids, sterols, and/or PEGylated lipids.

In some instances, the organic solvent in which the lipid film is dissolved is dimethylformamide:methanol (DMF:MeOH). Alternatively, the organic solvent or solvent combination may be, e.g., acetonitrile, acetone, ethanol, methanol, dimethylformamide, tetrahydrofuran, 1-buthanol, dimethyl sulfoxide, acetonitrile:ethanol, acetonitrile:methanol, acetone:methanol, methyl tert-butyl ether:propanol, tetrahydrofuran:methanol, dimethyl sulfoxide:methanol, or dimethylformamide:methanol.

The aqueous phase may be any suitable solution, e.g., a citrate buffer (e.g., a citrate buffer having a pH of about 3.2), water, or phosphate-buffered saline (PBS). The aqueous phase may further comprise a heterologous functional agent (e.g., an agricultural agent or a therapeutic agent) or a small molecule.

The lipid solution and the aqueous phase may be mixed in the microfluidics device at any suitable ratio. In some examples, aqueous phase and the lipid solution are mixed at a 3:1 volumetric ratio.

The bacteria-derived lipid composition may optionally include additional agents, e.g., cell-penetrating agents, therapeutic agents, polynucleotides, polypeptides, or small molecules. The bacteria-derived lipid composition can carry or associate with additional agents in a variety of ways to enable delivery of the heterologous functional agent to a target cell, e.g., by encapsulating the heterologous functional agent, incorporation of the heterologous functional agent in the lipid bilayer structure, or association of the heterologous functional agent (e.g., by conjugation) with the surface of the lipid bilayer structure. The heterologous functional agent can be incorporated into the bacteria-derived lipid composition either in vivo or in vitro (e.g., in tissue culture, in cell culture, or synthetically incorporated).

### Zeta Potential

The bacteria-derived lipid composition comprising an ionizable lipid (e.g., C12-200 or MC3) and optionally a cationic lipid (e.g., DC-cholesterol or DOTAP) may have, e.g., a zeta potential of greater than −30 mV when in the absence of cargo, greater than −20 mV, greater than −5 mV, greater than 0 mV, or about 30 my when in the absence of cargo. In some examples, the bacteria-derived lipid composition has a negative zeta potential, e.g., a zeta potential of less than 0 mV, less than −10 mV, less than −20 mV, less than −30 mV, less than −40 mV, or less than −50 mV when in the absence of cargo. In some examples, the bacteria-derived lipid composition has a positive zeta potential, e.g., a zeta potential of greater than 0 mV, greater than 10 mV, greater than 20 mV, greater than 30 mV, greater than 40 mV, or greater than 50 mV when in the absence of cargo. In some examples, the bacteria-derived lipid composition has a zeta potential of about 0.

The zeta potential of the bacteria-derived lipid composition may be measured using any method known in the art. Zeta potentials are generally measured indirectly, e.g., calculated using theoretical models from the data obtained using methods and techniques known in the art, e.g., electrophoretic mobility or dynamic electrophoretic mobility. Electrophoretic mobility is typically measured using microelectrophoresis, electrophoretic light scattering, or tunable resistive pulse sensing. Electrophoretic light scattering is based on dynamic light scattering. Typically, zeta potentials are accessible from dynamic light scattering (DLS) measurements, also known as photon correlation spectroscopy or quasi-elastic light scattering.

### Bacterial EV-Markers

The bacterial components (e.g., bacterial lipids) in the bacteria-derived lipid composition and methods of making and using thereof may have a range of markers that identify the bacterial component as being produced. As used herein, the term “bacterial EV-marker” refers to a component that is naturally associated with a bacterium and incorporated into or onto the bacterial EV, such as a bacterial protein, a bacterial nucleic acid, a bacterial small molecule, a bacterial lipid, or a combination thereof.

### Loading of Agents

The bacteria-derived lipid composition can include a heterologous functional agent, e.g., a cell-penetrating agent and/or a heterologous agricultural agent (e.g., pesticidal agent, fertilizing agent, herbicidal agent, plant-modifying agent), a heterologous therapeutic agent (e.g., an antifungal agent, an antibacterial agent, a virucidal agent, an anti-viral agent, an insecticidal agent, a nematicidal agent, an antiparasitic agent, or an insect repellent)), such as those described herein,

The bacteria-derived lipid composition can carry or associate with such agents by a variety of means to enable delivery of the agent to a target organism (e.g., a target animal, plant, bacterium, or fungus), e.g., by encapsulating the agent, incorporation of the agent in the lipid bilayer structure, or association of the agent (e.g., by conjugation) with the surface of the lipid bilayer structure of the bacteria-derived lipid composition. In some instances, the heterologous functional agent (e.g., cell-penetrating agent) is included in the formulation made of the bacteria-derived lipid composition formulation, as described herein.

The heterologous functional agent can be incorporated or loaded into or onto the bacteria-derived lipid composition by any methods known in the art that allow association, directly or indirectly, between the bacteria-derived lipid composition and agent. The agent can be incorporated into the bacteria-derived lipid composition by an in vivo or in vitro (e.g., in tissue culture, or in cell culture), or both in vivo and in vitro methods.

In some instances, the bacteria-derived lipid composition is loaded in vitro. The heterologous functional agent may be loaded onto or into (e.g., may be encapsulated by) the bacteria-derived lipid composition using, but not limited to, physical, chemical, and/or biological methods (e.g., in tissue culture or in cell culture). For example, the agent may be introduced into the bacteria-derived lipid composition by one or more of electroporation, sonication, passive diffusion, stirring, lipid extraction, or extrusion. In some instances, the agent is incorporated into the bacteria-derived lipid composition using a microfluidic device, e.g., using a method in which lipids are provided in an organic phase, the agent is provided in an aqueous phase, and the organic and aqueous phases are combined in the microfluidics device to produce a bacteria-derived lipid composition comprising the heterologous functional agent. Loaded bacteria-derived lipid composition can be assessed to confirm the presence or level of the loaded agent using a variety of methods, such as HPLC (e.g., to assess small molecules), immunoblotting (e.g., to assess proteins); and/or quantitative PCR (e.g., to assess nucleotides). However, it should be appreciated by those skilled in the art that the loading of a heterologous functional agent of interest into bacteria-derived lipid composition is not limited to the above-illustrated methods.

In some instances, the heterologous functional agent can be conjugated to the bacteria-derived lipid composition, in which the agent is connected or joined, indirectly or directly, to the bacteria-derived lipid composition. For instance, one or more agents can be chemically linked to a bacteria-derived lipid composition, such that the one or more agents are joined (e.g., by covalent or ionic bonds) directly to the lipid bilayer of the bacteria-derived lipid composition. In some instances, the conjugation of various agents to the bacteria-derived lipid composition can be achieved by first mixing the one or more agents with an appropriate cross-linking agent (e.g., N-ethylcarbo-diimide (“EDC”), which is generally utilized as a carboxyl activating agent for amide bonding with primary amines and also reacts with phosphate groups) in a suitable solvent. After a period of incubation sufficient to allow the agent to attach to the cross-linking agent, the cross-linking agent/agent mixture can then be combined with the bacteria-derived lipid composition, and, after another period of incubation, subjected to a sucrose gradient (e.g., and 8, 30, 45, and 60% sucrose gradient) to separate the free agent and free bacteria-derived lipid composition from the agent conjugated to the bacteria-derived lipid composition. As part of combining the mixture with a sucrose gradient, and an accompanying centrifugation step, the bacteria-derived lipid composition conjugated to the agent is then seen as a band in the sucrose gradient, such that the conjugated bacteria-derived lipid composition can be collected, washed, and dissolved in a suitable solution for use.

In some instances, the bacteria-derived lipid composition is stably associated with the heterologous functional agent prior to and following delivery of the bacteria-derived lipid composition. In other instances, the bacteria-derived lipid composition is associated with the agent such that the agent becomes dissociated from the bacteria-derived lipid composition following delivery of the bacteria-derived lipid composition.

The bacteria-derived lipid composition can be loaded or the bacteria-derived lipid composition can be formulated with various concentrations of the heterologous functional agent, depending on the particular agent or use. For example, in some instances, the bacteria-derived lipid composition is loaded or formulated to include about 0.001, 0.01, 0.1, 1.0, 2, 3, 4, 5, 6, 7, 8, 9, 10, 15, 20, 30, 40, 50, 60, 70, 80, 90, or 95 (or any range between about 0.001 and 95) or more wt % of an agent. In some instances, the bacteria-derived lipid composition is loaded or formulated to include about 95, 90, 80, 70, 60, 50, 40, 30, 20, 15, 10, 9, 8, 7, 6, 5, 4, 3, 2, 1.0, 0.1, 0.01, 0.001 (or any range between about 95 and 0.001) or less wt % of an agent. For example, the bacteria-derived lipid composition can include about 0.001 to about 0.01 wt %, about 0.01 to about 0.1 wt %, about 0.1 to about 1 wt %, about 1 to about 5 wt %, or about 5 to about 10 wt %, about 10 to about 20 wt % of the agent. In some instances, the bacteria-derived lipid composition can be loaded or formulated with about 1, 5, 10, 50, 100, 200, or 500, 1,000, 2,000 (or any range between about 1 and 2,000) or more μg/ml of an agent. A bacteria-derived lipid composition can be loaded or formulated with about 2,000, 1,000, 500, 200, 100, 50, 10, 5, 1 (or any range between about 2,000 and 1) or less μg/ml of an agent.

In some instances, the bacteria-derived lipid composition is loaded or formulated to include at least 0.001 wt %, at least 0.01 wt %, at least 0.1 wt %, at least 1.0 wt %, at least 2 wt %, at least 3 wt %, at least 4 wt %, at least 5 wt %, at least 6 wt %, at least 7 wt %, at least 8 wt %, at least 9 wt %, at least 10 wt %, at least 15 wt %, at least 20 wt %, at least 30 wt %, at least 40 wt %, at least 50 wt %, at least 60 wt %, at least 70 wt %, at least 80 wt %, at least 90 wt %, or at least 95 wt % of the heterologous functional agent. In some instances, the bacteria-derived lipid composition can be loaded or formulated with at least 1 μg/ml, at least 5 μg/ml, at least 10 μg/ml, at least 50 μg/ml, at least 100 μg/ml, at least 200 μg/ml, at least 500 μg/ml, at least 1,000 μg/ml, at least 2,000 μg/ml of the agent.

In some instances, the bacteria-derived lipid composition is formulated with the heterologous functional agent by suspending the bacteria-derived lipid compositions in a solution comprising or consisting of the agent, e.g., by vigorous mixing. The agent (e.g., cell-penetrating agent, e.g., nucleic acids, enzyme, detergent, ionic, fluorous, or zwitterionic liquid, or ionizable lipid may comprise, e.g., less than 1% or at least 1%, 5%, 10%, 20%, 30%, 40%, 50%, 60%, 70%, 80%, 90%, or 100% of the solution.

### Formulations

### Agricultural Formulations

The bacteria-derived lipid composition described herein can be formulated into an agricultural composition.

To allow ease of application, handling, transportation, storage, and effective activity, the bacteria-derived lipid composition can be formulated with other substances. The bacteria-derived lipid composition can be formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra-low volume solutions. For further information on formulation types see “Catalogue of Pesticide Formulation Types and International Coding System” Technical Monograph n° 2, 5th Edition by CropLife International (2002).

The bacteria-derived lipid composition can be applied as aqueous suspensions or emulsions prepared from concentrated formulations of such agents. Such water-soluble, water-suspendable, or emulsifiable formulations are either solids, usually known as wettable powders, or water dispersible granules, or liquids usually known as emulsifiable concentrates, or aqueous suspensions. Wettable powders, which may be compacted to form water dispersible granules, comprise an intimate mixture of the bacteria-derived lipid composition, a carrier, and surfactants. The carrier is usually selected from among the attapulgite clays, the montmorillonite clays, the diatomaceous earths, or the purified silicates. Effective surfactants, including from about 0.5% to about 10% of the wettable powder, are found among sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates can comprise a suitable concentration of the bacteria-derived lipid composition, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are selected from conventional anionic and non-ionic surfactants.

Aqueous suspensions comprise suspensions of water-insoluble bacteria-derived lipid composition dispersed in an aqueous carrier at a concentration in the range from about 5% to about 50% by weight. Suspensions are prepared by finely grinding the composition and vigorously mixing it into a carrier comprised of water and surfactants. Ingredients, such as inorganic salts and synthetic or natural gums may also be added, to increase the density and viscosity of the aqueous carrier.

The bacteria-derived lipid composition may also be applied as granular compositions that are particularly useful for applications to the soil. Granular compositions usually contain from about 0.5% to about 10% by weight of the bacteria-derived lipid composition, dispersed in a carrier that comprises clay or a similar substance. Such compositions are usually prepared by dissolving the formulation in a suitable solvent and applying it to a granular carrier which has been pre-formed to the appropriate particle size, in the range of from about 0.5 to about 3 mm. Such compositions may also be formulated by making a dough or paste of the carrier and compound and crushing and drying to obtain the desired granular particle size.

Dusts containing the bacteria-derived lipid composition are prepared by intimately mixing a bacteria-derived lipid composition in powdered form with a suitable dusty agricultural carrier, such as kaolin clay, ground volcanic rock, and the like. Dusts can suitably contain from about 1% to about 10% of the packets. They can be applied as a seed dressing or as a foliage application with a dust blower machine.

It is equally practical to apply the present formulation in the form of a solution in an appropriate organic solvent, usually petroleum oil, such as the spray oils, which are widely used in agricultural chemistry.

The bacteria-derived lipid composition can also be applied in the form of an aerosol composition. In such compositions the packets are dissolved or dispersed in a carrier, which is a pressure-generating propellant mixture. The aerosol composition is packaged in a container from which the mixture is dispensed through an atomizing valve.

Another embodiment is an oil-in-water emulsion, wherein the emulsion comprises oily globules which are each provided with a lamellar liquid crystal coating and are dispersed in an aqueous phase, wherein each oily globule comprises at least one compound which is agriculturally active, and is individually coated with a monolamellar or oligolamellar layer including: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007. For ease of use, this embodiment will be referred to as “OIWE.”

Additionally, generally, when the molecules disclosed above are used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulfate; sodium dioctyl sulfosuccinate; alkyl phenol ethoxylates; and aliphatic alcohol ethoxylates.

A dispersing agent is a substance which adsorbs onto the surface of particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulfonates. For suspension concentrates, very good adsorption and stabilization are obtained using polyelectrolytes, such as sodium naphthalene sulfonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulfonates; sodium naphthalene sulfonate formaldehyde condensates; tristyrylphenol ethoxylate phosphate esters; aliphatic alcohol ethoxylates; alkyl ethoxylates; EO-PO (ethylene oxide-propylene oxide) block copolymers; and graft copolymers.

An emulsifying agent is a substance which stabilizes a suspension of droplets of one liquid phase in another liquid phase. Without the emulsifying agent, the two liquids would separate into two immiscible liquid phases. The most commonly used emulsifier blends contain alkylphenol or aliphatic alcohol with twelve or more ethylene oxide units and the oil-soluble calcium salt of dodecylbenzenesulfonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilize water-insoluble materials inside the hydrophobic part of the micelle. The types of surfactants usually used for solubilization are non-ionics, sorbitan monooleates, sorbitan monooleate ethoxylates, and methyl oleate esters.

Surfactants are sometimes used, either alone or with other additives such as mineral or vegetable oils as adjuvants to spray-tank mixes to improve the biological performance of the bacteria-derived lipid composition on the target. The types of surfactants used for bioenhancement depend generally on the nature and mode of action of the bacteria-derived lipid composition. However, they are often non-ionics such as: alkyl ethoxylates; linear aliphatic alcohol ethoxylates; aliphatic amine ethoxylates.

A carrier or diluent in an agricultural formulation is a material added to the bacteria-derived lipid composition to give a product of the required strength. Carriers are usually materials with high absorptive capacities, while diluents are usually materials with low absorptive capacities. Carriers and diluents are used in the formulation of dusts, wettable powders, granules, and water-dispersible granules.

Organic solvents are used mainly in the formulation of emulsifiable concentrates, oil-in-water emulsions, suspoemulsions, and ultra low volume formulations, and to a lesser extent, granular formulations. Sometimes mixtures of solvents are used. The first main groups of solvents are aliphatic paraffinic oils such as kerosene or refined paraffins. The second main group (and the most common) comprises the aromatic solvents such as xylene and higher molecular weight fractions of C9 and C10 aromatic solvents. Chlorinated hydrocarbons are useful as cosolvents to prevent crystallization of the bacteria-derived lipid composition when the formulation is emulsified into water. Alcohols are sometimes used as cosolvents to increase solvent power. Other solvents may include vegetable oils, seed oils, and esters of vegetable and seed oils.

Thickeners or gelling agents are used mainly in the formulation of suspension concentrates, emulsions, and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are not limited to, montmorillonite, bentonite, magnesium aluminum silicate, and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenan; alginates; methyl cellulose; sodium carboxymethyl cellulose (SCMC); hydroxyethyl cellulose (HEC). Other types of anti-settling agents are based on modified starches, polyacrylates, polyvinyl alcohol, and polyethylene oxide. Another good anti-settling agent is xanthan gum.

Microorganisms can cause spoilage of formulated products. Therefore preservation agents are used to eliminate or reduce their effect. Examples of such agents include, but are not limited to: propionic acid and its sodium salt; sorbic acid and its sodium or potassium salts; benzoic acid and its sodium salt; p-hydroxybenzoic acid sodium salt; methyl p-hydroxybenzoate; and 1,2-benzisothiazolin-3-one (BIT).

The presence of surfactants often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane, while the non-silicone anti-foam agents are water-insoluble oils, such as octanol and nonanol, or silica. In both cases, the function of the anti-foam agent is to displace the surfactant from the air-water interface.

“Green” agents (e.g., adjuvants, surfactants, solvents) can reduce the overall environmental footprint of crop protection formulations. Green agents are biodegradable and generally derived from natural and/or sustainable sources, e.g., plant and animal sources. Specific examples are: vegetable oils, seed oils, and esters thereof, also alkoxylated alkyl polyglucosides.

In some instances, the bacteria-derived lipid composition can be freeze-dried or lyophilized. See U.S. Pat. No. 4,311,712. The bacteria-derived lipid composition can later be reconstituted on contact with water or another liquid. Other components can be added to the lyophilized or reconstituted bacteria-derived lipid composition, for example, other heterologous functional agents, agriculturally acceptable carriers, or other materials in accordance with the formulations described herein.

Other optional features of the composition include carriers or delivery vehicles that protect the bacteria-derived lipid composition against UV and/or acidic conditions. In some instances, the delivery vehicle contains a pH buffer. In some instances, the composition is formulated to have a pH in the range of about 4.5 to about 9.0, including for example pH ranges of about any one of 5.0 to about 8.0, about 6.5 to about 7.5, or about 6.5 to about 7.0.

For further information on agricultural formulations, see “Chemistry and Technology of Agrochemical Formulations” edited by D. A. Knowles, copyright 1998 by Kluwer Academic Publishers. Also see “Insecticides in Agriculture and Environment-Retrospects and Prospects” by A. S. Perry, I. Yamamoto, I. Ishaaya, and R. Perry, copyright 1998 by Springer-Verlag.

### Pharmaceutical Formulations

The bacteria-derived lipid compositions are formulated into pharmaceutical compositions (i.e., a bacteria-derived lipid composition composition), e.g., for administration to an animal (e.g., a human). The pharmaceutical composition may be administered to an animal (e.g., human) with a pharmaceutically acceptable diluent, carrier, and/or excipient. Depending on the mode of administration and the dosage, the pharmaceutical composition of the methods described herein will be formulated into suitable pharmaceutical compositions to permit facile delivery. The single dose may be in a unit dose form as needed.

The bacteria-derived lipid composition may be formulated for e.g., oral administration, intravenous administration (e.g., injection or infusion), intramuscular, or subcutaneous administration to an animal. For injectable formulations, various effective pharmaceutical carriers are known in the art (See, e.g., Remington: The Science and Practice of Pharmacy, 22^{nd} ed., (2012) and ASHP Handbook on Injectable Drugs, 18^{th} ed., (2014)).

Suitable pharmaceutically acceptable carriers and excipients are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. The bacteria-derived lipid composition may be formulated according to conventional pharmaceutical practice. The concentration of the compound in the formulation will vary depending upon a number of factors, including the dosage of the active agent (e.g., bacteria-derived lipid compositions and nucleic acids) to be administered, and the route of administration.

For oral administration to an animal, the bacteria-derived lipid composition can be prepared in the form of an oral formulation. Formulations for oral use can include tablets, caplets, capsules, syrups, or oral liquid dosage forms containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like. Formulations for oral use may also be provided in unit dosage form as chewable tablets, non-chewable tablets, caplets, capsules (e.g., as hard gelatin capsules wherein the active ingredient is mixed with an inert solid diluent, or as soft gelatin capsules wherein the active ingredient is mixed with water or an oil medium). The compositions disclosed herein may also further include an immediate-release, extended release or delayed-release formulation.

For parenteral administration to an animal, the bacteria-derived lipid composition may be formulated in the form of liquid solutions or suspensions and administered by a parenteral route (e.g., subcutaneous, intravenous, or intramuscular). The pharmaceutical composition can be formulated for injection or infusion. Pharmaceutical compositions for parenteral administration can be formulated using a sterile solution or any pharmaceutically acceptable liquid as a vehicle. Pharmaceutically acceptable vehicles include, but are not limited to, sterile water, physiological saline, or cell culture media (e.g., Dulbecco's Modified Eagle Medium (DMEM), α-Modified Eagles Medium (α-MEM), and F-12 medium). Formulation methods are known in the art, see e.g., Gibson (ed.) Pharmaceutical Preformulation and Formulation (2nd ed.) Taylor & Francis Group, CRC Press (2009).

### Heterologous Functional Agents

The bacteria-derived lipid composition can further include a heterologous functional agent, such as a heterologous functional agent (e.g., a heterologous agricultural agent (e.g., pesticidal agent, fertilizing agent, herbicidal agent, plant-modifying agent) or a heterologous therapeutic agent (e.g., an antifungal agent, an antibacterial agent, a virucidal agent, an anti-viral agent, an insecticidal agent, a nematicidal agent, an antiparasitic agent, or an insect repellent)). For example, the bacteria-derived lipid composition may encapsulate the heterologous functional agent. Alternatively, the heterologous functional agent can be embedded on or conjugated to the surface of the bacteria-derived lipid composition. In some instances, the bacteria-derived lipid composition includes two or more (e.g., 2, 3, 4, 5, 6, 7, 8, 9, 10, or more than 10) different heterologous functional agents. Heterologous functional agents may be added at any step during the manufacturing process effective to introduce the agent into the bacteria-derived lipid composition.

In certain instances, the heterologous functional agent (e.g., a heterologous agricultural agent (e.g., pesticidal agent, fertilizing agent, herbicidal agent, plant-modifying agent, a heterologous nucleic acid, a heterologous polypeptide, or a heterologous small molecule) or a heterologous therapeutic agent (e.g., an antifungal agent, an antibacterial agent, a virucidal agent, an anti-viral agent, a nematicidal agent, an antiparasitic agent, or an insect repellent)) can be modified. For example, the modification can be a chemical modification, e.g., conjugation to a marker, e.g., fluorescent marker or a radioactive marker. In other examples, the modification can include conjugation or operational linkage to a moiety that enhances the stability, delivery, targeting, bioavailability, or half-life of the agent, e.g., a lipid, a glycan, a polymer (e.g., PEG), or a cation moiety.

Examples of heterologous functional agents are outlined below.

### Heterologous Agricultural Agents

The bacteria-derived lipid composition can include a heterologous agricultural agent (e.g., an agent that effects a plant or an organism that associates with a plant and can be loaded into a bacteria-derived lipid composition), such as a pesticidal agent, herbicidal agent, fertilizing agent, or a plant-modifying agent.

For example, in some instances, the bacteria-derived lipid composition may include a pesticidal agent. The pesticidal agent can be an antifungal agent, an antibacterial agent, an insecticidal agent, a molluscicidal agent, a nematicidal agent, a virucidal agent, or a combination thereof. The pesticidal agent can be a chemical agent, such as those well known in the art. Alternatively or additionally, the pesticidal agent can be a peptide, a polypeptide, a nucleic acid, a polynucleotide, or a small molecule. The pesticidal agent may be an agent that can decrease the fitness of a variety of plant pests or can be one that targets one or more specific target plant pests (e.g., a specific species or genus of plant pests).

In some instances, the bacteria-derived lipid composition may include one or more heterologous fertilizing agents. Examples of heterologous fertilizing agents include plant nutrients or plant growth regulators, such as those well known in the art. Alternatively, or additionally, the fertilizing agent can be a peptide, a polypeptide, a nucleic acid, or a polynucleotide that can increase the fitness of a plant symbiont. The fertilizing agent may be an agent that can increase the fitness of a variety of plants or plant symbionts or can be one that targets one or more specific target plants or plant symbionts (e.g., a specific species or genera of plants or plant symbionts).

In other instances, the bacteria-derived lipid composition may include one or more heterologous plant-modifying agents. In some instances, the plant-modifying agent can include a peptide or a nucleic acid.

### Antibacterial Agents

The bacteria-derived lipid composition described herein can further include an antibacterial agent. In some instances, the bacteria-derived lipid composition includes two or more (e.g., 2, 3, 4, 5, 6, 7, 8, 9, 10, or more than 10) different antibacterial agents. For example, the antibacterial agent can decrease the fitness of (e.g., decrease growth or kill) a bacterial plant pest (e.g., a bacterial plant pathogen). A bacteria-derived lipid composition including an antibiotic can be contacted with a target pest, or plant infested thereof, in an amount and for a time sufficient to: (a) reach a target level (e.g., a predetermined or threshold level) of antibiotic concentration inside or on the target pest; and (b) decrease fitness of the target pest. The antibacterials may be formulated in a bacteria-derived lipid composition for any of the methods described herein, and in certain instances, may be associated with the bacteria-derived lipid composition.

As used herein, the term “antibacterial agent” refers to a material that kills or inhibits the growth, proliferation, division, reproduction, or spread of bacteria, such as phytopathogenic bacteria, and includes bactericidal (e.g., disinfectant compounds, antiseptic compounds, or antibiotics) or bacteriostatic agents (e.g., compounds or antibiotics). Bactericidal antibiotics kill bacteria, while bacteriostatic antibiotics only slow their growth or reproduction.

Bactericides can include disinfectants, antiseptics, or antibiotics. The most used disinfectants can comprise: active chlorine (i.e., hypochlorites (e.g., sodium hypochlorite), chloramines, dichloroisocyanurate and trichloroisocyanurate, wet chlorine, chlorine dioxide etc.), active oxygen (peroxides, such as peracetic acid, potassium persulfate, sodium perborate, sodium percarbonate and urea perhydrate), iodine (iodpovidone (povidone-iodine, Betadine), Lugol's solution, iodine tincture, iodinated nonionic surfactants), concentrated alcohols (mainly ethanol, 1-propanol, called also n-propanol and 2-propanol, called isopropanol and mixtures thereof; further, 2-phenoxyethanol and 1- and 2-phenoxypropanols are used), phenolic substances (such as phenol (also called carbolic acid), cresols (called Lysole in combination with liquid potassium soaps), halogenated (chlorinated, brominated) phenols, such as hexachlorophene, triclosan, trichlorophenol, tribromophenol, pentachlorophenol, Dibromol and salts thereof), cationic surfactants, such as some quaternary ammonium cations (such as benzalkonium chloride, cetyl trimethylammonium bromide or chloride, didecyldimethylammonium chloride, cetylpyridinium chloride, benzethonium chloride) and others, non-quaternary compounds, such as chlorhexidine, glucoprotamine, octenidine dihydrochloride etc.), strong oxidizers, such as ozone and permanganate solutions; heavy metals and their salts, such as colloidal silver, silver nitrate, mercury chloride, phenylmercury salts, copper sulfate, copper oxide-chloride, copper hydroxide, copper octanoate, copper oxychloride sulfate, copper sulfate, copper sulfate pentahydrate, etc. Heavy metals and their salts are the most toxic, and environment-hazardous bactericides and therefore, their use is strongly oppressed or canceled; further, also properly concentrated strong acids (phosphoric, nitric, sulfuric, amidosulfuric, toluenesulfonic acids) and alkalis (sodium, potassium, calcium hydroxides).

As antiseptics (i.e., germicide agents that can be used on human or animal body, skin, mucoses, wounds and the like), few of the above mentioned disinfectants can be used, under proper conditions (mainly concentration, pH, temperature and toxicity toward man/animal). Among them, important are: properly diluted chlorine preparations (i.e., Daquin's solution, 0.5% sodium or potassium hypochlorite solution, pH-adjusted to pH 7-8, or 0.5-1% solution of sodium benzenesulfochloramide (chloramine B)), some iodine preparations, such as iodopovidone in various galenics (ointment, solutions, wound plasters), in the past also Lugol's solution, peroxides as urea perhydrate solutions and pH-buffered 0.1-0.25% peracetic acid solutions, alcohols with or without antiseptic additives, used mainly for skin antisepsis, weak organic acids such as sorbic acid, benzoic acid, lactic acid and salicylic acid some phenolic compounds, such as hexachlorophene, triclosan and Dibromol, and cation-active compounds, such as 0.05-0.5% benzalkonium, 0.5-4% chlorhexidine, 0.1-2% octenidine solutions.

The bacteria-derived lipid composition may include an antibiotic. Any antibiotic known in the art may be used. Antibiotics are commonly classified based on their mechanism of action, chemical structure, or spectrum of activity.

The antibiotic described herein may target any bacterial function or growth processes and may be either bacteriostatic (e.g., slow or prevent bacterial growth) or bactericidal (e.g., kill bacteria). In some instances, the antibiotic is a bactericidal antibiotic. In some instances, the bactericidal antibiotic is one that targets the bacterial cell wall (e.g., penicillins and cephalosporins); one that targets the cell membrane (e.g., polymyxins); or one that inhibits essential bacterial enzymes (e.g., rifamycins, lipiarmycins, quinolones, and sulfonamides). In some instances, the bactericidal antibiotic is an aminoglycoside (e.g., kasugamycin). In some instances, the antibiotic is a bacteriostatic antibiotic. In some instances the bacteriostatic antibiotic targets protein synthesis (e.g., macrolides, lincosamides, and tetracyclines). Additional classes of antibiotics that may be used herein include cyclic lipopeptides (such as daptomycin), glycylcyclines (such as tigecycline), oxazolidinones (such as linezolid), or lipiarmycins (such as fidaxomicin). Examples of antibiotics include rifampicin, ciprofloxacin, doxycycline, ampicillin, and polymyxin B. The antibiotic described herein may have any level of target specificity (e.g., narrow- or broad-spectrum). In some instances, the antibiotic is a narrow-spectrum antibiotic, and thus targets specific types of bacteria, such as gram-negative or gram-positive bacteria. Alternatively, the antibiotic may be a broad-spectrum antibiotic that targets a wide range of bacteria.

Other non-limiting examples of antibiotics are found in Table 1 of WO 2021/041301, which is incorporated herein by reference in its entirety. One skilled in the art will appreciate that a suitable concentration of each antibiotic in the composition depends on factors such as efficacy, stability of the antibiotic, number of distinct antibiotics, the formulation, and methods of application of the composition.

### Antifungal Agents

The bacteria-derived lipid composition can further include an antifungal agent. In some instances, the bacteria-derived lipid composition includes two or more (e.g., 2, 3, 4, 5, 6, 7, 8, 9, 10, or more than 10) different antifungal agents. For example, the antifungal agent can decrease the fitness of (e.g., decrease growth or kill) a fungal plant pest. A bacteria-derived lipid composition including an antifungal can be contacted with a target fungal pest, or plant infested therewith, in an amount and for a time sufficient to: (a) reach a target level (e.g., a predetermined or threshold level) of antibiotic concentration inside or on the target fungus; and (b) decrease fitness of the target fungus. The antifungals may be formulated in a bacteria-derived lipid composition for any of the methods described herein, and in certain instances, may be associated with the bacteria-derived lipid composition.

As used herein, the term “fungicide” or “antifungal agent” refers to a substance that kills or inhibits the growth, proliferation, division, reproduction, or spread of fungi, such as phytopathogenic fungi. Many different types of antifungal agent have been produced commercially. Non limiting examples of antifungal agents include: azoxystrobin, mancozeb, prothioconazole, folpet, tebuconazole, difenoconazole, captan, bupirimate, or fosetyl-AI. Further exemplary fungicides include, but are not limited to, strobilurins, azoxystrobin, dimoxystrobin, enestroburin, fluoxastrobin, kresoxim-methyl, metominostrobin, picoxystrobin, pyraclostrobin, trifloxystrobin, orysastrobin, carboxamides, carboxanilides, benalaxyl, benalaxyl-M, benodanil, carboxin, mebenil, mepronil, fenfuram, fenhexamid, flutolanil, furalaxyl, furcarbanil, furametpyr, metalaxyl, metalaxyl-M (mefenoxam), methfuroxam, metsulfovax, ofurace, oxadixyl, oxycarboxin, penthiopyrad, pyracarbolid, salicylanilide, tecloftalam, thifluzamide, tiadinil, N-biphenylamides, bixafen, boscalid, carboxylic acid morpholides, dimethomorph, flumorph, benzamides, flumetover, fluopicolid (picobenzamid), zoxamid, carboxamides, carpropamid, diclocymet, mandipropamid, silthiofam, azoles, triazoles, bitertanol, bromuconazole, cyproconazole, difenoconazole, diniconazole, enilconazole, epoxiconazole, fenbuconazole, flusilazol, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole, Imidazoles, cyazofamid, imazalil, pefurazoate, prochloraz, triflumizole, benzimidazoles, benomyl, carbendazim, fuberidazole, thiabendazole, ethaboxam, etridiazole, hymexazol, nitrogen-containing heterocyclyl compounds, pyridines, fuazinam, pyrifenox, pyrimidines, bupirimate, cyprodinil, ferimzone, fenarimol, mepanipyrim, nuarimol, pyrimethanil, piperazines, triforine, pyrroles, fludioxonil, fenpiclonil, morpholines, aldimorph, dodemorph, fenpropimorph, tridemorph, dicarboximides, iprodione, procymidone, vinclozolin, acibenzolar-S-methyl, anilazine, captan, captafol, dazomet, diclomezin, fenoxanil, folpet, fenpropidin, famoxadon, fenamidon, octhilinone, probenazole, proquinazid, pyroquilon, quinoxyfen, tricyclazole, carbamates, dithiocarbamates, ferbam, mancozeb, maneb, metiram, metam, propineb, thiram, zineb, ziram, diethofencarb, flubenthiavalicarb, iprovalicarb, propamocarb, guanidines, dodine, iminoctadine, guazatine, kasugamycin, polyoxins, streptomycin, validamycin A, organometallic compounds, fentin salts, sulfur-containing heterocyclyl compounds, isoprothiolane, dithianone, organophosphorous compounds, edifenphos, fosetyl, fosetyl-aluminum, iprobenfos, pyrazophos, tolclofos-methyl, Organochlorine compounds, thiophanate-methyl, chlorothalonil, dichlofluanid, tolylfluanid, flusulfamide, phthalide, hexachlorobenzene, pencycuron, quintozene, nitrophenyl derivatives, binapacryl, dinocap, dinobuton, spiroxamine, cyflufenamid, cymoxanil, metrafenon, N-2-cyanophenyl-3,4-dichloroisothiazol-5-carboxamide (isotianil), N-(3′,4′,5′-trifluorobiphenyl-2-yl)-3-difluoromethyl-1-methylpyrazole-4-carboxamide, 3-[5-(4-chlorophenyl)-2,3-dimethylisoxazolidin-3-yl]-pyridine, N-(3′,4′-dichloro-4-fluorobiphenyl-2-yl)-3-difluoromethyl-1-methylpyrazol-e-4-carboxamide, 5-chloro-7-(4-methylpiperidin-1-yl)-6-(2,4,6-trifluorophenyl)-[1,2,4]tria-zolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazo-le-1-sulfonamide, methyl-(2-chloro-5-[1-(3-methylbenzyloxyimino)-ethyl]benzyl)carbamate, methyl-(2-chloro-5-[1-(6-methylpyridin-2-ylmethoxy-imino)ethyl]benzyl)carbamate, methyl 3-(4-chlorophenyl)-3-(2-isopropoxycarbonylamino-3-methylbutyryl-amino)propionate, 4-fluorophenyl N-(1-(1-(4-cyanophenyl)ethanesulfonyl)but-2-yl)carbamate, N-(2-(4-[3-(4-chlorophenyl)prop-2-ynyloxy]-3-methoxyphenyl)ethyl)-2-metha-nesulfonylamino-3-methylbutyramide, N-(2-(4-[3-(4-chlorophenyl)prop-2-ynyloxy]-3-methoxyphenyl)ethyl)-2-ethan-esulfonylamino-3-methylbutyramide, N-(4′-bromobiphenyl-2-yl)-4-difluoromethyl-2-methylthiazol-5-carboxamide, N-(4′-trifluoromethylbiphenyl-2-yl)-4-difluoromethyl-2-methylthiazol-5-carboxamide, N-(4′-chloro-3′-fluorobiphenyl-2-yl)-4-difluoromethyl-2-methylt-hiazol-5-carboxamide, or methyl 2-(ortho-((2,5-dimethylphenyloxy-methylene)phenyl)-3-methoxyacrylate. One skilled in the art will appreciate that a suitable concentration of each antifungal in the composition depends on factors such as efficacy, stability of the antifungal, number of distinct antifungals, the formulation, and methods of application of the composition.

### Insecticides

The bacteria-derived lipid composition can further include an insecticide. In some instances, the bacteria-derived lipid compositions include two or more (e.g., 2, 3, 4, 5, 6, 7, 8, 9, 10, or more than 10) different insecticide agents. For example, the insecticide can decrease the fitness of (e.g., decrease growth or kill) an insect plant pest. A bacteria-derived lipid composition including an insecticide can be contacted with a target insect pest, or plant infested therewith, in an amount and for a time sufficient to: (a) reach a target level (e.g., a predetermined or threshold level) of insecticide concentration inside or on the target insect; and (b) decrease fitness of the target insect. The insecticides may be formulated in a bacteria-derived lipid composition for any of the methods described herein, and in certain instances, may be associated with the bacteria-derived lipid composition.

As used herein, the term “insecticide” or “insecticidal agent” refers to a substance that kills or inhibits the growth, proliferation, reproduction, or spread of insects, such as agricultural insect pests. Non limiting examples of insecticides are shown in Table 2 of WO 2021/041301, which is incorporated herein by reference in its entirety. Additional non-limiting examples of suitable insecticides include biologics, hormones or pheromones such as azadirachtin, Bacillus species, Beauveria species, codlemone, Metarrhizium species, Paecilomyces species, thuringiensis, and Verticillium species, and active compounds having unknown or non-specified mechanisms of action such as fumigants (such as aluminum phosphide, methyl bromide and sulphuryl fluoride) and selective feeding inhibitors (such as cryolite, flonicamid and pymetrozine). One skilled in the art will appreciate that a suitable concentration of each insecticide in the composition depends on factors such as efficacy, stability of the insecticide, number of distinct insecticides, the formulation, and methods of application of the composition.

### Nematicide

The bacteria-derived lipid composition can further include a nematicide. In some instances, the bacteria-derived lipid composition includes two or more (e.g., 2, 3, 4, 5, 6, 7, 8, 9, 10, or more than 10) different nematicides. For example, the nematicide can decrease the fitness of (e.g., decrease growth or kill) a nematode plant pest. A bacteria-derived lipid composition including a nematicide can be contacted with a target nematode pest, or plant infested therewith, in an amount and for a time sufficient to: (a) reach a target level (e.g., a predetermined or threshold level) of nematicide concentration inside or on the target nematode; and (b) decrease fitness of the target nematode. The nematicides may be formulated in a bacteria-derived lipid composition for any of the methods described herein, and in certain instances, may be associated with the bacteria-derived lipid composition.

As used herein, the term “nematicide” or “nematicidal agent” refers to a substance that kills or inhibits the growth, proliferation, reproduction, or spread of nematodes, such as agricultural nematode pests. Non limiting examples of nematicides are shown in Table 3 of WO 2021/041301, which is incorporated herein by reference in its entirety. One skilled in the art will appreciate that a suitable concentration of each nematicide in the composition depends on factors such as efficacy, stability of the nematicide, number of distinct nematicides, the formulation, and methods of application of the composition.

### Molluscicide

The bacteria-derived lipid composition can further include a molluscicide. In some instances, the bacteria-derived lipid composition includes two or more (e.g., 2, 3, 4, 5, 6, 7, 8, 9, 10, or more than 10) different molluscicides. For example, the molluscicide can decrease the fitness of (e.g., decrease growth or kill) a mollusk plant pest. A bacteria-derived lipid composition including a molluscicide can be contacted with a target mollusk pest, or plant infested therewith, in an amount and for a time sufficient to: (a) reach a target level (e.g., a predetermined or threshold level) of molluscicide concentration inside or on the target mollusk; and (b) decrease fitness of the target mollusk. The molluscicides may be formulated in a bacteria-derived lipid composition for any of the methods described herein, and in certain instances, may be associated with the bacteria-derived lipid composition.

As used herein, the term “molluscicide” or “molluscicidal agent” refers to a substance that kills or inhibits the growth, proliferation, reproduction, or spread of mollusks, such as agricultural mollusk pests. A number of chemicals can be employed as a molluscicide, including metal salts such as iron(III) phosphate, aluminium sulfate, and ferric sodium EDTA, [3][4], metaldehyde, methiocarb, or acetylcholinesterase inhibitors. One skilled in the art will appreciate that a suitable concentration of each molluscicide in the composition depends on factors such as efficacy, stability of the molluscicide, number of distinct molluscicides, the formulation, and methods of application of the composition.

### Virucides

The bacteria-derived lipid composition can further include a virucide. In some instances, the bacteria-derived lipid composition includes two or more (e.g., 2, 3, 4, 5, 6, 7, 8, 9, 10, or more than 10) different virucides. For example, the virucide can decrease the fitness of (e.g., decrease or eliminate) a viral plant pathogen. A bacteria-derived lipid composition including a virucide as described herein can be contacted with a target virus, or plant infested therewith, in an amount and for a time sufficient to: (a) reach a target level (e.g., a predetermined or threshold level) of virucide concentration; and (b) decrease or eliminate the target virus. The virucides described herein may be formulated in a bacteria-derived lipid composition for any of the methods described herein, and in certain instances, may be associated with the bacteria-derived lipid composition.

As used herein, the term “virucide” or “antiviral” refers to a substance that kills or inhibits the growth, proliferation, reproduction, development, or spread of viruses, such as agricultural virus pathogens. A number of agents can be employed as a virucide, including chemicals or biological agents (e.g., nucleic acids, e.g., dsRNA). One skilled in the art will appreciate that a suitable concentration of each virucide in the composition depends on factors such as efficacy, stability of the virucide, number of distinct virucides, the formulation, and methods of application of the composition.

### Herbicides

The bacteria-derived lipid composition can further include one or more (e.g., 1, 2, 3, 4, 5, 6, 7, 8, 9, 10, or more than 10) herbicide. For example, the herbicide can decrease the fitness of (e.g., decrease or eliminate) a weed. A bacteria-derived lipid composition including an herbicide can be contacted with a target weed in an amount and for a time sufficient to: (a) reach a target level (e.g., a predetermined or threshold level) of herbicide concentration on the plant and (b) decrease the fitness of the weed. The herbicides may be formulated in a bacteria-derived lipid composition for any of the methods described herein, and in certain instances, may be associated with the bacteria-derived lipid composition.

As used herein, the term “herbicide” refers to a substance that kills or inhibits the growth, proliferation, reproduction, or spread of weeds. A number of chemicals can be employed as a herbicides, including Glufosinate, Propaquizafop, Metamitron, Metazachlor, Pendimethalin, Flufenacet, Diflufenican, Clomazone, Nicosulfuron, Mesotrione, Pinoxaden, Sulcotrione, Prosulfocarb, Sulfentrazone, Bifenox, Quinmerac, Triallate, Terbuthylazine, Atrazine, Oxyfluorfen, Diuron, Trifluralin, or Chlorotoluron. Further examples of herbicides include, but are not limited to, benzoic acid herbicides, such as dicamba esters, phenoxyalkanoic acid herbicides, such as 2,4-D, MCPA and 2,4-DB esters, aryloxyphenoxypropionic acid herbicides, such as clodinafop, cyhalofop, fenoxaprop, fluazifop, haloxyfop, and quizalofop esters, pyridinecarboxylic acid herbicides, such as aminopyralid, picloram, and clopyralid esters, pyrimidinecarboxylic acid herbicides, such as aminocyclopyrachlor esters, pyridyloxyalkanoic acid herbicides, such as fluoroxypyr and triclopyr esters, and hydroxybenzonitrile herbicides, such as bromoxynil and ioxynil esters, esters of the arylpyridine carboxylic acids, and arylpyrimidine carboxylic acids of the generic structures disclosed in U.S. Pat. Nos. 7,314,849, 7,300,907, and 7,642,220, each of which is incorporated by reference herein in its entirety. In certain embodiments, the herbicide can be selected from the group consisting of 2,4-D, 2,4-DB, acetochlor, acifluorfen, alachlor, ametryn, amitrole, asulam, atrazine, azafenidin, benefin, bensulfuron, bensulide, bentazon, bromacil, bromoxynil, butylate, carfentrazone, chloramben, chlorimuron, chlorproham, chlorsulfuron, clethodim, clomazone, clopyralid, cloransulam, cyanazine, cycloate, DCPA, desmedipham, dichlobenil, diclofop, diclosulam, diethatyl, difenzoquat, diflufenzopyr, dimethenamid-p, diquat, diuron, DSMA, endothall, EPTC, ethalfluralin, ethametsulfuron, ethofumesate, fenoxaprop, fluazifop-P, flucarbazone, flufenacet, flumetsulam, flumiclorac, flumioxazin, fluometuron, fluroxypyr, fluthiacet, fomesafen, foramsulfuron, glufosinate, glyphosate, halosulfuron, haloxyfop, hexazinone, imazamethabenz, imazamox, imazapic, imazaquin, imazethapyr, isoxaben, isoxaflutole, lactofen, linuron, MCPA, MCPB, mesotrione, methazole, metolachlor-s, metribuzin, metsulfuron, molinate, MSMA, napropamide, naptalam, nicosulfuron, norflurazon, oryzalin, oxadiazon, oxasulfuron, oxyfluorfen, paraquat, pebulate, pelargonic acid, pendimethalin, phenmedipham, picloram, primisulfuron, prodiamine, prometryn, pronamide, propachlor, propanil, prosulfuron, pyrazon, pyridate, pyrithiobac, quinclorac, quizalofop, rimsulfuron, sethoxydim, siduron, simazine, sulfentrazone, sulfometuron, sulfosulfuron, tebuthiuron, terbacil, thiazopyr, thifensulfuron, thiobencarb, tralkoxydim, triallate, triasulfuron, tribenuron, triclopyr, trifluralin, triflusulfuron, vernolate. One skilled in the art will appreciate that a suitable concentration of each herbicide in the composition depends on factors such as efficacy, stability of the herbicide, number of distinct herbicides, the formulation, and methods of application of the composition.

### Repellents

The bacteria-derived lipid composition can further include a repellent. In some instances, the bacteria-derived lipid composition includes two or more (e.g., 2, 3, 4, 5, 6, 7, 8, 9, 10, or more than 10) different repellents. For example, the repellent can repel any of the pests described herein (e.g., insects, nematodes, or mollusks); microorganisms (e.g., phytopathogens or endophytes, such as bacteria, fungi, or viruses); or weeds. A bacteria-derived lipid composition including a repellent can be contacted with a target plant, or plant infested therewith, in an amount and for a time sufficient to: (a) reach a target level (e.g., a predetermined or threshold level) of repellent concentration; and (b) decrease the levels of the pest on the plant relative to an untreated plant. The repellent may be formulated in a bacteria-derived lipid composition for any of the methods described herein, and in certain instances, may be associated with the bacteria-derived lipid composition.

In some instances, the repellent is an insect repellent. Some examples of well-known insect repellents include: benzil; benzyl benzoate; 2,3,4,5-bis(butyl-2-ene)tetrahydrofurfural (MGK Repellent 11); butoxypolypropylene glycol; N-butylacetanilide; normal-butyl-6,6-dimethyl-5,6-dihydro-1,4-pyrone-2-carboxylate (Indalone); dibutyl adipate; dibutyl phthalate; di-normal-butyl succinate (Tabatrex); N,N-diethyl-meta-toluamide (DEET); dimethyl carbate (endo,endo)-dimethyl bicyclo[2.2.1]hept-5-ene-2,3-dicarboxylate); dimethyl phthalate; 2-ethyl-2-butyl-1,3-propanediol; 2-ethyl-1,3-hexanediol (Rutgers 612); di-normal-propyl isocinchomeronate (MGK Repellent 326); 2-phenylcyclohexanol; p-methane-3,8-diol, and normal-propyl N,N-diethylsuccinamate. Other repellents include citronella oil, dimethyl phthalate, normal-butylmesityl oxide oxalate and 2-ethyl hexanediol-1,3 (See, Kirk-Othmer Encyclopedia of Chemical Technology, 2nd Ed., Vol. 11: 724-728; and The Condensed Chemical Dictionary, 8th Ed., p 756).

An insect repellent may be a synthetic or nonsynthetic insect repellent. Examples of synthetic insect repellents include methyl anthranilate and other anthranilate-based insect repellents, benzaldehyde, DEET (N,N-diethyl-m-toluamide), dimethyl carbate, dimethyl phthalate, icaridin (i.e., picaridin, Bayrepel, and KBR 3023), indalone (e.g., as used in a “6-2-2” mixture (60% Dimethyl phthalate, 20% Indalone, 20% Ethylhexanediol), IR3535 (3-[N-Butyl-N-acetyl]-aminopropionic acid, ethyl ester), metofluthrin, permethrin, SS220, or tricyclodecenyl allyl ether. Examples of natural insect repellents include beautyberry (Callicarpa) leaves, birch tree bark, bog myrtle (Myrica Gale), catnip oil (e.g., nepetalactone), citronella oil, essential oil of the lemon eucalyptus (Corymbia citriodora; e.g., p-menthane-3,8-diol (PMD)), neem oil, lemongrass, tea tree oil from the leaves of Melaleuca alternifolia, tobacco, or extracts thereof.

### Fertilizing Agents

The bacteria-derived lipid composition can further include a heterologous fertilizing agent. In some instances, the heterologous fertilizing agent is associated with the bacteria-derived lipid composition. For example, a bacteria-derived lipid composition may encapsulate the heterologous fertilizing agent. Additionally, or alternatively, the heterologous fertilizing agent can be embedded on or conjugated to the surface of the bacteria-derived lipid composition.

Examples of heterologous fertilizing agents include plant nutrients or plant growth regulators, such as those well known in the art. Alternatively, or additionally, the fertilizing agent can be a peptide, a polypeptide, a nucleic acid, or a polynucleotide that can increase the fitness of a plant symbiont. The fertilizing agent may be an agent that can increase the fitness of a variety of plants or plant symbionts or can be one that targets one or more specific target plants or plant symbionts (e.g., a specific species or genera of plants or plant symbionts).

In some instances, the heterologous fertilizing agent can be modified. For example, the modification can be a chemical modification, e.g., conjugation to a marker, e.g., fluorescent marker or a radioactive marker. In other examples, the modification can include conjugation or operational linkage to a moiety that enhances the stability, delivery, targeting, bioavailability, or half-life of the agent, e.g., a lipid, a glycan, a polymer (e.g., PEG), a cation moiety.

In some instances, the heterologous fertilizing agent includes any material of natural or synthetic origin that is applied to soils or to plant tissues to supply one or more plant nutrients essential to the growth of plants. The plant nutrient may include a macronutrient, micronutrient, or a combination thereof. Plant macronutrients include nitrogen, phosphorus, potassium, calcium, magnesium, and/or sulfur. Plant micronutrients include copper, iron, manganese, molybdenum, zinc, boron, silicon, cobalt, and/or vanadium. Examples of plant nutrient fertilizers include a nitrogen fertilizer including, but not limited to urea, ammonium nitrate, ammonium sulfate, non-pressure nitrogen solutions, aqua ammonia, anhydrous ammonia, ammonium thiosulfate, sulfur-coated urea, urea-formaldehydes, IBDU, polymer-coated urea, calcium nitrate, ureaform, or methylene urea, phosphorous fertilizers such as diammonium phosphate, monoammonium phosphate, ammonium polyphosphate, concentrated superphosphate and triple superphosphate, or potassium fertilizers such as potassium chloride, potassium sulfate, potassium-magnesium sulfate, potassium nitrate. Such compositions can exist as free salts or ions within the composition. Fertilizers may be designated by the content of one or more of its components, such as nitrogen, phosphorous, or potassium. The content of these elements in a fertilizer may be indicated by the N—P—K value (where N=nitrogen content by weight percentage, P=phosphorous content by weight percentage, and K=potassium content by weight percentage).

Inorganic fertilizers, on the other hand, are manufactured from non-living materials and include, for example, ammonium nitrate, ammonium sulfate, urea, potassium chloride, potash, ammonium phosphate, anhydrous ammonia, and other phosphate salts. Inorganic fertilizers are readily commercially available and contain nutrients in soluble form that are immediately available to the plant. Inorganic fertilizers are generally inexpensive, having a low unit cost for the desired element. One skilled in the art will appreciate that the exact amount of a given element in a fertilizing agent may be calculated and administered to the plant or soil.

Fertilizers may be further classified as either organic fertilizers or inorganic fertilizers. Organic fertilizers include fertilizers having a molecular skeleton with a carbon backbone, such as in compositions derived from living matter. Organic fertilizers are made from materials derived from living things. Animal manures, compost, bonemeal, feather meal, and blood meal are examples of common organic fertilizers. Organic fertilizers, on the other hand, are typically not immediately available to plants and require soil microorganisms to break the fertilizer components down into simpler structures prior to use by the plants. In addition, organic fertilizers may not only elicit a plant growth response as observed with common inorganic fertilizers, but natural organic fertilizers may also stimulate soil microbial population growth and activities. Increased soil microbial population (e.g., plant symbionts) may have significant beneficial effects on the physical and chemical properties of the soil, as well as increasing disease and pest resistance.

In one aspect, a bacteria-derived lipid composition including a plant nutrient can be contacted with the plant in an amount and for a time sufficient to: (a) reach a target level (e.g., a predetermined or threshold level) of plant nutrient concentration inside or on the plant, and (b) increase the fitness of the plant relative to an untreated plant.

In another aspect, a bacteria-derived lipid composition including a plant nutrient can be contacted with the plant symbiont in an amount and for a time sufficient to: (a) reach a target level (e.g., a predetermined or threshold level) of plant nutrient concentration inside or on the plant symbiont (e.g., a bacteria or fungal endosymbiont), and (b) increase the fitness of the plant symbiont relative to an untreated plant symbiont.

The heterologous fertilizing agent may include a plant growth regulator. Exemplary plant growth regulators include auxins, cytokinins, gibberellins, and abscisic acid. In some instances, the plant growth regulator is abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole. Other plant growth regulators that can be incorporated seed coating compositions are described in US 2012/0108431, which is incorporated by reference in its entirety.

### Plant-Modifying Agents

The bacteria-derived lipid composition described herein include one or more heterologous plant-modifying agents. For example, the bacteria-derived lipid composition may encapsulate the heterologous plant-modifying agent. Alternatively or additionally, the heterologous plant-modifying agent can be embedded on or conjugated to the surface of the bacteria-derived lipid composition.

In some instances, the plant-modifying agent can include a peptide or a nucleic acid. The plant-modifying agent may be an agent that increases the fitness of a variety of plants or can be one that targets one or more specific plants (e.g., a specific species or genera of plants). Additionally, in some instances, the bacteria-derived lipid composition includes two or more (e.g., 2, 3, 4, 5, 6, 7, 8, 9, 10, or more than 10) different plant-modifying agents.

Further, in some instances, the heterologous plant-modifying agent (e.g., an agent including a nucleic acid molecule or peptide) can be modified. For example, the modification can be a chemical modification, e.g., conjugation to a marker, e.g., fluorescent marker or a radioactive marker. In other examples, the modification can include conjugation or operational linkage to a moiety that enhances the stability, delivery, targeting, bioavailability, or half-life of the agent, e.g., a lipid, a glycan, a polymer (e.g., PEG), a cation moiety.

### Polypeptides

The bacteria-derived lipid composition may include a polypeptide. In some instances, the bacteria-derived lipid composition includes a polypeptide or functional fragments or derivative thereof.

Examples of polypeptides include an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone.

Polypeptides may include naturally occurring polypeptides or recombinantly produced variants. In some instances, the polypeptide may be a functional fragments or variants thereof (e.g., an enzymatically active fragment or variant thereof). For example, the polypeptide may be a functionally active variant of any of the polypeptides described herein with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a polypeptide described herein or a naturally occurring polypeptide. In some instances, the polypeptide may have at least 50% (e.g., at least 50%, 60%, 70%, 80%, 90%, 95%, 97%, 99%, or greater) identity to a protein of interest.

The polypeptides may be formulated in a bacteria-derived lipid composition. The compositions disclosed herein may include any number or type (e.g., classes) of polypeptides, such as at least about any one of 1 polypeptide, 2, 3, 4, 5, 10, 15, 20, or more polypeptides. A suitable concentration of each polypeptide in the composition depends on factors such as efficacy, stability of the polypeptide, number of distinct polypeptides in the composition, the formulation, and methods of application of the composition. In some instances, each polypeptide in a liquid composition is from about 0.1 ng/mL to about 100 mg/mL. In some instances, each polypeptide in a solid composition is from about 0.1 ng/g to about 100 mg/g.

Methods of making a polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Methods for producing a polypeptide involve expression in plant cells, although recombinant proteins can also be produced using insect cells, yeast, bacteria, mammalian cells, or other cells under the control of appropriate promoters. Mammalian expression vectors may comprise nontranscribed elements such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture a recombinant polypeptide agent. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologics Manufacturing (Advances in Biochemical Engineering/Biotechnology), Springer (2014). Purification of proteins is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

In some instances, the bacteria-derived lipid composition includes an antibody or antigen binding fragment thereof. For example, an agent described herein may be an antibody that blocks or potentiates activity and/or function of a component. The antibody may act as an antagonist or agonist of a polypeptide (e.g., enzyme or cell receptor). The making and use of antibodies against a target antigen is known in the art. See, for example, Zhiqiang An (Ed.), Therapeutic Monoclonal Antibodies: From Bench to Clinic, 1st Edition, Wiley, 2009 and also Greenfield (Ed.), Antibodies: A Laboratory Manual, 2nd Edition, Cold Spring Harbor Laboratory Press, 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

### Nucleic Acids

In some instances, the bacteria-derived lipid composition includes a nucleic acid (a polynucleotide). Numerous nucleic acids are useful in the bacteria-derived lipid composition and methods described herein. The bacteria-derived lipid composition may include any number or type (e.g., classes) of heterologous nucleic acids (e.g., DNA molecule (e.g., plasmid) or RNA molecule, e.g., mRNA, guide RNA (gRNA), or inhibitory RNA molecule or precursor thereof (e.g., siRNA, shRNA, or miRNA or a precursor of any of these), or a hybrid DNA-RNA molecule), such as at least about 1 class or variant of a nucleic acid, or 2, 3, 4, 5, 10, 15, 20, or more classes or variants of nucleic acids. A suitable concentration of each nucleic acid in the composition depends on factors such as efficacy, stability of the nucleic acid, number of distinct nucleic acids, the formulation, and methods of application of the composition. Examples of nucleic acids useful herein include a DNA molecule (e.g., a plasmid), an mRNA, an siRNA, a Dicer substrate small interfering RNA (dsiRNA), an antisense RNA, a short interfering RNA (siRNA) or siRNA precursor (e.g., one or more strands of RNA that hybridize inter- or intra-molecularly to form at least partially double-stranded RNA having at least about 20 contiguous base-pairs), a short hairpin (shRNA), a microRNA (miRNA) or miRNA precursor, an asymmetric interfering RNA (aiRNA), a peptide nucleic acid (PNA), a morpholino, a locked nucleic acid (LNA), a piwi-interacting RNA (piRNA), a ribozyme, a deoxyribozymes (DNAzyme), an aptamer (DNA, RNA), a circular RNA (circRNA), a guide RNA (gRNA), or a DNA molecule encoding any of these RNAs.

### Nucleic Acids Encoding Peptides

In some instances, the bacteria-derived lipid composition includes a nucleic acid encoding a polypeptide. Nucleic acids encoding a polypeptide may have a length from about 10 to about 50,000 nucleotides (nts), about 25 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, about 5000 to about 6000 nts, about 6000 to about 7000 nts, about 7000 to about 8000 nts, about 8000 to about 9000 nts, about 9000 to about 10,000 nts, about 10,000 to about 15,000 nts, about 10,000 to about 20,000 nts, about 10,000 to about 25,000 nts, about 10,000 to about 30,000 nts, about 10,000 to about 40,000 nts, about 10,000 to about 45,000 nts, about 10,000 to about 50,000 nts, or any range there between.

The bacteria-derived lipid composition may also include active variants of a nucleic acid sequence of interest. In some instances, the variant of the nucleic acids has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire sequence, to a sequence of a nucleic acid of interest. In some instances, the bacteria-derived lipid composition includes an active polypeptide encoded by a nucleic acid variant. In some instances, the active polypeptide encoded by the nucleic acid variant has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, e.g., over a specified region or over the entire amino acid sequence, to a sequence of a polypeptide of interest or the naturally derived polypeptide sequence.

Certain methods for expressing a nucleic acid encoding a protein may involve expression in cells, including insect, yeast, plant, bacteria, or other cells under the control of appropriate promoters. Expression vectors may include nontranscribed elements, such as an origin of replication, a suitable promoter and enhancer, and other 5′ or 3′ flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green et al., Molecular Cloning: A Laboratory Manual, Fourth Edition, Cold Spring Harbor Laboratory Press, 2012.

Genetic modification using recombinant methods is generally known in the art. A nucleic acid sequence coding for a desired gene can be obtained using recombinant methods known in the art, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. Expression vectors can be suitable for replication and expression in bacteria. Expression vectors can also be suitable for replication and integration in eukaryotes. Typical cloning vectors contain transcription and translation terminators, initiation sequences, and promoters useful for expression of the desired nucleic acid sequence.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Alternatively, the promoter may be an inducible promoter. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transformed cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., FEBS Letters 479:79-82, 2000). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some instances, an organism may be genetically modified to alter expression of one or more proteins. Expression of the one or more proteins may be modified for a specific time, e.g., development or differentiation state of the organism. In one instances, the invention includes a composition to alter expression of one or more proteins, e.g., proteins that affect activity, structure, or function. Expression of the one or more proteins may be restricted to a specific location(s) or widespread throughout the organism.

mRNA

The bacteria-derived lipid composition may include a mRNA molecule, e.g., a mRNA molecule encoding a polypeptide. The mRNA molecule can be synthetic and modified, e.g., chemically. The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some instances, the modified RNA agent of interest described herein has modified nucleosides or nucleotides. Such modifications are known and are described, e.g., in WO 2012/019168. Additional modifications are described, e.g., in WO 2015/038892; WO 2015/038892; WO 2015/089511; WO 2015/196130; WO 2015/196118 and WO 2015/196128 A2, which are herein incorporated by reference in their entirety.

In some instances, the modified RNA encoding a polypeptide of interest has one or more terminal modification, e.g., a 5′ cap structure and/or a poly-A tail (e.g., of between 100-200 nucleotides in length). The 5′ cap structure may be selected from the group consisting of CapO, Capl, ARCA, inosine, NI-methyl-guanosine, 2′ fluoro-guanosine, 7-deaza-guanosine, 8-oxo-guanosine, 2-amino-guanosine, LNA-guanosine, and 2-azido-guanosine. In some cases, the modified RNAs also contain a 5′ UTR including at least one Kozak sequence, and a 3′ UTR. Such modifications are known and are described, e.g., in WO 2012/135805 and WO 2013/052523, which are incorporated herein by reference in their entirety. Additional terminal modifications are described, e.g., in WO 2014/164253 and WO 2016/011306, WO 2012/045075, and WO 2014/093924, which are incorporated herein by reference in their entirety. Chimeric enzymes for synthesizing capped RNA molecules (e.g., modified mRNA) which may include at least one chemical modification are described in WO 2014/028429, which is incorporated herein by reference in its entirety.

In some instances, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO 2013/151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO 2013/151666, WO 2013/151668, WO 2013/151663, WO 2013/151669, WO 2013/151670, WO 2013/151664, WO 2013/151665, WO 2013/151671, WO 2013/151672, WO 2013/151667 and WO 2013/151736. Methods of purification include purifying an RNA transcript including a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO 2014/152031); using ion (e.g., anion) exchange chromatography that allows for separation of longer RNAs up to 10,000 nucleotides in length via a scalable method (WO 2014/144767); and subjecting a modified mRNA sample to DNAse treatment (WO 2014/152030).

Formulations of modified RNAs are known and are described, e.g., in WO 2013/090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO 2013/151666, WO 2013/151668, WO 2013/151663, WO 2013/151669, WO 2013/151670, WO 2013/151664, WO 2013/151665, WO 2013/151736; Tables 6 and 7 International Publication No. WO 2013/151672; Tables 6, 178 and 179 of International Publication No. WO 2013/151671; Tables 6, 185 and 186 of International Publication No WO 2013/151667. Any of the foregoing may be synthesized as an IVT polynucleotide, chimeric polynucleotide or a circular polynucleotide, and each may include one or more modified nucleotides or terminal modifications.

### Inhibitory RNA

In some instances, the bacteria-derived lipid composition includes an inhibitory RNA molecule, e.g., that acts via the RNA interference (RNAi) pathway. In some instances, the inhibitory RNA molecule decreases the level of gene expression and/or decreases the level of a protein. In some instances, the inhibitory RNA molecule inhibits expression of a gene. For example, an inhibitory RNA molecule may include a short interfering RNA or its precursor, short hairpin RNA, and/or a microRNA or its precursor that targets a gene. Certain RNA molecules can inhibit gene expression through the biological process of RNA interference (RNAi). RNAi molecules include RNA or RNA-like structures typically containing 15-50 base pairs (such as about 18-25 base pairs) and having a nucleobase sequence identical (or complementary) or nearly identical (or substantially complementary) to a coding sequence in an expressed target gene within the cell. RNAi molecules include, but are not limited to: short interfering RNAs (siRNAs), double-strand RNAs (dsRNA), short hairpin RNAs (shRNA), meroduplexes, dicer substrates, and multivalent RNA interference (U.S. Pat. Nos. 8,084,599 8,349,809, 8,513,207 and 9,200,276). A shRNA is a RNA molecule including a hairpin turn that decreases expression of target genes via RNAi. shRNAs can be delivered to cells in the form of plasmids, e.g., viral or bacterial vectors, e.g., by transfection, electroporation, or transduction). A microRNA is a non-coding RNA molecule that typically has a length of about 21 or 22 nucleotides. MiRNAs bind to target sites on mRNA molecules and silence the mRNA, e.g., by causing cleavage of the mRNA, destabilization of the mRNA, or inhibition of translation of the mRNA. In some instances, the inhibitory RNA molecule decreases the level and/or activity of a negative regulator of function. In other instances, the inhibitor RNA molecule decreases the level and/or activity of an inhibitor of a positive regulator of function. The inhibitory RNA molecule can be chemically synthesized or transcribed in vitro.

In some instances, the nucleic acid is a DNA, a RNA, or a PNA. In some instances, the RNA is an inhibitory RNA. In some instances, the inhibitory RNA inhibits gene expression. In some instances, the nucleic acid is an mRNA, a modified mRNA, or a DNA molecule that increases expression of an enzyme (e.g., a metabolic recombinase, a helicase, an integrase, a RNAse, a DNAse, or an ubiquitination protein), a pore-forming protein, a signaling ligand, a cell penetrating peptide, a transcription factor, a receptor, an antibody, a nanobody, a gene editing protein (e.g., CRISPR-Cas system, TALEN, or zinc finger), riboprotein, a protein aptamer, or a chaperone. In some instances, the nucleic acid is an mRNA, a modified mRNA, or a DNA molecule that increases the expression of an enzyme (e.g., a metabolic enzyme, a recombinase enzyme, a helicase enzyme, an integrase enzyme, a RNAse enzyme, a DNAse enzyme, or an ubiquitination protein), a pore-forming protein, a signaling ligand, a cell penetrating peptide, a transcription factor, a receptor, an antibody, a nanobody, a gene editing protein (e.g., a CRISPR-Cas system, a TALEN, or a zinc finger), a riboprotein, a protein aptamer, or a chaperone. In some aspects, the nucleic acid encodes the enzyme, pore-forming protein, signaling ligand, cell penetrating peptide, transcription factor, receptor, antibody, nanobody, gene editing protein, riboprotein, protein aptamer, or chaperone. In some instances, the increase in expression is an increase in expression of about 5%, 10%, 15%, 20%, 30%, 40%, 50%, 60%, 70%, 80%, 90%, 100%, or more than 100% relative to a reference level (e.g., the expression in an untreated subject). In some instances, the increase in expression is an increase in expression of about 2× fold, about 4× fold, about 5× fold, about 10× fold, about 20× fold, about 25× fold, about 50× fold, about 75× fold, or about 100× fold or more, relative to a reference level (e.g., the expression in an untreated subject).

In some instances, the nucleic acid is an antisense RNA, a dsiRNA, a siRNA, a shRNA, a miRNA, an aiRNA, a PNA, a morpholino, a LNA, a piRNA, a ribozyme, a DNAzyme, an aptamer (DNA, RNA), a circRNA, a gRNA, or a DNA molecules (e.g., a plasmid) that acts to reduce expression of, e.g., an enzyme (a metabolic enzyme, a recombinase enzyme, a helicase enzyme, an integrase enzyme, a RNAse enzyme, a DNAse enzyme, a polymerase enzyme, a ubiquitination protein, a superoxide management enzyme, or an energy production enzyme), a transcription factor, a secretory protein, a structural factor (actin, kinesin, or tubulin), a riboprotein, a protein aptamer, a chaperone, a receptor, a signaling ligand, or a transporter. In some instances, the decrease in expression is a decrease in expression of about 5%, 10%, 15%, 20%, 30%, 40%, 50%, 60%, 70%, 80%, 90%, 100%, or more than 100% relative to a reference level (e.g., the expression in an untreated subject). In some instances, the decrease in expression is a decrease in expression of about 2× fold, about 4× fold, about 5× fold, about 10× fold, about 20× fold, about 25× fold, about 50× fold, about 75× fold, or about 100× fold or more, relative to a reference level (e.g., the expression in an untreated subject).

RNAi molecules include a sequence substantially complementary, or fully complementary, to all or a fragment of a target gene. RNAi molecules may complement sequences at the boundary between introns and exons to prevent the maturation of newly generated nuclear RNA transcripts of specific genes into mRNA for transcription. RNAi molecules complementary to specific genes can hybridize with the mRNA for a target gene and prevent its translation. The antisense molecule can be DNA, RNA, or a derivative or hybrid thereof. Examples of such derivative molecules include, but are not limited to, peptide nucleic acid (PNA) and phosphorothioate-based molecules such as deoxyribonucleic guanidine (DNG) or ribonucleic guanidine (RNG).

RNAi molecules can be provided as ready-to-use RNA synthesized in vitro or as sense and antisense RNA sequences (or DNA encoding sense and antisense RNA sequences) transfected into cells which will yield RNAi molecules upon transcription. Hybridization of the RNA molecule with, e.g., the target mRNA results in degradation of the hybridized complex by RNAse H and/or inhibition of the formation of translation complexes. Both result in a failure to produce the product of the original gene.

The length of the RNAi molecule that hybridizes to the transcript of interest may be around 10 nucleotides, between about 15 or 30 nucleotides, or about 15, 16, 17, 18, 19, 20, 21, 22, 23, 24, 25, 26, 27, 28, 29, 30 or more nucleotides. In embodiments, the RNAi molecule hybridizes to the transcript of interest to form a perfectly or near-perfectly double-stranded region of at least about 17 base pairs; in embodiments the double-stranded region includes at least about 10 contiguous base pairs. The degree of identity of the antisense sequence to the targeted transcript may be at least 75%, at least 80%, at least 85%, at least 90%, or at least 95.

RNAi molecules may also include overhangs, i.e., typically unpaired, overhanging nucleotides which are not directly involved in the double helical structure normally formed by the core sequences of the herein defined pair of sense strand and antisense strand. RNAi molecules may contain 3′ and/or 5′ overhangs of about 1-5 bases independently on each of the sense strands and antisense strands. In some instances, both the sense strand and the antisense strand contain 3′ and 5′ overhangs. In some instances, one or more of the 3′ overhang nucleotides of one-strand base pairs with one or more 5′ overhang nucleotides of the other strand. In other instances, the one or more of the 3′ overhang nucleotides of one strand base do not pair with the one or more 5′ overhang nucleotides of the other strand. The sense and antisense strands of an RNAi molecule may or may not contain the same number of nucleotide bases. The antisense and sense strands may form a duplex wherein the 5′ end only has a blunt end, the 3′ end only has a blunt end, both the 5′ and 3′ ends are blunt ended, or neither the 5′ end nor the 3′ end are blunt ended. In another instance, one or more of the nucleotides in the overhang contains a thiophosphate, phosphorothioate, deoxynucleotide inverted (3′ to 3′ linked) nucleotide or is a modified ribonucleotide or deoxynucleotide.

Small interfering RNA (siRNA) molecules include a nucleotide sequence that is identical to about 15 to about 25 contiguous nucleotides of the target mRNA. In some instances, the siRNA sequence commences with the dinucleotide AA, includes a GC-content of about 30-70% (about 30-60%, about 40-60%, or about 45%-55%), and does not have a high percentage identity to any nucleotide sequence other than the target in the genome in which it is to be introduced, for example as determined by standard BLAST search.

siRNAs and shRNAs resemble intermediates in the processing pathway of the endogenous microRNA (miRNA) genes (Bartel, Cell 116:281-297, 2004). In some instances, siRNAs can function as miRNAs and vice versa (Zeng et al., Mol. Cell 9:1327-1333, 2002; Doench et al., Genes Dev. 17:438-442, 2003). Exogenous siRNAs downregulate mRNAs with seed complementarity to the siRNA (Birmingham et al., Nat. Methods 3:199-204, 2006). Multiple target sites within a 3′ UTR give stronger downregulation (Doench et al., Genes Dev. 17:438-442, 2003).

Known effective siRNA sequences and cognate binding sites are also well represented in the relevant literature. RNAi molecules are readily designed and produced by technologies known in the art. In addition, there are computational tools that increase the chance of finding effective and specific sequence motifs (Pei et al., Nat. Methods 3(9):670-676, 2006; Reynolds et al., Nat. Biotechnol. 22(3):326-330, 2004; Khvorova et al., Nat. Struct. Biol. 10(9):708-712, 2003; Schwarz et al., Cell 115(2):199-208, 2003; Ui-Tei et al., Nucleic Acids Res. 32(3):936-948, 2004; Heale et al., Nucleic Acids Res. 33(3):e30, 2005; Chalk et al., Biochem. Biophys. Res. Commun. 319(1):264-274, 2004; and Amarzguioui et al., Biochem. Biophys. Res. Commun. 316(4):1050-1058, 2004).

The RNAi molecule modulates expression of RNA encoded by a gene. Because multiple genes can share some degree of sequence homology with each other, in some instances, the RNAi molecule can be designed to target a class of genes with sufficient sequence homology. In some instances, the RNAi molecule can contain a sequence that has complementarity to sequences that are shared amongst different gene targets or are unique for a specific gene target. In some instances, the RNAi molecule can be designed to target conserved regions of an RNA sequence having homology between several genes thereby targeting several genes in a gene family (e.g., different gene isoforms, splice variants, mutant genes, etc.). In some instances, the RNAi molecule can be designed to target a sequence that is unique to a specific RNA sequence of a single gene.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that such modifications can increase nuclease resistance and/or serum stability, or decrease immunogenicity.

In some instances, the RNAi molecule or its precursor is linked to a delivery polymer via a physiologically labile bond or linker. The physiologically labile linker is selected such that it undergoes a chemical transformation (e.g., cleavage) when present in certain physiological conditions, (e.g., disulfide bond cleaved in the reducing environment of the cell cytoplasm). Release of the molecule from the polymer, by cleavage of the physiologically labile linkage, facilitates interaction of the molecule with the appropriate cellular components for activity.

The RNAi molecule-polymer conjugate may be formed by covalently linking the molecule to the polymer. The polymer is polymerized or modified such that it contains a reactive group A. The RNAi molecule is also polymerized or modified such that it contains a reactive group B. Reactive groups A and B are chosen such that they can be linked via a reversible covalent linkage using methods known in the art.

Conjugation of the RNAi molecule to the polymer can be performed in the presence of an excess of polymer. Because the RNAi molecule and the polymer may be of opposite charge during conjugation, the presence of excess polymer can reduce or eliminate aggregation of the conjugate. Alternatively, an excess of a carrier polymer, such as a polycation, can be used. The excess polymer can be removed from the conjugated polymer prior to administration of the conjugate. Alternatively, the excess polymer can be co-administered with the conjugate.

The making and use of inhibitory agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press (2010).

### Gene Editing

The bacteria-derived lipid composition may include a component of a gene editing system. For example, the agent may introduce an alteration (e.g., insertion, deletion (e.g., knockout), translocation, inversion, single point mutation, or other mutation) in a gene. Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al., Trends Biotechnol. 31(7):397-405, 2013.

Additional descriptions about the component and process of the gene editing system may be found in International Patent Application Publication No. WO 2021/041301, which is incorporated herein by reference in its entirety.

### Heterologous Therapeutic Agents

The bacteria-derived lipid composition can include a therapeutic agent (e.g., an agent that affects an animal (e.g., a mammal, e.g., a human), an animal pathogen, or a pathogen vector thereof, and can be loaded into a bacteria-derived lipid composition), such as a therapeutic peptide, a therapeutic nucleic acid (e.g., a therapeutic RNA), a therapeutic small molecule, or a pathogen control agent (e.g., antifungal agent, an antibacterial agent, a virucidal agent, an anti-viral agent, an insecticidal agent, a nematicidal agent, an antiparasitic agent, or an insect repellent). The bacteria-derived lipid composition loaded with such agents can be formulated with a pharmaceutically acceptable carrier for delivery to an animal, an animal pathogen, or a pathogen vector thereof.

### Antibacterial Agents

The bacteria-derived lipid composition can further include an antibacterial agent. For example, a bacteria-derived lipid composition including an antibiotic can be administered to an animal in an amount and for a time sufficient to: reach a target level (e.g., a predetermined or threshold level) of antibiotic concentration inside or on the animal; and/or treat or prevent a bacterial infection in the animal. The antibacterials may be formulated in a bacteria-derived lipid composition for any of the methods described herein, and in certain instances, may be associated with the bacteria-derived lipid composition. In some instances, the bacteria-derived lipid composition includes two or more (e.g., 2, 3, 4, 5, 6, 7, 8, 9, 10, or more than 10) different antibacterial agents.

As used herein, the term “antibacterial agent” refers to a material that kills or inhibits the growth, proliferation, division, reproduction, or spread of bacteria, such as phytopathogenic bacteria, and includes bactericidal (e.g., disinfectant compounds, antiseptic compounds, or antibiotics) or bacteriostatic agents (e.g., compounds or antibiotics). Bactericidal antibiotics kill bacteria, while bacteriostatic antibiotics only slow their growth or reproduction.

Exemplary bactericides include those already discussed above relating to Antibacterial agents in the section of “Heterologous agricultural agents.”

As antiseptics (i.e., germicide agents that can be used on human or animal body, skin, mucoses, wounds and the like), few of the above mentioned disinfectants can be used, under proper conditions (mainly concentration, pH, temperature and toxicity toward man/animal). Among them, important are: properly diluted chlorine preparations (i.e., Daquin's solution, 0.5% sodium or potassium hypochlorite solution, pH-adjusted to pH 7-8, or 0.5-1% solution of sodium benzenesulfochloramide (chloramine B)), some iodine preparations, such as iodopovidone in various galenics (ointment, solutions, wound plasters), in the past also Lugol's solution, peroxides as urea perhydrate solutions and pH-buffered 0.1-0.25% peracetic acid solutions, alcohols with or without antiseptic additives, used mainly for skin antisepsis, weak organic acids such as sorbic acid, benzoic acid, lactic acid and salicylic acid some phenolic compounds, such as hexachlorophene, triclosan and Dibromol, and cation-active compounds, such as 0.05-0.5% benzalkonium, 0.5-4% chlorhexidine, 0.1-2% octenidine solutions.

The bacteria-derived lipid composition may include an antibiotic. Any antibiotic known in the art may be used. Antibiotics are commonly classified based on their mechanism of action, chemical structure, or spectrum of activity.

The antibiotic described herein may target any bacterial function or growth processes and may be either bacteriostatic (e.g., slow or prevent bacterial growth) or bactericidal (e.g., kill bacteria). In some instances, the antibiotic is a bactericidal antibiotic. In some instances, the bactericidal antibiotic is one that targets the bacterial cell wall (e.g., penicillins and cephalosporins); one that targets the cell membrane (e.g., polymyxins); or one that inhibits essential bacterial enzymes (e.g., rifamycins, lipiarmycins, quinolones, and sulfonamides). In some instances, the bactericidal antibiotic is an aminoglycoside (e.g., kasugamycin). In some instances, the antibiotic is a bacteriostatic antibiotic. In some instances the bacteriostatic antibiotic targets protein synthesis (e.g., macrolides, lincosamides, and tetracyclines). Additional classes of antibiotics that may be used herein include cyclic lipopeptides (such as daptomycin), glycylcyclines (such as tigecycline), oxazolidinones (such as linezolid), or lipiarmycins (such as fidaxomicin). Examples of antibiotics include rifampicin, ciprofloxacin, doxycycline, ampicillin, and polymyxin B. The antibiotic described herein may have any level of target specificity (e.g., narrow- or broad-spectrum). In some instances, the antibiotic is a narrow-spectrum antibiotic, and thus targets specific types of bacteria, such as gram-negative or gram-positive bacteria. Alternatively, the antibiotic may be a broad-spectrum antibiotic that targets a wide range of bacteria.

Examples of antibacterial agents suitable for the treatment of animals include Penicillins (Amoxicillin, Ampicillin, Bacampicillin, Carbenicillin, Cloxacillin, Dicloxacillin, Flucloxacillin, Mezlocillin, Nafcillin, Oxacillin, Penicillin G, Crysticillin 300 A.S., Pentids, Permapen, Pfizerpen, Pfizerpen-AS, Wycillin, Penicillin V, Piperacillin, Pivampicillin, Pivmecillinam, Ticarcillin), Cephalosporins (Cefacetrile (cephacetrile), Cefadroxil (cefadroxyl), Cefalexin (cephalexin), Cefaloglycin (cephaloglycin), Cefalonium (cephalonium), Cefaloridine (cephaloradine), Cefalotin (cephalothin), Cefapirin (cephapirin), Cefatrizine, Cefazaflur, Cefazedone, Cefazolin (cephazolin), Cefradine (cephradine), Cefroxadine, Ceftezole, Cefaclor, Cefamandole, Cefmetazole, Cefonicid, Cefotetan, Cefoxitin, Cefprozil (cefproxil), Cefuroxime, Cefuzonam, Cefcapene, Cefdaloxime, Cefdinir, Cefditoren, Cefetamet, Cefixime, Cefmenoxime, Cefodizime, Cefotaxime, Cefpimizole, Cefpodoxime, Cefteram, Ceftibuten, Ceftiofur, Ceftiolene, Ceftizoxime, Ceftriaxone, Cefoperazone, Ceftazidime, Cefclidine, Cefepime, Cefluprenam, Cefoselis, Cefozopran, Cefpirome, Cefquinome, Ceftobiprole, Ceftaroline, Cefaclomezine, Cefaloram, Cefaparole, Cefcanel, Cefedrolor, Cefempidone, Cefetrizole, Cefivitril, Cefmatilen, Cefmepidium, Cefovecin, Cefoxazole, Cefrotil, Cefsumide, Cefuracetime, Ceftioxide, Combinations, Ceftazidime/Avibactam, Ceftolozane/Tazobactam), Monobactams (Aztreonam), Carbapenems (Imipenem, Imipenem/cilastatin, Doripenem, Ertapenem, Meropenem, Meropenem/vaborbactam), Macrolide (Azithromycin, Erythromycin, Clarithromycin, Dirithromycin, Roxithromycin, Telithromycin), Lincosamides (Clindamycin, Lincomycin), Streptogramins (Pristinamycin, Quinupristin/dalfopristin), Aminoglycoside (Amikacin, Gentamicin, Kanamycin, Neomycin, Netilmicin, Paromomycin, Streptomycin, Tobramycin), Quinolone (Flumequine, Nalidixic acid, Oxolinic acid, Piromidic acid, Pipemidic acid, Rosoxacin, Second Generation, Ciprofloxacin, Enoxacin, Lomefloxacin, Nadifloxacin, Norfloxacin, Ofloxacin, Pefloxacin, Rufloxacin, Balofloxacin, Gatifloxacin, Grepafloxacin, Levofloxacin, Moxifloxacin, Pazufloxacin, Sparfloxacin, Temafloxacin, Tosufloxacin, Besifloxacin, Delafloxacin, Clinafloxacin, Gemifloxacin, Prulifloxacin, Sitafloxacin, Trovafloxacin), Sulfonamides (Sulfamethizole, Sulfamethoxazole, Sulfisoxazole, Trimethoprim-Sulfamethoxazole), Tetracycline (Demeclocycline, Doxycycline, Minocycline, Oxytetracycline, Tetracycline, Tigecycline), Other (Lipopeptides, Fluoroquinolone, Lipoglycopeptides, Cephalosporin, Macrocyclics, Chloramphenicol, Metronidazole, Tinidazole, Nitrofurantoin, Glycopeptides, Vancomycin, Teicoplanin, Lipoglycopeptides, Telavancin, Oxazolidinones, Linezolid, Cycloserine 2, Rifamycins, Rifampin, Rifabutin, Rifapentine, Rifalazil, Polypeptides, Bacitracin, Polymyxin B, Tuberactinomycins, Viomycin, Capreomycin).

One skilled in the art will appreciate that a suitable concentration of each antibiotic in the composition depends on factors such as efficacy, stability of the antibiotic, number of distinct antibiotics, the formulation, and methods of application of the composition.

### Antifungal Agents

The bacteria-derived lipid composition described herein can further include an antifungal agent. For example, a bacteria-derived lipid composition including an antifungal can be administered to an animal in an amount and for a time sufficient to reach a target level (e.g., a predetermined or threshold level) of antifungal concentration inside or on the animal; and/or treat or prevent a fungal infection in the animal. The antifungals described herein may be formulated in a bacteria-derived lipid composition for any of the methods described herein, and in certain instances, may be associated with the bacteria-derived lipid composition. In some instances, the bacteria-derived lipid composition includes two or more (e.g., 2, 3, 4, 5, 6, 7, 8, 9, 10, or more than 10) different antifungal agents.

As used herein, the term “fungicide” or “antifungal agent” refers to a substance that kills or inhibits the growth, proliferation, division, reproduction, or spread of fungi, such as fungi that are pathogenic to animals. Many different types of antifungal agent have been produced commercially. Non limiting examples of antifungal agents include: Allylamines (Amorolfin, Butenafine, Naftifine, Terbinafine), Imidazoles ((Bifonazole, Butoconazole, Clotrimazole, Econazole, Fenticonazole, Ketoconazole, Isoconazole, Luliconazole, Miconazole, Omoconazole, Oxiconazole, Sertaconazole, Sulconazole, Tioconazole, Terconazole); Triazoles (Albaconazole, Efinaconazole, Fluconazole, Isavuconazole, Itraconazole, Posaconazole, Ravuconazole, Terconazole, Voriconazole), Thiazoles (Abafungin), Polyenes (Amphotericin B, Nystatin, Natamycin, Trichomycin), Echinocandins (Anidulafungin, Caspofungin, Micafungin), Other (Tolnaftate, Flucytosine, Butenafine, Griseofulvin, Ciclopirox, Selenium sulfide, Tavaborole). One skilled in the art will appreciate that a suitable concentration of each antifungal in the composition depends on factors such as efficacy, stability of the antifungal, number of distinct antifungals, the formulation, and methods of application of the composition.

### Insecticides

The bacteria-derived lipid composition can further include an insecticide. For example, the insecticide can decrease the fitness of (e.g., decrease growth or kill) an insect vector of an animal pathogen. A bacteria-derived lipid composition including an insecticide can be contacted with an insect, in an amount and for a time sufficient to: (a) reach a target level (e.g., a predetermined or threshold level) of insecticide concentration inside or on the insect; and (b) decrease fitness of the insect. In some instances, the insecticide can decrease the fitness of (e.g., decrease growth or kill) a parasitic insect. A bacteria-derived lipid composition including an insecticide can be contacted with a parasitic insect, or an animal infected therewith, in an amount and for a time sufficient to: (a) reach a target level (e.g., a predetermined or threshold level) of insecticide concentration inside or on the parasitic insect; and (b) decrease the fitness of the parasitic insect. The insecticides described herein may be formulated in a bacteria-derived lipid composition for any of the methods described herein, and in certain instances, may be associated with the bacteria-derived lipid composition. In some instances, the bacteria-derived lipid composition includes two or more (e.g., 2, 3, 4, 5, 6, 7, 8, 9, 10, or more than 10) different insecticide agents.

As used herein, the term “insecticide” or “insecticidal agent” refers to a substance that kills or inhibits the growth, proliferation, reproduction, or spread of insects, such as insect vectors of animal pathogens or parasitic insects. Non limiting examples of insecticides are shown in Table 4 of WO 2021/041301, which is incorporated herein by reference in its entirety. Additional non-limiting examples of suitable insecticides include those already discussed above relating to Insecticides in the section of “Heterologous agricultural agents.” One skilled in the art will appreciate that a suitable concentration of each insecticide in the composition depends on factors such as efficacy, stability of the insecticide, number of distinct insecticides, the formulation, and methods of application of the composition.

### Nematicide

The bacteria-derived lipid composition can further include a nematicide as a therapeutic agent. Non limiting examples of nematicides used as a therapeutic agent include those already discussed above relating to Nematicide in the section of “Heterologous agricultural agents.”

### Antiparasitic Agent

The bacteria-derived lipid composition can further include an antiparasitic agent. For example, the antiparasitic can decrease the fitness of (e.g., decrease growth or kill) a parasitic protozoan. A bacteria-derived lipid composition including an antiparasitic as described herein can be contacted with a protozoan in an amount and for a time sufficient to: (a) reach a target level (e.g., a predetermined or threshold level) of antiparasitic concentration inside or on the protozoan, or animal infected therewith; and (b) decrease fitness of the protozoan. This can be useful in the treatment or prevention of parasites in animals. For example, a bacteria-derived lipid composition including an antiparasitic agent as described herein can be administered to an animal in an amount and for a time sufficient to: reach a target level (e.g., a predetermined or threshold level) of antiparasitic concentration inside or on the animal; and/or treat or prevent a parasite (e.g., parasitic nematode, parasitic insect, or protozoan) infection in the animal. The antiparasitic described herein may be formulated in a bacteria-derived lipid composition for any of the methods described herein, and in certain instances, may be associated with the bacteria-derived lipid composition thereof. In some instances, the bacteria-derived lipid composition includes two or more (e.g., 2, 3, 4, 5, 6, 7, 8, 9, 10, or more than 10) different antiparasitic agents.

As used herein, the term “antiparasitic” or “antiparasitic agent” refers to a substance that kills or inhibits the growth, proliferation, reproduction, or spread of parasites, such as parasitic protozoa, parasitic nematodes, or parasitic insects. Examples of antiparasitic agents include Antihelmintics (Bephenium, Diethylcarbamazine, Ivermectin, Niclosamide, Piperazine, Praziquantel, Pyrantel, Pyrvinium, Benzimidazoles, Albendazole, Flubendazole, Mebendazole, Thiabendazole, Levamisole, Nitazoxanide, Monopantel, Emodepside, Spiroindoles), Scabicides (Benzyl benzoate, Benzyl benzoate/disulfiram, Lindane, Malathion, Permethrin), Pediculicides (Piperonyl butoxide/pyrethrins, Spinosad, Moxidectin), Scabicides (Crotamiton), Anticestodes (Niclosamide, Pranziquantel, Albendazole), Antiamoebics (Rifampin, Apmphotericin B); or Antiprotozoals (Melarsoprol, Eflornithine, Metronidazole, Tinidazole, Miltefosine, Artemisinin). In certain instances, the antiparasitic agent may be use for treating or preventing infections in livestock animals, e.g., Levamisole, Fenbendazole, Oxfendazole, Albendazole, Moxidectin, Eprinomectin, Doramectin, Ivermectin, or Clorsulon. One skilled in the art will appreciate that a suitable concentration of each antiparasitic in the composition depends on factors such as efficacy, stability of the antiparasitic, number of distinct antiparasitics, the formulation, and methods of application of the composition.

### Antiviral Agent

The bacteria-derived lipid composition can further include an antiviral agent. A bacteria-derived lipid composition including an antiviral agent can be administered to an animal in an amount and for a time sufficient to reach a target level (e.g., a predetermined or threshold level) of antiviral concentration inside or on the animal; and/or to treat or prevent a viral infection in the animal. The antivirals described herein may be formulated in a bacteria-derived lipid composition for any of the methods described herein, and in certain instances, may be associated with the bacteria-derived lipid composition. In some instances, the bacteria-derived lipid composition includes two or more (e.g., 2, 3, 4, 5, 6, 7, 8, 9, 10, or more than 10) different antivirals.

As used herein, the term “antiviral” or “virucide” refers to a substance that kills or inhibits the growth, proliferation, reproduction, development, or spread of viruses, such as viral pathogens that infect animals. A number of agents can be employed as an antiviral, including chemicals or biological agents (e.g., nucleic acids, e.g., dsRNA). Examples of antiviral agents useful herein include Abacavir, Acyclovir (Aciclovir), Adefovir, Amantadine, Amprenavir (Agenerase), Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Fusion inhibitor, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Nucleoside analogues, Norvir, Oseltamivir (Tamiflu), Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), or Zidovudine. One skilled in the art will appreciate that a suitable concentration of each antiviral in the composition depends on factors such as efficacy, stability of the antivirals, number of distinct antivirals, the formulation, and methods of application of the composition.

### Repellents

The bacteria-derived lipid composition can further include a repellent. For example, the repellent can repel a vector of animal pathogens, such as insects. The repellent described herein may be formulated in a bacteria-derived lipid composition for any of the methods described herein, and in certain instances, may be associated with the bacteria-derived lipid composition. In some instances, the bacteria-derived lipid composition includes two or more (e.g., 2, 3, 4, 5, 6, 7, 8, 9, 10, or more than 10) different repellents.

For example, a bacteria-derived lipid composition including a repellent as described herein can be contacted with an insect vector or a habitat of the vector in an amount and for a time sufficient to: (a) reach a target level (e.g., a predetermined or threshold level) of repellent concentration; and/or (b) decrease the levels of the insect near or on nearby animals relative to a control. Alternatively, a bacteria-derived lipid composition including a repellent as described herein can be contacted with an animal in an amount and for a time sufficient to: (a) reach a target level (e.g., a predetermined or threshold level) of repellent concentration; and/or (b) decrease the levels of the insect near or on the animal relative to an untreated animal.

Some examples of well-known insect repellents include: benzil; benzyl benzoate; 2,3,4,5-bis(butyl-2-ene)tetrahydrofurfural (MGK Repellent 11); butoxypolypropylene glycol; N-butylacetanilide; normal-butyl-6,6-dimethyl-5,6-dihydro-1,4-pyrone-2-carboxylate (Indalone); dibutyl adipate; dibutyl phthalate; di-normal-butyl succinate (Tabatrex); N,N-diethyl-meta-toluamide (DEET); dimethyl carbate (endo,endo)-dimethyl bicyclo[2.2.1]hept-5-ene-2,3-dicarboxylate); dimethyl phthalate; 2-ethyl-2-butyl-1,3-propanediol; 2-ethyl-1,3-hexanediol (Rutgers 612); di-normal-propyl isocinchomeronate (MGK Repellent 326); 2-phenylcyclohexanol; p-methane-3,8-diol, and normal-propyl N,N-diethylsuccinamate. Other repellents include citronella oil, dimethyl phthalate, normal-butylmesityl oxide oxalate and 2-ethyl hexanediol-1,3 (See, Kirk-Othmer Encyclopedia of Chemical Technology, 2nd Ed., Vol. 11: 724-728; and The Condensed Chemical Dictionary, 8th Ed., p 756).

In some instances, the repellent is an insect repellent, including synthetic or nonsynthetic insect repellents. Examples of synthetic insect repellents include methyl anthranilate and other anthranilate-based insect repellents, benzaldehyde, DEET (N,N-diethyl-m-toluamide), dimethyl carbate, dimethyl phthalate, icaridin (i.e., picaridin, Bayrepel, and KBR 3023), indalone (e.g., as used in a “6-2-2” mixture (60% Dimethyl phthalate, 20% Indalone, 20% Ethylhexanediol), IR3535 (3-[N-Butyl-N-acetyl]-aminopropionic acid, ethyl ester), metofluthrin, permethrin, SS220, or tricyclodecenyl allyl ether. Examples of natural insect repellents include beautyberry (Callicarpa) leaves, birch tree bark, bog myrtle (Myrica gale), catnip oil (e.g., nepetalactone), citronella oil, essential oil of the lemon eucalyptus (Corymbia citriodora; e.g., p-menthane-3,8-diol (PMD)), neem oil, lemongrass, tea tree oil from the leaves of Melaleuca alternifolia, tobacco, or extracts thereof.

### Use of the Bacteria-Derived Lipid Composition

The bacteria-derived lipid compositions are useful in a variety of agricultural or therapeutic applications. Examples of methods of using the bacteria-derived lipid composition are described further below.

### Delivery to a Plant

Provided herein are methods of delivering a bacteria-derived lipid composition to a plant, e.g., by contacting the plant, or parts thereof (e.g., leaf, seed, pollen, root, fruit, shoot, flower, cells, protoplasts, or tissue (e.g., meristematic tissue) of the plant), with the bacteria-derived lipid composition. In some instances, plants may be treated with bacteria-derived lipid composition not including a heterologous functional agent. In other instances, the bacteria-derived lipid composition includes a heterologous functional agent, e.g., pesticidal agents (e.g., antibacterial agents, antifungal agents, nematicides, molluscicides, virucides, herbicides), pest control agents (e.g., repellents), fertilizing agents, or plant-modifying agents.

In some embodiments, provided herein is a method of increasing the fitness of a plant, the method including delivering a bacteria-derived lipid composition to a plant, e.g., by contacting the plant, or parts thereof (e.g., leaf, seed, pollen, root, fruit, shoot, flower, cells, protoplasts, or tissue (e.g., meristematic tissue) of the plant) with the bacteria-derived lipid composition described herein (e.g., in an effective amount and duration) to increase the fitness of the plant relative to an untreated plant (e.g., a plant that has not been delivered the bacteria-derived lipid composition.

An increase in the fitness of the plant as a consequence of delivery of a bacteria-derived lipid composition can manifest in a number of ways, e.g., thereby resulting in a better production of the plant, for example, an improved yield, improved vigor of the plant (e.g., improved tolerance of abiotic or biotic stress or improved resistance to pests) or improved quality of the harvested product from the plant. An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional agricultural agents.

An increase in the fitness of a plant as a consequence of delivery of a bacteria-derived lipid composition can also be measured by other methods, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional agricultural agents.

In the cases where an herbicide is included in the bacteria-derived lipid composition, provided herein is a method of decreasing the fitness of or kill weeds. In such instances, the method may be effective to decrease the fitness of the weed by about 2%, 5%, 10%, 20%, 30%, 40%, 50%, 60%, 70%, 80%, 90%, 100%, or more in comparison to an untreated weed (e.g., a weed to which the bacteria-derived lipid composition has not been administered). For example, the method may be effective to kill the weed, thereby decreasing a population of the weed by about 2%, 5%, 10%, 20%, 30%, 40%, 50%, 60%, 70%, 80%, 90%, 100%, or more in comparison to an untreated weed. In some instances, the method substantially eliminates the weed.

Plants that can be delivered a bacteria-derived lipid composition (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., meristematic tissue, vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

Examples of types of plants and weeds that can be treated in accordance with the a bacteria-derived lipid composition may be found in WO 2021/041301, which is incorporated herein by reference in its entirety.

### Delivery to a Plant Pest

Provided herein are methods of delivering a bacteria-derived lipid composition to a plant pest, e.g., by contacting the plant pest with the bacteria-derived lipid composition. In some instances, the plant pests may be treated with the bacteria-derived lipid composition not including a heterologous functional agent. In other instances, the bacteria-derived lipid composition includes a heterologous functional agent, e.g., pesticidal agents (e.g., antibacterial agents, antifungal agents, nematicides, molluscicides, virucides, or herbicides) or pest control agents (e.g., repellents). For example, the methods can be useful for decreasing the fitness of a pest, e.g., to prevent or treat a pest infestation as a consequence of delivery of a bacteria-derived lipid composition.

In some embodiments, provided herein is a method of decreasing the fitness of a pest, the method including delivering to the pest the bacteria-derived lipid composition described herein (e.g., in an effective amount and for an effective duration) to decrease the fitness of the pest relative to an untreated pest (e.g., a pest that has not been delivered the bacteria-derived lipid composition).

In some embodiments, provided herein is a method of decreasing a fungal infection in (e.g., treating) a plant having a fungal infection, wherein the method includes delivering to the plant pest the bacteria-derived lipid composition described herein. In some embodiments, provided herein is a method of decreasing a fungal infection in (e.g., treating) a plant having a fungal infection, wherein the method includes delivering to the plant pest the bacteria-derived lipid composition described herein, and wherein the bacteria-derived lipid composition includes an antifungal agent.

In some embodiments, provided herein is a method of decreasing a bacterial infection in (e.g., treating) a plant having a bacterial infection, wherein the method includes delivering to the plant pest pest the bacteria-derived lipid composition described herein.

In some embodiments, provided herein is a method of decreasing a bacterial infection in (e.g., treating) a plant having a bacterial infection, wherein the method includes delivering to the plant pest the bacteria-derived lipid composition described herein, and wherein the bacteria-derived lipid composition includes an antibacterial agent.

In some embodiments, provided herein is a method of decreasing the fitness of an insect plant pest, wherein the method includes delivering to the insect plant pest the bacteria-derived lipid composition described herein.

In some embodiments, provided herein is a method of decreasing the fitness of an insect plant pest, wherein the method includes delivering to the insect plant pest the bacteria-derived lipid composition described herein, and wherein the bacteria-derived lipid composition includes an insecticidal agent.

In some embodiments, provided herein is a method of decreasing the fitness of a nematode plant pest, wherein the method includes delivering to the nematode plant pest the bacteria-derived lipid composition described herein.

In some embodiments, provided herein is a method of decreasing the fitness of a nematode plant pest, wherein the method includes delivering to the nematode plant pest the bacteria-derived lipid composition described herein, and wherein the bacteria-derived lipid composition includes a nematicidal agent.

In some embodiments, provided herein is a method of decreasing the fitness of a weed, wherein the method includes delivering to the weed the bacteria-derived lipid composition described herein.

In some embodiments, provided herein is a method of decreasing the fitness of a weed, wherein the method includes delivering to the weed the bacteria-derived lipid composition described herein, and wherein the bacteria-derived lipid composition includes an herbicidal agent (e.g. Glufosinate).

Suitable antibacterial agents, insecticidal agents, nematicidal agents, herbicidal agents include those already described above.

A decrease in the fitness of the pest as a consequence of delivery of the bacteria-derived lipid composition can manifest in a number of ways. In some instances, the decrease in fitness of the pest may manifest as a deterioration or decline in the physiology of the pest (e.g., reduced health or survival) as a consequence of delivery of the bacteria-derived lipid composition. In some instances, the fitness of an organism may be measured by one or more parameters, including, but not limited to, reproductive rate, fertility, lifespan, viability, mobility, fecundity, pest development, body weight, metabolic rate or activity, or survival in comparison to a pest to which the bacteria-derived lipid composition has not been administered.

In some instances, the decrease in pest fitness may manifest as a decrease in the production of one or more nutrients in the pest (e.g., vitamins, carbohydrates, amino acids, or polypeptides) in comparison to a pest to which the bacteria-derived lipid composition has not been administered.

In some instances, the decrease in pest fitness may manifest as an increase in the pest's sensitivity to a pesticidal agent or an allelochemical agent, and/or a decrease in the pest's resistance to a pesticidal agent in comparison to a pest to which the bacteria-derived lipid composition has not been administered.

In some instances, the methods or compositions provided herein may be effective to decease the pest's resistance to parasites or pathogens (e.g., fungal, bacterial, or viral pathogens or parasites) in comparison to a pest to which the bacteria-derived lipid composition has not been administered.

In some instances, the methods or compositions provided herein may be effective to decrease the pest's ability to carry or transmit a plant pathogen (e.g., plant virus (e.g., TYLCV) or a plant bacterium (e.g., Agrobacterium spp.)) in comparison to a pest to which the bacteria-derived lipid composition has not been administered.

### Delivery to a Plant Symbiont

Provided herein are methods of delivering to a plant symbiont the bacteria-derived lipid composition disclosed herein. Included are methods for delivering the bacteria-derived lipid composition to a symbiont (e.g., a bacterial endosymbiont, a fungal endosymbiont, or an insect) by contacting the symbiont with the bacteria-derived lipid composition. The methods can be useful for increasing the fitness of plant symbiont, e.g., a symbiont that is beneficial to the fitness of a plant. In some instances, plant symbionts may be treated with the bacteria-derived lipid composition not including a heterologous functional agent. In other instances, the a bacteria-derived lipid composition includes a heterologous functional agent, e.g., fertilizing agents.

As such, the methods can be used to increase the fitness of a plant symbiont. In one aspect, provided herein is a method of increasing the fitness of a symbiont, the method including delivering to the symbiont the a bacteria-derived lipid composition described herein (e.g., in an effective amount and for an effective duration) to increase the fitness of the symbiont relative to an untreated symbiont (e.g., a symbiont that has not been delivered the a bacteria-derived lipid composition).

In some embodiments, provided herein is a method of increasing the fitness of a fungus (e.g., a fungal endosymbiont of a plant), wherein the method includes delivering to the endosymbiont the bacteria-derived lipid composition described herein.

In some embodiments, provided herein is a method of increasing the fitness of a bacterium (e.g., a bacterial endosymbiont of a plant), wherein the method includes delivering to the bacteria the bacteria-derived lipid composition described herein.

In some embodiments, provided herein is a method of increasing the fitness of an insect (e.g., an insect symbiont of a plant), wherein the method includes delivering to the insect the bacteria-derived lipid composition described herein.

In some instances, the increase in symbiont fitness may manifest as an improvement in the physiology of the symbiont (e.g., improved health or survival) as a consequence of administration of the bacteria-derived lipid composition. In some instances, the fitness of an organism may be measured by one or more parameters, including, but not limited to, reproductive rate, lifespan, mobility, fecundity, body weight, metabolic rate or activity, or survival in comparison to a symbiont to which the bacteria-derived lipid composition has not been delivered. For example, the methods or compositions provided herein may be effective to improve the overall health of the symbiont or to improve the overall survival of the symbiont in comparison to a symbiont organism to which the bacteria-derived lipid composition has not been administered.

In some instances, the increase in symbiont fitness may manifest as an increase in the frequency or efficacy of a desired activity carried out by the symbiont (e.g., pollination, predation on pests, seed spreading, or breakdown of waste or organic material) in comparison to a symbiont organism to which the bacteria-derived lipid composition has not been administered.

In some instances, the increase in symbiont fitness may manifest as an increase in the production of one or more nutrients in the symbiont (e.g., vitamins, carbohydrates, amino acids, or polypeptides) in comparison to a symbiont organism to which the bacteria-derived lipid composition has not been administered.

In some instances, the increase in symbiont fitness may manifest as a decrease in the symbiont's sensitivity to a pesticidal agent and/or an increase in the symbiont's resistance to a pesticidal agent in comparison to a symbiont organism to which the bacteria-derived lipid composition has not been administered.

In some instances, the increase in symbiont fitness may manifest as a decrease in the symbiont's sensitivity to an allelochemical agent and/or an increase in the symbiont's resistance to an allelochemical agent in comparison to a symbiont organism to which the bacteria-derived lipid composition has not been administered. In some instances, the allelochemical agent is caffeine, soyacystatin N, monoterpenes, diterpene acids, or phenolic compounds. In some instances, the methods or compositions provided herein may decrease the symbiont's sensitivity to an allelochemical agent by increasing the symbiont's ability to metabolize or degrade the allelochemical agent into usable substrates.

In some instances, the methods or compositions provided herein may be effective to increase the symbiont's resistance to parasites or pathogens (e.g., fungal, bacterial, or viral pathogens; or parasitic mites (e.g., Varroa destructor mite in honeybees)) in comparison to a symbiont organism to which the bacteria-derived lipid composition has not been administered.

In some instances, the increase in symbiont fitness may manifest as other fitness advantages, such as improved tolerance to certain environmental factors (e.g., a high or low temperature tolerance), improved ability to survive in certain habitats, or an improved ability to sustain a certain diet (e.g., an improved ability to metabolize soy vs corn) in comparison to a symbiont organism to which the bacteria-derived lipid composition has not been administered.

Symbiont fitness may be evaluated using any standard methods in the art. In some instances, symbiont fitness may be evaluated by assessing an individual symbiont. Alternatively, symbiont fitness may be evaluated by assessing a symbiont population. For example, an increase in symbiont fitness may manifest as an increase in successful competition against other insects, thereby leading to an increase in the size of the symbiont population.

### Delivery to an Animal Pathogen

Provided herein are methods of delivering the bacteria-derived lipid composition described herein to an animal (e.g., human) pathogen, by contacting the pathogen with the bacteria-derived lipid composition. As used herein the term “pathogen” refers to an organism, such as a microorganism or an invertebrate, which causes disease or disease symptoms in an animal by, e.g., (i) directly infecting the animal, (ii) by producing agents that causes disease or disease symptoms in an animal (e.g., bacteria that produce pathogenic toxins and the like), and/or (iii) that elicit an immune (e.g., inflammatory response) in animals (e.g., biting insects, e.g., bedbugs). As used herein, pathogens include, but are not limited to bacteria, protozoa, parasites, fungi, nematodes, insects, viroids and viruses, or any combination thereof, wherein each pathogen is capable, either by itself or in concert with another pathogen, of eliciting disease or symptoms in animals, such as humans.

In some instances, animal (e.g., human) pathogen may be treated with the bacteria-derived lipid composition not including a heterologous functional agent. In other instances, the bacteria-derived lipid composition includes a heterologous functional agent, e.g., a heterologous therapeutic agent (e.g., antibacterial agent, antifungal agent, insecticide, nematicide, antiparasitic agent, antiviral agent, or a repellent). The methods can be useful for decreasing the fitness of an animal pathogen, e.g., to prevent or treat a pathogen infection or control the spread of a pathogen as a consequence of delivery of the bacteria-derived lipid composition.

Examples of pathogens that can be targeted in accordance with the methods described herein include bacteria (e.g., Streptococcus spp., Pneumococcus spp., Pseudomonas spp., Shigella spp, Salmonella spp., Campylobacter spp., or an Escherichia spp), fungi (Saccharomyces spp. or a Candida spp), parasitic insects (e.g., Cimex spp), parasitic nematodes (e.g., Heligmosomoides spp), or parasitic protozoa (e.g., Trichomoniasis spp).

For example, provided herein is a method of decreasing the fitness of a pathogen, the method including delivering to the pathogen a bacteria-derived lipid composition described herein, wherein the method decreases the fitness of the pathogen relative to an untreated pathogen. In some embodiments, the method includes delivering the composition to at least one habitat where the pathogen grows, lives, reproduces, feeds, or infests. In some instances of the methods described herein, the composition is delivered as a pathogen comestible composition for ingestion by the pathogen. In some instances of the methods described herein, the composition is delivered (e.g., to a pathogen) as a liquid, a solid, an aerosol, a paste, a gel, or a gas.

Also provided herein is a method of decreasing the fitness of a parasitic insect, wherein the method includes delivering to the parasitic insect the bacteria-derived lipid composition. In some instances, the method includes delivering to the parasitic insect the bacteria-derived lipid composition described herein, wherein the bacteria-derived lipid composition includes an insecticidal agent. For example, the parasitic insect may be a bedbug. Other non-limiting examples of parasitic insects are provided herein. In some instances, the method decreases the fitness of the parasitic insect relative to an untreated parasitic insect

Additionally provided herein is a method of decreasing the fitness of a parasitic nematode, wherein the method includes delivering to the parasitic nematode the bacteria-derived lipid composition described herein. In some instances, the method includes delivering to the parasitic nematode the bacteria-derived lipid composition described herein, wherein the bacteria-derived lipid composition includes a nematicidal agent. For example, the parasitic nematode is Heligmosomoides polygyrus. Other non-limiting examples of parasitic nematodes are provided herein. In some instances, the method decreases the fitness of the parasitic nematode relative to an untreated parasitic nematode.

Further provided herein is a method of decreasing the fitness of a parasitic protozoan, wherein the method includes delivering to the parasitic protozoan the bacteria-derived lipid composition described herein. In some instances, the method includes delivering to the parasitic protozoan the bacteria-derived lipid composition described herein, wherein the bacteria-derived lipid composition includes an antiparasitic agent. For example, the parasitic protozoan may be T. vaginalis. Other non-limiting examples of parasitic protozoans are provided herein. In some instances, the method decreases the fitness of the parasitic protozoan relative to an untreated parasitic protozoan.

A decrease in the fitness of the pathogen as a consequence of delivery of the bacteria-derived lipid composition can manifest in a number of ways. In some instances, the decrease in fitness of the pathogen may manifest as a deterioration or decline in the physiology of the pathogen (e.g., reduced health or survival) as a consequence of delivery of the bacteria-derived lipid composition. In some instances, the fitness of an organism may be measured by one or more parameters, including, but not limited to, reproductive rate, fertility, lifespan, viability, mobility, fecundity, pathogen development, body weight, metabolic rate or activity, or survival in comparison to a pathogen to which the bacteria-derived lipid composition has not been administered. For example, the methods or compositions provided herein may be effective to decrease the overall health of the pathogen or to decrease the overall survival of the pathogen. In some instances, the decreased survival of the pathogen is about 2%, 5%, 10%, 20%, 30%, 40%, 50%, 60%, 70%, 80%, 90%, 100%, or greater than 100% greater relative to a reference level (e.g., a level found in a pathogen that does not receive a bacteria-derived lipid composition. In some instances, the methods and compositions are effective to decrease pathogen reproduction (e.g., reproductive rate, fertility) in comparison to a pathogen to which the bacteria-derived lipid composition has not been administered. In some instances, the methods and compositions are effective to decrease other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a pathogen that does not receive a bacteria-derived lipid composition).

In some instances, the decrease in pest fitness may manifest as an increase in the pathogen's sensitivity to an antipathogen agent and/or a decrease in the pathogen's resistance to an antipathogen agent in comparison to a pathogen to which the bacteria-derived lipid composition has not been delivered.

In some instances, the decrease in pathogen fitness may manifest as other fitness disadvantages, such as a decreased tolerance to certain environmental factors (e.g., a high or low temperature tolerance), a decreased ability to survive in certain habitats, or a decreased ability to sustain a certain diet in comparison to a pathogen to which the bacteria-derived lipid composition has not been delivered.

Pathogen fitness may be evaluated using any standard methods in the art. In some instances, pest fitness may be evaluated by assessing an individual pathogen. Alternatively, pest fitness may be evaluated by assessing a pathogen population. For example, a decrease in pathogen fitness may manifest as a decrease in successful competition against other pathogens, thereby leading to a decrease in the size of the pathogen population.

The bacteria-derived lipid composition and related methods described herein are useful to decrease the fitness of an animal pathogen and thereby treat or prevent infections in animals.

### Delivery to a Pathogen Vector

Provided herein are methods of delivering the bacteria-derived lipid composition described herein to pathogen vector, such as one disclosed herein, by contacting the pathogen vector with the bacteria-derived lipid composition. As used herein, the term “vector” refers to an insect that can carry or transmit an animal pathogen from a reservoir to an animal. Exemplary vectors include insects, such as those with piercing-sucking mouthparts, as found in Hemiptera and some Hymenoptera and Diptera such as mosquitoes, bees, wasps, midges, lice, tsetse fly, fleas and ants, as well as members of the Arachnidae such as ticks and mites.

In some instances, the vector of the animal (e.g., human) pathogen may be treated with the bacteria-derived lipid composition not including a heterologous functional agent. In other instances, the bacteria-derived lipid composition includes a heterologous functional agent, e.g., a heterologous therapeutic agent (e.g., antibacterial agent, antifungal agent, insecticide, nematicide, antiparasitic agent, antiviral agent, or a repellent). The methods can be useful for decreasing the fitness of a pathogen vector, e.g., to control the spread of a pathogen as a consequence of delivery of the bacteria-derived lipid composition. Examples of pathogen vectors that can be targeted in accordance with the present methods include insects, such as those described herein.

For example, provided herein is a method of decreasing the fitness of an animal pathogen vector, the method including delivering to the vector an effective amount of the bacteria-derived lipid composition described herein, wherein the method decreases the fitness of the vector relative to an untreated vector. In some instances, the method includes delivering the composition to at least one habitat where the vector grows, lives, reproduces, feeds, or infests. In some instances, the composition is delivered as a comestible composition for ingestion by the vector. In some instances, the vector is an insect. In some instances, the insect is a mosquito, a tick, a mite, or a louse. In some instances, the composition is delivered (e.g., to the pathogen vector) as a liquid, a solid, an aerosol, a paste, a gel, or a gas.

For example, provided herein is a method of decreasing the fitness of an insect vector of an animal pathogen, wherein the method includes delivering to the vector the bacteria-derived lipid composition described herein. In some instances, the method includes delivering to the vector the bacteria-derived lipid composition, wherein the bacteria-derived lipid composition includes an insecticidal agent. For example, the insect vector may be a mosquito, tick, mite, or louse. Other non-limiting examples of pathogen vectors are provided herein. In some instances, the method decreases the fitness of the vector relative to an untreated vector.

In some instances, the decrease in vector fitness may manifest as a deterioration or decline in the physiology of the vector (e.g., reduced health or survival) as a consequence of administration of a composition. In some instances, the fitness of an organism may be measured by one or more parameters, including, but not limited to, reproductive rate, lifespan, mobility, fecundity, body weight, metabolic rate or activity, or survival in comparison to a vector organism to which the composition has not been delivered. For example, the methods or compositions provided herein may be effective to decrease the overall health of the vector or to decrease the overall survival of the vector. In some instances, the decreased survival of the vector is about 2%, 5%, 10%, 20%, 30%, 40%, 50%, 60%, 70%, 80%, 90%, 100%, or greater than 100% greater relative to a reference level (e.g., a level found in a vector that does not receive a composition). In some instances, the methods and compositions are effective to decrease vector reproduction (e.g., reproductive rate) in comparison to a vector organism to which the composition has not been delivered. In some instances, the methods and compositions are effective to decrease other physiological parameters, such as mobility, body weight, life span, fecundity, or metabolic rate, by about 2%, 5%, 10%, 20%, 30%, 40%, 50%, 60%, 70%, 80%, 90%, 100%, or greater than 100% relative to a reference level (e.g., a level found in a vector that is not delivered the composition).

In some instances, the decrease in vector fitness may manifest as an increase in the vector's sensitivity to a pesticidal agent and/or a decrease in the vector's resistance to a pesticidal agent in comparison to a vector organism to which the composition has not been delivered. In some instances, the methods or compositions provided herein may increase the vector's sensitivity to a pesticidal agent by decreasing the vector's ability to metabolize or degrade the pesticidal agent into usable substrates in comparison to a vector to which the composition has not been delivered.

In some instances, the decrease in vector fitness may manifest as other fitness disadvantages, such as decreased tolerance to certain environmental factors (e.g., a high or low temperature tolerance), decreased ability to survive in certain habitats, or a decreased ability to sustain a certain diet in comparison to a vector organism to which the composition has not been delivered. In some instances, the methods or compositions provided herein may be effective to decrease vector fitness in any plurality of ways described herein. Further, the composition may decrease vector fitness in any number of vector classes, orders, families, genera, or species (e.g., 1 vector species, 2, 3, 4, 5, 6, 7, 8, 9, 10, 15, 20, 30, 40, 50, 60, 70, 80, 90, 100, 150, 200, 200, 250, 500, or more vector species). In some instances, the composition acts on a single vector class, order, family, genus, or species.

Vector fitness may be evaluated using any standard methods in the art. In some instances, vector fitness may be evaluated by assessing an individual vector. Alternatively, vector fitness may be evaluated by assessing a vector population. For example, a decrease in vector fitness may manifest as a decrease in successful competition against other vectors, thereby leading to a decrease in the size of the vector population.

By decreasing the fitness of vectors that carry animal pathogens, the compositions provided herein are effective to reduce the spread of vector-borne diseases. The composition may be delivered to the insects using any of the formulations and delivery methods described herein, in an amount and for a duration effective to reduce transmission of the disease, e.g., reduce vertical or horizontal transmission between vectors and/or reduce transmission to animals. For example, the composition described herein may reduce vertical or horizontal transmission of a vector-borne pathogen by about 2%, 5%, 10%, 20%, 30%, 40%, 50%, 60%, 70%, 80%, 90%, 100%, or more in comparison to a vector organism to which the composition has not been delivered. As another example, the composition described herein may reduce vectorial competence of an insect vector by about 2%, 5%, 10%, 20%, 30%, 40%, 50%, 60%, 70%, 80%, 90%, 100%, or more in comparison to a vector organism to which the composition has not been delivered.

### Delivery to an Animal

Provided herein are methods of delivering the bacteria-derived lipid composition described herein to an animal cell, tissue or subject (e.g., a mammal, e.g., a human), e.g., by contacting the animal cell, tissue, subject, or a part thereof, with the bacteria-derived lipid composition. In some instances, animals may be treated with the bacteria-derived lipid composition not including a heterologous functional agent. In other instances, the bacteria-derived lipid composition includes a heterologous functional agent, e.g., a heterologous therapeutic agent (e.g., a therapeutic protein or peptide nucleic acid, or small molecule, an antibacterial agent, antifungal agent, insecticide, nematicide, antiparasitic agent, antiviral agent, or a repellent).

In one aspect, provided herein is a method of increasing the fitness of an animal (e.g., a human), the method including delivering to the animal the bacteria-derived lipid composition described herein (e.g., in an effective amount and duration) to increase the fitness of the animal relative to an untreated animal (e.g., an animal that has not been delivered the bacteria-derived lipid composition).

An increase in the fitness of the animal as a consequence of delivery of the bacteria-derived lipid composition can be determined by any method of assessing animal fitness (e.g., fitness of a mammal, e.g., fitness (e.g., health) of a human).

Provided herein is a method of modifying or increasing the fitness of an animal (e.g., a human), the method including delivering to the animal an effective amount of the bacteria-derived lipid composition described herein, wherein the method modifies the animal and thereby introduces or increases a beneficial trait in the animal (e.g., by about 1%, 2%, 5%, 10%, 20%, 30%, 40%, 50%, 60%, 70%, 80%, 90%, 100%, or more than 100%) relative to an untreated animal. In particular, the method may increase the fitness of the animal, e.g., a mammal, e.g., a human (e.g., by about 1%, 2%, 5%, 10%, 20%, 30%, 40%, 50%, 60%, 70%, 80%, 90%, 100%, or more than 100%) relative to an untreated animal.

In a further aspect, provided herein is a method of increasing the fitness of an animal (e.g., a human), the method including contacting a cell of the animal with an effective amount of the bacteria-derived lipid composition described herein, wherein the method increases the fitness of the animal, e.g., mammal, e.g., human (e.g., by about 1%, 2%, 5%, 10%, 20%, 30%, 40%, 50%, 60%, 70%, 80%, 90%, 100%, or more than 100%) relative to an untreated animal.

In certain instances, the animal is a mammal, e.g., a human. In certain instances, the animal is a livestock animal or a veterinary animal. In certain instances, the animal is a mouse.

### Application Methods

A plant described herein can be exposed to the bacteria-derived lipid composition described herein in any suitable manner that permits delivering or administering the composition to the plant. The bacteria-derived lipid composition may be delivered either alone or in combination with other active (e.g., fertilizing agents) or inactive substances and may be applied by, for example, spraying, injection (e.g., microinjection), through plants, pouring, dipping, in the form of concentrated liquids, gels, solutions, suspensions, sprays, powders, pellets, briquettes, bricks and the like, formulated to deliver an effective concentration of the bacteria-derived lipid composition. Amounts and locations for application of the compositions described herein are generally determined by the habitat of the plant, the lifecycle stage at which the plant can be targeted by the bacteria-derived lipid composition, the site where the application is to be made, and the physical and functional characteristics of the bacteria-derived lipid composition.

In some instances, the composition is sprayed directly onto a plant e.g., crops, by e.g., backpack spraying, aerial spraying, crop spraying/dusting etc. In instances where the bacteria-derived lipid composition is delivered to a plant, the plant receiving the bacteria-derived lipid composition may be at any stage of plant growth. For example, formulated bacteria-derived lipid composition can be applied as a seed-coating or root treatment in early stages of plant growth or as a total plant treatment at later stages of the crop cycle. In some instances, the bacteria-derived lipid composition may be applied as a topical agent to a plant.

Further, the bacteria-derived lipid composition may be applied (e.g., in the soil in which a plant grows, or in the water that is used to water the plant) as a systemic agent that is absorbed and distributed through the tissues of a plant. In some instances, plants or food organisms may be genetically transformed to express the bacteria-derived lipid composition.

Delayed or continuous release can also be accomplished by coating the bacteria-derived lipid composition or a composition with the bacteria-derived lipid composition(s) with a dissolvable or bioerodable coating layer, such as gelatin, which coating dissolves or erodes in the environment of use, to then make the bacteria-derived lipid composition available, or by dispersing the agent in a dissolvable or erodable matrix. Such continuous release and/or dispensing devices may be advantageously employed to consistently maintain an effective concentration of one or more of the bacteria-derived lipid composition described herein.

In some instances, the bacteria-derived lipid composition is delivered to a part of the plant, e.g., a leaf, seed, pollen, root, fruit, shoot, or flower, or a tissue, cell, or protoplast thereof. In some instances, the bacteria-derived lipid composition is delivered to a cell of the plant. In some instances, the bacteria-derived lipid composition is delivered to a protoplast of the plant. In some instances, the bacteria-derived lipid composition is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the composition is delivered to a plant embryo.

In some instances, the bacteria-derived lipid composition may be recommended for field application as an amount of bacteria-derived lipid composition per hectare (g/ha or kg/ha) or the amount of active ingredient (e.g., bacteria-derived lipid composition with or without a heterologous functional agent) or acid equivalent per hectare (kg a.i./ha or g a.i./ha). In some instances, a lower amount of heterologous functional agent in the present compositions may be required to be applied to soil, plant media, seeds plant tissue, or plants to achieve the same results as where the heterologous functional agent is applied in a composition lacking a bacteria-derived lipid composition. For example, the amount of heterologous functional agent may be applied at levels about 2, 3, 4, 5, 6, 7, 8, 9, 10, 15, 20, 30, 50, or 100-fold (or any range between about 2 and about 100-fold, for example about 2- to 10-fold; about 5- to 15-fold, about 10- to 20-fold; about 10- to 50-fold) less than the same heterologous functional agent applied in a non-bacteria-derived lipid composition, e.g., direct application of the same heterologous functional agent without bacteria-derived lipid composition(s). The bacteria-derived lipid composition can be applied at a variety of amounts per hectare, for example at about 0.0001, 0.001, 0.005, 0.01, 0.1, 1, 2, 10, 100, 1,000, 2,000, 5,000 (or any range between about 0.0001 and 5,000) kg/ha. For example, about 0.0001 to about 0.01, about 0.01 to about 10, about 10 to about 1,000, about 1,000 to about 5,000 kg/ha.

### Therapeutic Methods

The bacteria-derived lipid composition described herein can also be useful in a variety of therapeutic methods. For example, the methods and composition may be used for the prevention or treatment of pathogen infections in animals (e.g., humans). As used herein, the term “treatment” refers to administering a pharmaceutical composition to an animal for prophylactic and/or therapeutic purposes. To “prevent an infection” refers to prophylactic treatment of an animal who is not yet ill, but who is susceptible to, or otherwise at risk of, a particular disease. To “treat an infection” refers to administering treatment to an animal already suffering from a disease to improve or stabilize the animal's condition. The present methods involve delivering the bacteria-derived lipid composition described herein to an animal, such as a human.

For example, provided herein is a method of treating an animal having a fungal infection, wherein the method includes administering to the animal an effective amount of the bacteria-derived lipid composition. In some instances, the method includes administering to the animal an effective amount of the bacteria-derived lipid composition described herein, wherein the bacteria-derived lipid composition includes an antifungal agent. In some instances, the antifungal agent is a nucleic acid that inhibits expression of a gene in a fungus that causes the fungal infection.

In another aspect, provided herein is a method of treating an animal having a bacterial infection, wherein the method includes administering to the animal an effective amount of the bacteria-derived lipid composition. In some instances, the method includes administering to the animal an effective amount of the bacteria-derived lipid composition described herein, and wherein the bacteria-derived lipid composition includes an antibacterial agent. In some instances, the method decreases or substantially eliminates the bacterial infection. In some instances, the animal is a human, a veterinary animal, or a livestock animal.

The present methods are useful to treat an infection (e.g., as caused by an animal pathogen) in an animal, which refers to administering treatment to an animal already suffering from a disease to improve or stabilize the animal's condition. This may involve reducing colonization of a pathogen in, on, or around an animal by one or more pathogens (e.g., by about 1%, 2%, 5%, 10%, 20%, 30%, 40%, 50%, 60%, 70%, 80%, 90%, or 100%) relative to a starting amount and/or allow benefit to the individual (e.g., reducing colonization in an amount sufficient to resolve symptoms). In such instances, a treated infection may manifest as a decrease in symptoms (e.g., by about 1%, 2%, 5%, 10%, 20%, 30%, 40%, 50%, 60%, 70%, 80%, 90%, or 100%). In some instances, a treated infection is effective to increase the likelihood of survival of an individual (e.g., an increase in likelihood of survival by about 1%, 2%, 5%, 10%, 20%, 30%, 40%, 50%, 60%, 70%, 80%, 90%, or 100%) or increase the overall survival of a population (e.g., an increase in likelihood of survival by about 1%, 2%, 5%, 10%, 20%, 30%, 40%, 50%, 60%, 70%, 80%, 90%, or 100%). For example, the compositions and methods may be effective to “substantially eliminate” an infection, which refers to a decrease in the infection in an amount sufficient to sustainably resolve symptoms (e.g., for at least 1, 2, 3, 4, 5, 6, 7, 8, 9, 10, 11, or 12 months) in the animal.

The present methods are useful to prevent an infection (e.g., as caused by an animal pathogen), which refers to preventing an increase in colonization in, on, or around an animal by one or more pathogens (e.g., by about 1%, 2%, 5%, 10%, 20%, 30%, 40%, 50%, 60%, 70%, 80%, 90%, 100%, or more than 100% relative to an untreated animal) in an amount sufficient to maintain an initial pathogen population (e.g., approximately the amount found in a healthy individual), prevent the onset of an infection, and/or prevent symptoms or conditions associated with infection. For example, individuals may receive prophylaxis treatment to prevent a fungal infection while being prepared for an invasive medical procedure (e.g., preparing for surgery, such as receiving a transplant, stem cell therapy, a graft, a prosthesis, receiving long-term or frequent intravenous catheterization, or receiving treatment in an intensive care unit), in immunocompromised individuals (e.g., individuals with cancer, with HIV/AIDS, or taking immunosuppressive agents), or in individuals undergoing long term antibiotic therapy.

The bacteria-derived lipid composition can be formulated for administration or administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some instances, the bacteria-derived lipid composition is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of an infection described herein (when used alone or in combination with one or more other additional therapeutic agents) will depend on the type of disease to be treated, the severity and course of the disease, whether the is administered for preventive or therapeutic purposes, previous therapy, the patient's clinical history and response to the bacteria-derived lipid composition. The bacteria-derived lipid composition can be, e.g., administered to the patient at one time or over a series of treatments. For repeated administrations over several days or longer, depending on the condition, the treatment would generally be sustained until a desired suppression of disease symptoms occurs or the infection is no longer detectable. Such doses may be administered intermittently, e.g., every week or every two weeks (e.g., such that the patient receives, for example, from about two to about twenty, doses of the bacteria-derived lipid composition. An initial higher loading dose, followed by one or more lower doses may be administered. However, other dosage regimens may be useful. The progress of this therapy is easily monitored by conventional techniques and assays.

In some instances, the amount of the bacteria-derived lipid composition administered to individual (e.g., human) may be in the range of about 0.01 mg/kg to about 5 g/kg (e.g., about 0.01 mg/kg-0.1 mg/kg, about 0.1 mg/kg-1 mg/kg, about 1 mg/kg-10 mg/kg, about 10 mg/kg-100 mg/kg, about 100 mg/kg-1 g/kg, or about 1 g/kg-5 g/kg), of the individual's body weight. In some instances, the amount of the bacteria-derived lipid composition administered to individual (e.g., human) is at least 0.01 mg/kg (e.g., at least 0.01 mg/kg, at least 0.1 mg/kg, at least 1 mg/kg, at least 10 mg/kg, at least 100 mg/kg, at least 1 g/kg, or at least 5 g/kg), of the individual's body weight. The dose may be administered as a single dose or as multiple doses (e.g., 2, 3, 4, 5, 6, 7, or more than 7 doses). In some instances, the bacteria-derived lipid composition administered to the animal may be administered alone or in combination with an additional therapeutic agent. The dose of the antibody administered in a combination treatment may be reduced as compared to a single treatment. The progress of this therapy is easily monitored by conventional techniques.

### Kits

The present invention also provides a kit including a container having a bacteria-derived lipid composition described herein. The kit may further include instructional material for applying or delivering the bacteria-derived lipid composition to a plant in accordance with a method of the present invention. The skilled artisan will appreciate that the instructions for applying the bacteria-derived lipid composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

### Nucleic Acid Vaccine for Viral Infection

The bacteria-derived lipid composition can be used as a nucleic acid vaccine composition when the composition includes antigenic polypeptides to combat various viral infections.

In some embodiments, the bacteria-derived lipid composition/nucleic acid vaccine formulation (i.e., a BacLC/nucleic acid vaccine) comprises one or more polynucleotides (e.g., mRNA) encoding one or more antigenic polypeptides to combat various viral infections. The one or more polynucleotides (e.g., mRNA) encode one or more antigenic polypeptides derived from an infectious agent that causes an infectious disease, disorder, or condition, e.g., a viral infection caused by an RNA virus.

In some embodiments, the polynucleotide (e.g., mRNA) in the BacLC/nucleic acid vaccine encodes one or more wild type or engineered antigens (or an antibody to an antigen) of the various types and strains of virus as described below.

### Viral Infection.

The BacLC/nucleic acid vaccine may be suitable to combat the infectious diseases, disorders, or conditions associated with viral infections including, but not limited to, acute febrile pharyngitis, pharyngoconjunctival fever, epidemic keratoconjunctivitis, infantile gastroenteritis, coxsackie infections, infectious mononucleosis, burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, cytomegalic inclusion disease, kaposi sarcoma, multicentric castleman disease, primary effusion lymphoma, AIDS, influenza, reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, poliomyelitis, rabies, bronchiolitis, pneumonia, influenza-like syndrome, severe bronchiolitis with pneumonia, german measles, congenital rubella, varicella, and herpes zoster.

Exemplary viral infectious agents include, but are not limited to, a strain of virus selected from the group consisting of: adenovirus; Herpes simplex, type 1; Herpes simplex, type 2; encephalitis virus, papillomavirus, Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus; Human immunodeficiency virus (HIV); Influenza virus, type A or B; Guanarito virus; Junin virus; Lassa virus; Machupo virus; SabiA virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Hantavirus, Middle East Respiratory Coronavirus; Chikungunya virus or Banna virus.

The infectious agent may be a strain of virus selected from the group consisting of the virus from the following table.

Adenovirus
Banna virus
BK virus
Chikungunya Virus
Coltivirus
coxsackievirus
Crimean-Congo hemorrhagic fever virus
Dengue virus
Ebola virus
Encephalitis virus
Japanese encephalitis virus
Eastern equine encephalitis
Epstein-barr virus
Guanarito virus
Hanta virus
Herpes simplex, type 1
Herpes simplex, type 2
Human herpes virus, type 8
Herpes zoster (Varicella-zoster; Shingles) virus
Hepatitis A virus
Hepatitis B virus
Hepatitis C virus
Hepatitis D
Hepatitis E virus
Hendra virus
Human astrovirus
Human bocavirus
Human cytomegalovirus
Human Enterovirus
Human Enterovirus 68
Human Enterovirus 71
Human metapneumovirus
Human Immunodeficiency virus (HIV)
Influenza virus
Parainfluenza virus
JC virus
Junin virus
Lassa virus
Machupo virus
Marburg virus
Measles virus
Mumps virus
Nipah virus
Norwalk virus
Orbivirus
papillomavirus
Human papillomavirus (HPV)
Parvovirus B19
polio virus
Rabies virus
Rhinovirus
Rotavirus
Rubella virus
Respiratory syncytial virus (RSV)
Severe acute respiratory syndrome (SARS) virus
Middle East Respiratory Syndrome Corona (MERS) Virus
SARS-CoV-2
Smallpox
Sabiá virus
Vesicular exanthernavirus
West Nile virus
Yellow Fever virus
Zika virus

Other suitable viral infections and viral infectious agents are described in U.S. Patent Application Publication No. US2019/0015501 and U.S. Pat. No. 11,007,260, both of which are incorporated by reference in their entirety.

### A Mosquito-Borne Virus

Dengue. In some embodiments, the polynucleotide (e.g., mRNA) in the BacLC/nucleic acid vaccine encodes a strain of dengue virus (a flavi virus). In some embodiments, the polynucleotide (e.g., mRNA) encodes the E protein domain III (DENV1-4 tandem mRNA), the E protein domain 1/11 hinge region (DENV1-4 individual mRNAs), the prM protein (DENV1-4 tandem or individual mRNAs) and the C protein (DENV1-4 tandem or single mRNAs).

Chikungunya virus. In some embodiments, the polynucleotide (e.g., mRNA) in the BacLC/nucleic acid vaccine encodes a strain of Chikungunya virus. In some embodiments, the antigenic polypeptide encodes Chikungunya envelope and/or capsid antigenic polypeptide selected from the group consisting of C, E1, E2, E3, 6K, and C-E3-E2-6K-E1.

In some embodiments, the polynucleotide (e.g., mRNA) in the BacLC/nucleic acid vaccine encodes a Chikungunya polypeptide selected from the following strains and isolates: TA53, SA76, UG82, 37997, IND-06, Ross, S27, M-713424, E1-A226V, E1-T98, IND-63-WB1, Gibbs 63-263, TH35, 1-634029, AF15561, IND-73-MH5, 653496, C0392-95, P0731460, MY0211MR/06/BP, SV0444-95, K0146-95, TSI-GSD-218-VR1, TSI-GSD-218, M127, M125, 6441-88, MY0031MR/06/BP, MY0021MR/06/BP, TR206/H804187, MY/06/37348, MY/06/37350, NC/2011-568, 1455-75, RSU1, 0706aTw, InDRE51CHIK, PR-S4, AMA2798/H804298, Hu/85/NR/001, PhH15483, 0706aTw, 0802aTw, MY0191MR/06/BP, PR-S6, PER160/H803609, 99659, JKT23574, 0811 aTw, CHIK/SBY6/10, 2001908323-BDG E1, 2001907981-BDG E1, 2004904899-BDG E1, 2004904879-BDG E1, 2003902452-BDG E1, DH 130003, 0804aTw, 2002918310-BDG E1, JC2012, chik-sy, 3807, 3462, Yap 13-2148, PR-S5, 0802aTw, MY0191MR/06/Bp, 0706aTw, PhH15483, Hu/85/NR/001, CHIKV-13-112A, InDRE 4CHIK, 0806aTw, 0712aTw, 3412-78, Yap 13-2039, LEIV-CHIKV/Moscow/1, DH130003, and 20039.

Zika virus. In some embodiments, the polynucleotide (e.g., mRNA) in the BacLC/nucleic acid vaccine encodes a strain of Zika virus (a flavi virus). In some embodiments, the polynucleotide (e.g., mRNA) in the BacLC/nucleic acid vaccine encodes a ZIKV polypeptide from a ZIKV serotype selected from the group consisting of MR 766, SPH2015, and ACD75819.

Venezuelan equine encephalitis (VEE) virus. In some embodiments, the polynucleotide (e.g., mRNA) in the BacLC/nucleic acid vaccine encodes a strain of VEE virus.

The polynucleotide (e.g., mRNA) in the BacLC/nucleic acid vaccine can encode additional types of virus and strains of a mosquito-borne virus, or fragments thereof, as those described in U.S. Pat. No. 11,007,260, which is incorporated herein by reference in its entirety.

### Influenza

In some embodiments, the polynucleotide (e.g., mRNA) in the BacLC/nucleic acid vaccine encodes a strain of an influenza virus. In some embodiments, the polynucleotide (e.g., mRNA) in the BacLC/nucleic acid vaccine encodes a strain of Influenza A or Influenza B or combinations thereof.

In some embodiments, the strain of Influenza A or Influenza B is associated with birds, pigs, horses, dogs, humans or non-human primates.

In some embodiments, the polynucleotide (e.g., mRNA) in the BacLC/nucleic acid vaccine encodes a hemagglutinin protein or fragment thereof. In some embodiments, the hemagglutinin protein is H1, H2, H3, H4, H5, H6, H7, H8, H9, H10, H11, H12, H13, H14, H15, H16, H17, H18, or a fragment thereof. In some embodiments, the hemagglutinin protein does not comprise a head domain (HA1). In some embodiments, the hemagglutinin protein comprises a portion of the head domain (HA1). In some embodiments, the hemagglutinin protein does not comprise a cytoplasmic domain. In some embodiments, the hemagglutinin protein comprises a portion of the cytoplasmic domain.

In some embodiments, the polynucleotide (e.g., mRNA) in the BacLC/nucleic acid vaccine encodes a truncated hemagglutinin protein. In some embodiments, the truncated hemagglutinin protein comprises a portion of the transmembrane domain. In some embodiments, the virus is selected from the group consisting of H1N1, H3N2, H5N1, H7N9, and H10N8.

The polynucleotide (e.g., mRNA) in the BacLC/nucleic acid vaccine can encode additional types of virus and strains of an influenza virus, or fragments thereof, as those described in U.S. Patent Application Publication No. 2019/0015501, which is incorporated herein by reference in its entirety.

### Coronavirus

Betacoronavirus. In some embodiments, the polynucleotide (e.g., mRNA) in the BacLC/nucleic acid vaccine encodes a peptide/protein comprising a spike protein (S) of a betacoronavirus (BetaCoV), or a fragment or subunit thereof.

In some embodiments, the polynucleotide (e.g., mRNA) in the BacLC/nucleic acid vaccine comprises an open reading frame encoding a peptide/protein comprising a spike protein (S) of a betacoronavirus (BetaCoV), or a fragment or subunit thereof.

The polynucleotide (e.g., mRNA) in the BacLC/nucleic acid vaccine can encode different types of betacoronavirus and strains of a betacoronavirus, or fragments thereof, as those described in U.S. Pat. No. 10,933,127, which is incorporated herein by reference in its entirety.

Middle East respiratory syndrome coronavirus. In some embodiments, the polynucleotide (e.g., mRNA) in the BacLC/nucleic acid vaccine encodes a peptide/protein comprising a spike protein (S), a spike S1 fragment (S1), an envelope protein (E), a membrane protein (M), and/or a nucleocapsid protein (N) of a MERS coronavirus, or a fragment or variant of any one of these proteins.

The polynucleotide (e.g., mRNA) in the BacLC/nucleic acid vaccine can encode different strains of an MERS coronavirus, or fragments thereof, as those described in U.S. Patent Application Publication No. US2019/0351048, which is incorporated herein by reference in its entirety.

### SARS-CoV-2

In some embodiments, the polynucleotide (e.g., mRNA) in the BacLC/nucleic acid vaccine encodes one or more wild type or engineered antigens (or an antibody to an antigen) of SARS-CoV-2.

In some embodiments, the open reading frame of the polynucleotide (e.g., mRNA) in the BacLC/nucleic acid vaccine encodes one or more wild type or engineered antigens (or an antibody to an antigen) of SARS-CoV-2. In some embodiments, the open reading frame is codon-optimized.

In some embodiments, the polynucleotide (e.g., mRNA) in the BacLC/nucleic acid vaccine encodes a peptide/protein comprising a spike protein (S), a membrane (M) protein, an envelope (E) protein, and/or a nucleocapsid (NC) protein of a SARS-CoV-2 virus, or a fragment or variant thereof.

In some embodiments, the polynucleotide (e.g., mRNA) in the BacLC/nucleic acid vaccine encodes a peptide/protein comprising a spike protein (S) of a SARS-CoV-2 virus, or a fragment or variant thereof. In some embodiments, the polynucleotide (e.g., mRNA) in the BacLC/nucleic acid vaccine codes a peptide/protein comprising at least one or two domains a spike protein (S) of a SARS-CoV-2 virus, and less than the full length spike protein.

In some embodiments, the polynucleotide (e.g., mRNA) in the BacLC/nucleic acid vaccine comprises an open reading frame (ORF) that encodes a SARS-CoV-2 spike (S) protein having a double proline stabilizing mutation.

In some embodiments, the polynucleotide (e.g., mRNA) in the BacLC/nucleic acid vaccine (or its open reading frame) encodes a strain or variant of SARS-CoV-2 virus selected from the group consisting of:
alpha (lineage B.1.1.7, Q.1-Q.8),
beta (lineage B.1.351, B.1.351.2, B.1.351.3),
delta
gamma (lineage P.1, P.1.1, P.1.2)
epsilon (lineage B.1.427, B.1.429)
eta (lineage B.1.525)
iota (lineage B.1.526)
kappa (lineage B.1.617.1)
B.1.617.3
Lambda (lineage C.37)
mu (lineage B.1.621, B.1.621.1)
zeta (lineage P.2).
omicron

In some embodiments, the polynucleotide (e.g., mRNA) in the BacLC/nucleic acid vaccine (or its open reading frame) encodes a SARS-CoV-2 spike (S) protein and/or RBD, or fragments thereof. In some embodiments, the S antigen and/or RBD antigen fragment thereof comprises one or more mutations within the RBD selected from the group consisting of: K417N or K417T, N439N, N440K, G446V, L452R, Y453F, S477G or S477N, E484Q or E484K, F490S, N501S or N501Y, D614G, Q677P or Q677H, P681H or P681R.

In some embodiments, the polynucleotide (e.g., mRNA) in the BacLC/nucleic acid vaccine (or its open reading frame) encodes a SARS-CoV-2 spike (S) protein and/or RBD, or fragments thereof. In some embodiments, the S antigen comprises a mutation which stabilizes the Spike trimer, including e.g., the K986P and V987P mutations (S-2P variant) and other proline substitutions, in particular F817P, A892P, A899P and A942P, which can be combined together to obtain a multiple proline variant, in particular hexaproline variant (HexaPro).

In some embodiments, the polynucleotide (e.g., mRNA) in the BacLC/nucleic acid vaccine (or its open reading frame) encodes a SARS-CoV-2 spike (S) protein and/or RBD, or fragments thereof. In some embodiments, the S antigen comprises one or more mutations selected from the group consisting of: the substitutions L18F, T20N, P26S, D80A, D138Y, R190S, D215G, A570D, D614G, H655Y, P681H, A701V, T7161, S982A, T10271, D1118H and V1176F; and the deletions delta 69-70, delta 144, delta 242-244, and delta 246-252.

In some embodiments, the polynucleotide (e.g., mRNA) in the BacLC/nucleic acid vaccine (or its open reading frame) encodes a SARS-CoV-2 spike (S) protein and/or RBD, or fragments thereof. In some embodiments, the S antigen or RBD antigen fragment thereof comprises the following mutations: N501Y; E484K and N501Y; K417T or K417N, E484K and N501Y; K417N, N439N, Y453F, S477N, E484K, F490S, and N501Y; K417N, N439N, L452R, S477N, E484K, F490S, and N501Y.

Additional mutations may be found in WO 2021/154763A1, which is incorporated by reference in its entirety.

### Nucleic Acid Sequences

In some embodiments, the antigenic polypeptide encoded by the polynucleotide is a corona virus, or a fragment or subunit thereof. In some embodiments, the antigenic polypeptide is spike protein (S) of a MERS virus (MERS-CoV), a SARS virus (SARS-CoV), or a fragment or subunit thereof.

In some embodiments, the antigenic polypeptide is a SARS virus, or a fragment or subunit thereof. The antigenic polypeptide may be a SARS-CoV-2 spike protein or a SARS-CoV-2 spike glycoprotein.

In some embodiments, the polynucleotide may be a mRNA, an siRNA or siRNA precursor, a microRNA (miRNA) or miRNA precursor, a plasmid, a Dicer substrate small interfering RNA (dsiRNA), a short hairpin RNA (shRNA), an asymmetric interfering RNA (aiRNA), a peptide nucleic acid (PNA), a morpholino, a locked nucleic acid (LNA), a piwi-interacting RNA (piRNA), a ribozyme, a deoxyribozyme (DNAzyme), an aptamer, a circular RNA (circRNA), a guide RNA (gRNA), or a DNA molecule encoding any of these RNAs. In one embodiment, the polynucleotide is an mRNA.

In some embodiments, the polynucleotide encodes a coronavirus antigen variant (e.g., variant trimeric spike protein, such as a stabilized prefusion spike protein). Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently, a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of coronavirus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the coronavirus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

In some embodiments, the antigenic polypeptide is a structural protein. In some embodiments, the antigenic polypeptide is a spike protein, an envelope protein, a nucleocapsid protein, or a membrane protein. In some embodiments, the antigenic polypeptide is a stabilized prefusion spike protein. In some embodiments, the mRNA comprises an open reading frame that encodes a variant trimeric spike protein. The trimeric spike protein, for example, may comprise a stabilized prefusion spike protein. In some embodiments, the stabilized prefusion spike protein a double proline (S2P) mutation.

In some embodiments, the polynucleotide (e.g., mRNA) having an open reading frame (ORF) encoding a coronavirus antigen (e.g., variant trimeric spike protein, such as a stabilized prefusion spike protein). In some embodiments, the RNA (e.g., mRNA) further comprises a 5′ UTR, 3′ UTR, a poly(A) tail and/or a 5′ cap analog.

In some embodiments, the mRNA comprises a 5′ untranslated region (UTR) and/or a 3′ UTR.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U”

In some embodiments, the mRNA is derived from (a) a DNA molecule; or (b) an RNA molecule. In the mRNA, T is optionally substituted with U.

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. The sequences disclosed herein may further comprise additional elements, e.g., 5′ and 3′ UTRs, but that those elements, unlike the ORF, need not necessarily be present in a polynucleotide of the present disclosure.

### Stabilizing Elements

Naturally occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, the polynucleotide has an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Vims Capping Enzyme to generate the “Cap 0” structure: m7G(5′)ppp(5′)G (New England BioLabs, Ipswich, MA). Cap 1 structure may be generated using both Vaccinia Vims Capping Enzyme and a 2′-0 methyl-transferase to generate m7G(5′)ppp(5′)G-2′-O-methyl. Cap 2 structure may be generated from the Cap 1 structure followed by the 2′-0-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, the polynucleotide includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, the polynucleotide (e.g., mRNA) includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, b-Galactosidase, EGFP), or a marker or selection protein (e.g. alpha-Globin, Galactokinase and Xanthine:guanine phosphoribosyl transferase (GPT)).

In some embodiments, the polynucleotide (e.g., mRNA) includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence. In some embodiments, an RNA (e.g., mRNA) does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

The polynucleotide (e.g., mRNA) may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, the polynucleotide (e.g., mRNA) has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, the polynucleotide (e.g., mRNA) has an ORF that encodes a signal peptide fused to the coronavirus antigen. Signal peptides, comprising the N-terminal 15-60 amino acids of proteins, are typically needed for the translocation across the membrane on the secretory pathway and, thus, universally control the entry of most proteins both in eukaryotes and in prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane. A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than coronavirus antigens in nature) are known in the art and can be tested for desired properties and then incorporated into a nucleic acid of the disclosure. In some embodiments, the signal peptide may comprise those described in WO 2021/154763, which is incorporated by reference in its entirety.

### Fusion Proteins

In some embodiments, the polynucleotide (e.g., mRNA) encodes an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the coronavirus antigen. Antigenic fusion proteins, in some embodiments, retain the functional property from each original protein.

### Scaffold Moieties

The polynucleotide (e.g., mRNA), in some embodiments, encodes fusion proteins that comprise coronavirus antigens linked to scaffold moieties. In some embodiments, such scaffold moieties impart desired properties to an antigen encoded by a nucleic acid of the disclosure. For example, scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of −22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 A and 360 A diameter, corresponding to 180 or 240 protomers. In some embodiments, the coronavims antigen is fused to HBsAG or HBcAG to facilitate self-assembly of nanoparticles displaying the coronavims antigen.

In some embodiments, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Fawson D. M. et al. Nature. 1991; 349:541-544). Ferritin self-assembles into nanoparticles with robust thermal and chemical stability. Thus, the ferritin nanoparticle is well suited to carry and expose antigens.

Fumazine synthase (FS) is also well suited as a nanoparticle platform for antigen display. FS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The FS monomer is 150 amino acids long, and consists of beta-sheets along with tandem alpha-helices flanking its sides. A number of different quaternary structures have been reported for FS, illustrating its morphological versatility: from homopentamers up to symmetrical assemblies of 12 pentamers forming capsids of 150 A diameter. Even FS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

In some embodiments, the polynucleotide encodes a coronavims antigen (e.g., SARS-CoV-2 S protein) fused to a foldon domain. The foldon domain may be, for example, obtained from bacteriophage T4 fibritin (see, e.g., Tao Y, et al. Structure. 1997 Jun. 15; 5(6):789-98).

### Linkers and Cleavable Peptides

In some embodiments, the polynucleotide (e.g., mRNA) encodes more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2 A peptides, has been described in the art (see for example, Kim, J. H. et al. (2011) PLoS ONE 6:e18556). In some embodiments, the linker is an F2A linker. In some embodiments, the fusion protein contains three domains with intervening linkers, having the structure: domain-linker-domain-linker-domain.

Cleavable linkers known in the art may be used in connection with the disclosure. Exemplary such linkers include F2A linkers, T2A linkers, P2A linkers, E2A linkers (See, e.g., WO2017/127750, which is incorporated herein by reference in its entirety). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a coronavirus antigen). In some embodiments, a codon optimized sequence shares less than 90% sequence identity to a naturally occurring or wild-type sequence (e.g., a naturally occurring or wild-type mRNA sequence encoding a coronavirus antigen). In some embodiments, a codon optimized sequence shares less than 85% sequence identity to a naturally occurring or wild-type sequence (e.g., a naturally occurring or wild-type mRNA sequence encoding a coronavirus antigen). In some embodiments, a codon optimized sequence shares less than 80% sequence identity to a naturally occurring or wild-type sequence (e.g., a naturally occurring or wild-type mRNA sequence encoding a coronavirus antigen). In some embodiments, a codon optimized sequence shares less than 75% sequence identity to a naturally occurring or wild-type sequence (e.g., a naturally occurring or wild-type mRNA sequence encoding a coronavirus antigen).

In some embodiments, a codon optimized sequence shares between 65% and 85% (e.g., between about 67% and about 85% or between about 67% and about 80%) sequence identity to a naturally occurring or wild-type sequence (e.g., a naturally occurring or wild-type mRNA sequence encoding a coronavirus antigen). In some embodiments, a codon optimized sequence shares between 65% and 75% or about 80% sequence identity to a naturally occurring or wild-type sequence (e.g., a naturally occurring or wild-type mRNA sequence encoding a coronavirus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a coronavirus antigen encoded by a non-codon-optimized sequence. When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, the polynucleotide (e.g., mRNA) is not chemically modified and comprises the standard ribonucleotides consisting of adenosine, guanosine, cytosine and uridine. In some embodiments, nucleotides and nucleosides of the polynucleotide (e.g., mRNA) comprise standard nucleoside residues such as those present in transcribed RNA (e.g. A, G, C, or U). In some embodiments, nucleotides and nucleosides of the polynucleotide (e.g., mRNA) comprise standard deoxyribonucleosides such as those present in DNA (e.g. dA, dG, dC, or dT).

### Chemical Modifications

The polynucleotide (e.g., mRNA) comprises, in some embodiments, an RNA having an open reading frame encoding a coronavirus antigen, wherein the nucleic acid comprises nucleotides and/or nucleosides that can be standard (unmodified) or modified as is known in the art. In some embodiments, nucleotides and nucleosides of the polynucleotide (e.g., mRNA) comprise modified nucleotides or nucleosides. Such modified nucleotides and nucleosides can be naturally occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

The nucleic acids of the polynucleotide (e.g., mRNA) can comprise standard nucleotides and nucleosides, naturally-occurring nucleotides and nucleosides, non-naturally-occurring nucleotides and nucleosides, or any combination thereof.

Nucleic acids of the polynucleotide (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, the polynucleotide (e.g., mRNA) comprises uridine at one or more or all uridine positions of the nucleic acid. In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the polynucleotide (e.g., mRNA) may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C orA+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The polynucleotide (e.g., mRNA) may comprise one or more regions or parts that act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′ UTRs have features that play roles in translation initiation. They harbor signatures like Kozak sequences, which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′ UTR also have been known to form secondary structures which are involved in elongation factor binding.

In some embodiments, the 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219, which are incorporated herein by reference in their entirety). CMV immediate-early 1 (IE1) gene (US2014/0206753, WO2013/185069, which are incorporated herein by reference in their entirety), the sequence GGGAUCCUACC (WO2014/144196) may also be used. In another embodiment, 5′ UTR of a TOP gene is a 5′ UTR of a TOP gene lacking the 5′ TOP motif (the oligopyrimidine tract) (e.g., WO/2015/101414, WO2015/101415, WO/2015/062738, WO2015/024667, WO2015/024667; 5′ UTR element derived from ribosomal protein Large 32 (L32) gene (WO/2015/101414, WO2015/101415, WO/2015/062738), 5′ UTR element derived from the 5′ UTR of an hydroxysteroid (17-b) dehydrogenase 4 gene (HSD17B4) (WO2015/024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the polynucleotide (e.g., mRNA). When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

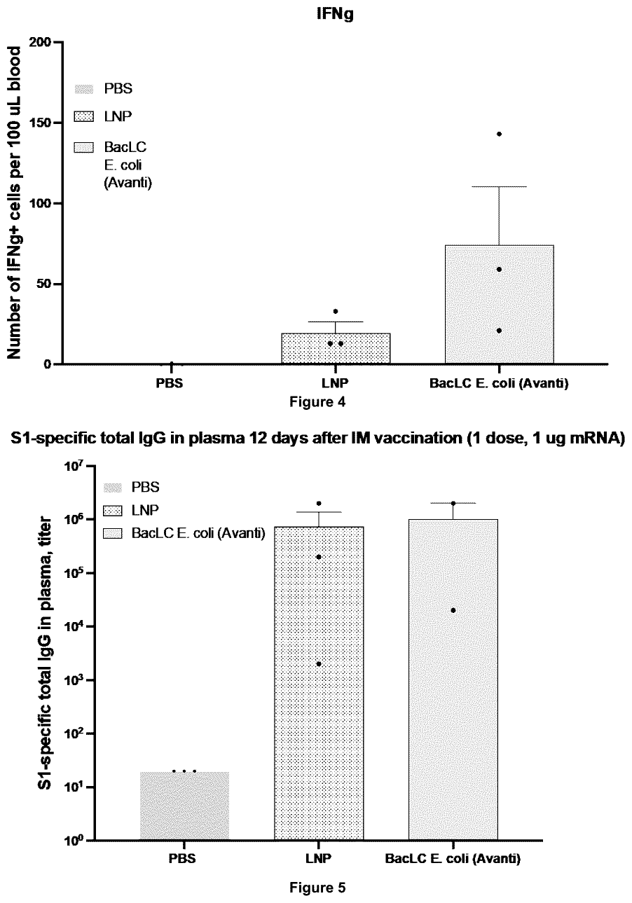

3′ UTRs may be heterologous or synthetic. With respect to 3′ UTRs, globin UTRs, including Xenopus b-globin UTRs and human b-globin UTRs are known in the art (U.S. Pat. Nos. 8,278,063, 9,012,219, US2011/0086907). A modified b-globin construct with enhanced stability in some cell types by cloning two sequential human b-globin 3′ UTRs head to tail has been developed and is well known in the art (US2012/0195936, WO2014/071963). In addition, a2-globin, al-globin, UTRs and mutants thereof are also known in the art (WO2015/101415, WO2015/024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US2014/0206753, WO2014152774), rabbit b globin and hepatitis B virus (HBV), a-globin 3′ UTR and Viral VEEV 3′ UTR sequences are also known in the art. In some embodiments, the sequence UUUGAAUU (WO2014/144196) is used. In some embodiments, 3′ UTRs of human and mouse ribosomal protein are used. Other examples include rps93′UTR (WO2015/101414), FIG. 4 (WO2015/101415), and human albumin 7 (WO2015/101415).

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 2010/0293625 and PCT/US2014/069155, which are herein incorporated by reference in their entirety. It should be understood that any UTR from any gene may be incorporated into the regions of a nucleic acid. Furthermore, multiple wild-type UTRs of any known gene may be utilized. It is also within the scope of the present disclosure to provide artificial UTRs that are not variants of wild type regions. These UTRs or portions thereof may be placed in the same orientation as in the transcript from which they were selected or may be altered in orientation or location. Hence, a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 2010/0129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins that are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest that share at least one function, structure, feature, localization, origin, or expression pattern.

The untranslated region may also include translation enhancer elements (TEE). As a non-limiting example, the TEE may include those described in US Application No. 2009/0226470, herein incorporated by reference in its entirety, and those known in the art. In vitro Transcription of RNA cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to coronavirus mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA, which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the polynucleotide (e.g., mRNA) comprises 5′ terminal cap, for example, 7mG(5′)ppp(5′)NlmpNp.

### Chemical Synthesis

Solid-phase chemical synthesis. The polynucleotide (e.g., mRNA) may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of the polynucleotide (e.g., mRNA) by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase-catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the polynucleotide (e.g., mRNA) may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheo alveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Multivalent Vaccines

The BacLC/nucleic acid vaccines, as provided herein, may include RNA or multiple RNAs encoding two or more antigens of the same or different species. In some embodiments, the BacLC/nucleic acid vaccine includes an RNA or multiple RNAs encoding two or more coronavirus antigens. In some embodiments, the RNA may encode 1, 2, 3, 4, 5, 6, 7, 8, 9, 10, 11, 12, or more coronavirus antigens.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The BacLC/nucleic acid vaccines, as provided herein, may include an RNA or multiple RNAs encoding two or more antigens of the same or different viral strains. Also provided herein are combination vaccines that include RNA encoding one or more coronavirus and one or more antigen(s) of a different organism. Thus, the vaccines of the present disclosure may be combination vaccines that target one or more antigens of the same strain/species, or one or more antigens of different strains/species, e.g., antigens which induce immunity to organisms which are found in the same geographic areas where the risk of coronavirus infection is high or organisms to which an individual is likely to be exposed to when exposed to a coronavirus.

### Vaccine Administration

In some embodiments, the BacLC/nucleic acid vaccines can be administered to a subject (e.g., a mammalian subject, such as a human subject), and the RNA polynucleotides are translated in vivo to produce an antigenic polypeptide (antigen). An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of the BacLC/nucleic acid vaccine provides an induced or boosted immune response as a function of antigen production in the cells of the subject. In some embodiments, an effective amount of the composition containing RNA polynucleotides having at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the BacLC/nucleic acid vaccines may be used for treatment or prevention of a coronavirus infection. The BacLC/nucleic acid vaccines may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

The BacLC/nucleic acid vaccines may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, the BacLC/nucleic acid vaccines may be administered intramuscularly, intranasally or intradermally, similarly to the administration of inactivated vaccines known in the art.

The BacLC/nucleic acid vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

### Kits

The present invention also provides a kit including a container having a bacteria-derived lipid composition described herein. The kit may further include instructional material for applying or delivering the bacteria-derived lipid composition to a plant in accordance with a method of the present invention. The skilled artisan will appreciate that the instructions for applying the bacteria-derived lipid composition in the methods of the present invention can be any form of instruction. Such instructions include, but are not limited to, written instruction material (such as, a label, a booklet, a pamphlet), oral instructional material (such as on an audio cassette or CD) or video instructions (such as on a video tape or DVD).

### Other Embodiments

Embodiment 1. A bacteria-derived lipid composition, comprising:
(a) a bacterial component comprising one or more lipids extracted from a bacterial source; and
(b) an ionizable lipid.

Embodiment 2. The bacteria-derived lipid composition of embodiment 1, wherein the bacterial component comprises isolated bacterial extracellular vesicles

Embodiment 3. The bacteria-derived lipid composition of embodiment 1, wherein the bacterial component is modified by reconstructing a film comprising the bacterial component in the presence of the ionizable lipid.

Embodiment 4. The bacteria-derived lipid composition of embodiment 1, wherein the bacterial component is modified by reconstructing a film comprising the purified bacteria lipids of the bacterial component with the ionizable lipid.

Embodiment 5. The bacteria-derived lipid composition of embodiment 1, wherein the ionizable lipid has one or more characteristics selected from the group consisting of:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

Embodiment 6. The bacteria-derived lipid composition of embodiment 1, wherein the bacteria-derived lipid composition further comprises a sterol and a polyethylene glycol (PEG)-lipid conjugate.

Embodiment 7. The bacteria-derived lipid composition of embodiment 6, wherein the PEG-lipid conjugate is PEG-DMG or PEG-PE.

Embodiment 8. The bacteria-derived lipid composition of embodiment 7, wherein the PEG-lipid conjugate is PEG-DMG and the PEG-DMG is PEG2000-DMG or PEG2000-PE.

Embodiment 9. The bacteria-derived lipid composition of embodiment 6, wherein the bacteria-derived lipid composition comprises:
about 20 mol % to about 50 mol % of the ionizable lipid,
about 20 mol % to about 60 mol % of the bacterial component,
about 7 mol % to about 45 mol % of the sterol, and
about 0.5 mol % to about 3 mol % of the polyethylene glycol (PEG)-lipid conjugate.

Embodiment 10. The bacteria-derived lipid composition of embodiment 9, wherein the bacteria-derived lipid composition comprises:
about 35 mol % of the ionizable lipid,
about 50 mol % of bacterial lipids,
about 12.5 mol % of the sterol, and
about 2.5 mol % the polyethylene glycol (PEG)-lipid conjugate.

Embodiment 11. The bacteria-derived lipid composition of embodiment 6, wherein the bacteria-derived lipid composition comprises ionizable lipid:bacterial lipids:sterol:PEG-lipid at a molar ratio of about 35:50:12.5:2.5.

Embodiment 12. The bacteria-derived lipid composition of embodiment 6, wherein the bacteria-derived lipid composition comprises ionizable lipid:bacterial lipids:sterol:PEG-lipid at a molar ratio of about 35:20:42.5:2.5.

Embodiment 13. The bacteria-derived lipid composition of embodiment 1, wherein the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

Embodiment 14. The bacteria-derived lipid composition of embodiment 1, wherein the ionizable lipid is

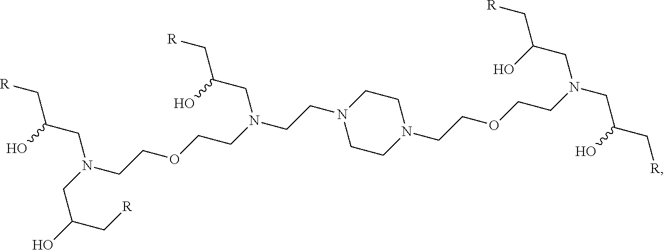

wherein each R is independently a C8-C14 alkyl group.

Embodiment 15. The bacteria-derived lipid composition of embodiment 1, wherein the bacterial source is selected from Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, and Thermoanaerobacterium.

Embodiment 16. The bacteria-derived lipid composition of embodiment 15, wherein the bacterial source is E. coli or Salmonella typhimurium.

Embodiment 17. The bacteria-derived lipid composition of embodiment 1, wherein the bacteria-derived lipid composition is a lipophilic moiety selected from the group consisting of a lipoplex, a liposome, a lipid nanoparticle, a polymer-based carrier, an exosome, a lamellar body, a micelle, and an emulsion.

Embodiment 18. The bacteria-derived lipid composition of embodiment 1, wherein the bacteria-derived lipid composition is a liposome selected from the group consisting of a cationic liposome, a nanoliposome, a proteoliposome, a unilamellar liposome, a multilamellar liposome, a ceramide-containing nanoliposome, and a multivesicular liposome.

Embodiment 19. The bacteria-derived lipid composition of embodiment 1, wherein the bacteria-derived lipid composition is a lipid nanoparticle.

Embodiment 20. The bacteria-derived lipid composition of embodiment 19, wherein the lipid nanoparticle has a size of less than about 200 nm.

Embodiment 21. The bacteria-derived lipid composition of embodiment 20, wherein the lipid nanoparticle has a size of less than about 150 nm.

Embodiment 22. The bacteria-derived lipid composition of embodiment 20, wherein the lipid nanoparticle has a size of less than about 100 nm.

Embodiment 23. The bacteria-derived lipid composition of embodiment 20, wherein the lipid nanoparticle has a size of about 85 nm to about 90 nm.

Embodiment 24. The bacteria-derived lipid composition of embodiment 19, wherein the average polydispersity index (PDI) of the lipid nanoparticle ranges from about 0.1 to about 0.4.

Embodiment 25. The bacteria-derived lipid composition of embodiment 24, wherein the average PDI of the lipid nanoparticle ranges from about 0.2 to about 0.3.

Embodiment 26. The bacteria-derived lipid composition of any one of the proceeding embodiments, wherein the bacteria-derived lipid composition further comprises one or more heterologous functional agents.

Embodiment 27. The bacteria-derived lipid composition of embodiment 26, wherein the heterologous functional agent is encapsulated by, embedded on the surface of, or conjugated to the surface of the bacteria-derived lipid composition.

Embodiment 28. The bacteria-derived lipid composition of embodiment 26, wherein the heterologous functional agent comprises a polynucleotide.

Embodiment 29. The bacteria-derived lipid composition of embodiment 28, wherein the polynucleotide is mRNA.

Embodiment 30. The bacteria-derived lipid composition of embodiment 26, wherein the bacterial derived lipid nanoparticle has a total lipid:heterologous functional agent weight ratio of about 50:1 to about 10:1.

Embodiment 31. The bacteria-derived lipid composition of embodiment 30, wherein the bacterial derived lipid nanoparticle has a total lipid:heterologous functional agent weight ratio of about 44:1 to about 24:1.

Embodiment 32. The bacteria-derived lipid composition of embodiment 30, wherein the bacterial derived lipid nanoparticle has a total lipid:heterologous functional agent weight ratio of about 40:1 to about 28:1.

Embodiment 33. The bacteria-derived lipid composition of embodiment 30, wherein the bacterial derived lipid nanoparticle has a total lipid:heterologous functional agent weight ratio of about 38:1 to about 30:1.

Embodiment 34. The bacteria-derived lipid composition of embodiment 30, wherein the bacterial derived lipid nanoparticle has a total lipid:heterologous functional agent weight ratio of about 37:1 to about 33:1.

Embodiment 35. The bacteria-derived lipid composition of any one of the proceeding embodiments, further comprising a HEPES or TRIS buffer at a pH of about 7.0 to about 8.5.

Embodiment 36. The bacteria-derived lipid composition of embodiment 35, wherein the HEPES or TRIS buffer is at a concentration of about 7 mg/mL to about 15 mg/mL.

Embodiment 37. The bacteria-derived lipid composition of embodiment 35 or 36, wherein the buffer further comprises about 2.0 mg/mL to about 4.0 mg/mL of NaCl.

Embodiment 38. The bacteria-derived lipid composition of any one of the proceeding embodiments, further comprising one or more cryoprotectants.

Embodiment 39. The bacteria-derived lipid composition of embodiment 38, wherein the one or more cryoprotectants are selected from the group consisting of sucrose, glycerol, and a combination thereof.

Embodiment 40. The bacteria-derived lipid composition of embodiment 29, wherein the bacteria-derived lipid composition comprises a combination of sucrose at a concentration of about 70 mg/mL to about 110 mg/mL and glycerol at a concentration of about 50 mg/mL to about 70 mg/mL.

Embodiment 41. The bacteria-derived lipid composition of any one of the proceeding embodiments, wherein the bacteria-derived lipid composition is a lyophilized composition.

Embodiment 42. The bacteria-derived lipid composition of embodiment 41, wherein the lyophilized bacteria-derived lipid composition comprises one or more lyoprotectants.

Embodiment 43. The bacteria-derived lipid composition of embodiment 41, wherein the lyophilized bacteria-derived lipid composition comprises a poloxamer, potassium sorbate, sucrose, or any combination thereof.

Embodiment 44. The bacteria-derived lipid composition of embodiment 43, wherein the lyophilized bacteria-derived lipid composition comprises about 0.01 to about 1.0% w/w of a poloxamer.

Embodiment 45. The bacteria-derived lipid composition of embodiment 43 or 44, wherein the poloxamer is poloxamer 188.

Embodiment 46. The bacteria-derived lipid composition of any one of embodiments 41 to 45, wherein the lyophilized bacteria-derived lipid composition comprises about 0.01 to about 1.0% w/w of polynucleotides.

Embodiment 47. The bacteria-derived lipid composition of any one of embodiments 41 to 45, wherein the lyophilized bacteria-derived lipid composition comprises about 1.0 to about 5.0% w/w lipids.

Embodiment 48. The bacteria-derived lipid composition of any one of embodiments 41 to 45, wherein the lyophilized bacteria-derived lipid composition comprises about 0.5 to about 2.5% w/w of TRIS buffer.

Embodiment 49. The bacteria-derived lipid composition of any one of embodiments 41 to 45, wherein the lyophilized bacteria-derived lipid composition comprises about 0.75 to about 2.75% w/w of NaCl.

Embodiment 50. The bacteria-derived lipid composition of any one of embodiments 41 to 45, wherein the lyophilized bacteria-derived lipid composition comprises about 85 to about 95% w/w of a sugar.

Embodiment 51. The bacteria-derived lipid composition of embodiment 50, wherein the sugar is sucrose.

Embodiment 52. The bacterial derived lipid composition of any one of embodiments 41 to 45, wherein the lyophilized bacterial derived lipid nanoparticle comprises about 1.0 to about 5.0% w/w of potassium sorbate.

Embodiment 53. A method for making a bacteria-derived lipid composition, comprising:
reconstructing (a) a bacteria component comprising one or more lipids extracted from a bacterial source in the presence of (b) an ionizable lipid, to produce the bacteria-derived lipid composition, wherein the ionizable lipid has two or more of the characteristics listed below:
   (i) at least 2 ionizable amines;
   (ii) at least 3 lipid tails, wherein each of the lipid tails is at least 6 carbon atoms in length;
   (iii) a pKa of about 4.5 to about 7.5;
   (iv) an ionizable amine and a heteroorganic group separated by a chain of at least two atoms; and
   (v) an N:P ratio of at least 10, and
loading into the bacteria-derived lipid composition with one or more heterologous functional agents.

Embodiment 54. The method of embodiment 53, wherein the reconstructing step comprises:
reconstituting a film comprising the purified bacterial lipids of the bacteria component (a) in the presence of the ionizable lipid (b) to produce the bacteria-derived lipid composition.

Embodiment 55. The method of embodiment 53, wherein the heterologous functional agent comprises a polynucleotide.

Embodiment 56. The method of embodiment 55, wherein the polynucleotide is mRNA.

Embodiment 57. The bacteria-derived lipid composition of embodiment 1, wherein the ionizable lipid has one or more characteristics selected from the group consisting of:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 3.

Embodiment 58. A method for making a bacteria-derived lipid composition, comprising:
reconstructing (a) a bacteria component comprising one or more lipids extracted from a bacterial source in the presence of (b) an ionizable lipid, to produce the bacteria-derived lipid composition, wherein the ionizable lipid has two or more of the characteristics listed below:
   (i) at least 2 ionizable amines;
   (ii) at least 3 lipid tails, wherein each of the lipid tails is at least 6 carbon atoms in length;
   (iii) a pKa of about 4.5 to about 7.5;
   (iv) an ionizable amine and a heteroorganic group separated by a chain of at least two atoms; and
   (v) an N:P ratio of at least 3, and
loading into the bacteria-derived lipid composition with one or more heterologous functional agents.

### EXAMPLES

The invention now being generally described, it will be more readily understood by reference to the following examples, which are included merely for purposes of illustration of certain aspects and embodiments of the present invention, and are not intended to limit the invention.

### Example 1: Preparation and Modification of a Bacteria-Derived Lipid Composition

This example describes the preparation of a bacterial component comprising lipids extracted from a bacterial source, and the modification of the bacterial component to form a bacteria-derived lipid composition.

Bacterial Component Comprising Lipids Extracted from a Bacterial Source

The extraction and isolation of a bacterial component comprising isolated bacterial extracellular vesicles (EVs) may be accomplished utilizing the methods disclosed in U.S. Patent Application Publication No. 2020/0254028, which is incorporated herein by reference in its entirety.

The extraction and isolation of lipids from a bacterial source may be accomplished utilizing the methods disclosed in International Application Publication No. WO 20010/120939, U.S. Pat. Nos. 7,847,113, and 8,592,188, which are incorporated herein by reference in their entirety.

Lipid extracts from a bacterial source may also be obtained from commercial sources. For instance, polar lipid extracts from E. coli can be obtained from Avanti Polar Lipids, Inc. (Alabaster, AL).

### Purification and Additional Processing of Bacterial Lipids

Purification of bacterial component using ultrafiltration combined with size-exclusion chromatography. The crude bacterial component comprising isolated bacterial extracellular vesicles (EVs) or extracted bacterial lipids fraction from the example above is concentrated using 100-kDA molecular weight cut-off (MWCO) Amicon spin filter (Merck Millipore). Subsequently, the concentrated crude bacterial component solution is loaded onto a PURE-EV size exclusion chromatography column (HansaBioMed Life Sciences Ltd) and isolated according to the manufacturer's instructions. The purified bacterial component-containing fractions are pooled after elution. Optionally, the bacterial component can be further concentrated using a 100-kDa MWCO Amicon spin filter, or by Tangential Flow Filtration (TFF).

Purification of bacterial component using an iodixanol gradient. The crude bacterial component comprising isolated bacterial extracellular vesicles (EVs) or extracted bacterial lipids fraction from the example above is purified by using an iodixanol gradient as described in Rutter and Innes, Plant Physiol. 173(1): 728-741 (2017), which is incorporated herein by reference in its entirety. To prepare discontinuous iodixanol gradients (OptiPrep; Sigma-Aldrich), solutions of 40% (v/v), 20% (v/v), 10% (v/v), and 5% (v/v) iodixanol are created by diluting an aqueous 60% OptiPrep stock solution in vesicle isolation buffer (VIB; 20 mM MES, 2 mM CaCl₂, and 0.1 M NaCl, pH 6). The gradient is formed by layering 3 ml of 40% solution, 3 mL of 20% solution, 3 mL of 10% solution, and 2 mL of 5% solution. The crude bacterial component comprising isolated bacterial extracellular vesicles (EVs) or extracted bacterial lipids fraction from the example above is centrifuged at 40,000 g for 60 minutes at 4° C. The pellet is resuspended in 0.5 ml of VIB and layered on top of the gradient. Centrifugation is performed at 100,000 g for 17 hours at 4° C. The first 4.5 ml at the top of the gradient is discarded, and subsequently 3 volumes of 0.7 ml that contain the bacterial component are collected, brought up to 3.5 mL with VIB and centrifuged at 100,000 g for 60 minutes at 4° C. The pellets are washed with 3.5 ml of VIB and repelleted using the same centrifugation conditions.

Purification of bacterial component using a sucrose gradient. The crude bacterial component comprising isolated bacterial extracellular vesicles (EVs) or extracted bacterial lipids fraction from the example above is centrifuged at 150,000 g for 90 minutes, and the bacterial component-containing pellet is resuspended in 1 ml PBS as described in Mu et al., Molecular Nutrition & Food Research. 58(7):1561-1573 (2014), which is incorporated herein by reference in its entirety. The resuspended pellet is transferred to a sucrose step gradient (8%/15%/30%/45%/60%) and centrifuged at 150,000 g for 120 minutes to produce purified bacterial component. Purified bacterial component are harvested from the 30%/45% interface.

Removal of aggregates from the bacterial component. In order to remove protein aggregates from crude bacterial component comprising isolated bacterial extracellular vesicles (EVs) or extracted bacterial lipids fraction from the example above, or from purified bacterial component from the examples above, an additional purification step can be included.

The produced bacterial component-containing solution is taken through a range of pHs to precipitate protein aggregates in solution. The pH is adjusted to 3, 5, 7, 9, or 11 with the addition of sodium hydroxide or hydrochloric acid (pH is measured using a calibrated pH probe). Once the solution is at the specified pH, it is filtered to remove particulates. Alternatively, the isolated bacterial component-containing solution can be flocculated using the addition of charged polymers, such as Polymin-P or Praestol 2640. Briefly, 2-5 g per L of Polymin-P or Praestol 2640 is added to the solution and mixed with an impeller. The solution is then filtered to remove particulates. Alternatively, aggregates are solubilized by increasing salt concentration. NaCl is added to the bacterial component-containing solution until it is at 1 mol/L. The solution is then filtered to purify the bacterial component. Alternatively, aggregates are solubilized by increasing the temperature. The isolated bacterial component-containing mixture is heated under mixing until it has reached a uniform temperature of 50° C. for 5 minutes. The mixture is then filtered to isolate the bacterial component. Alternatively, soluble contaminants from bacterial component-containing solutions are separated by size-exclusion chromatography column according to standard procedures, where the bacterial lipids elutes in the first fractions, whereas proteins and ribonucleoproteins and some lipoproteins are eluted later. The efficiency of protein aggregate removal is determined by measuring and comparing the protein concentration before and after removal of protein aggregates via BCA/Bradford protein quantification.

Preparation of a bacterial lipid (from E. coli) stock solution. Polar lipid extracts from E. coli were obtained from Avanti Polar Lipids, Inc. (Alabaster, AL). The lipids were quantitatively transferred from am-poules provided by Avanti Polar Lipids, Inc. (typically 100 mg/4 ml methylene chloride) to a 100 ml round-bottomed flask and the MeCl₂ was removed by rotary evaporation. The lipid film was dried under vacuum for at least 30 minutes, then rehydrated in the selected buffer by stirring for 30 minutes. Lipid dispersions were subjected to 10 freeze-thaw cycles, using liquid N₂ and a 37° C. water bath, then stored as aliquots, under argon, at −20° C. All steps were performed under argon in order to limit lipid exposure to air.

### Modification of the Bacterial Component to Form a Bacteria-Derived Lipid Composition

To form a bacteria-derived lipid composition, the bacteria component is modified with an ionizable lipid and cholesterol and PEG-lipid.

The bacterial lipids were extracted using Bligh-Dyer method (Bligh and Dyer, 1959, J Biolchem Physiol 37:911-917) from a concentrated solution of the bacteria component, and isolated as described above. Lipid films containing the bacterial lipids were prepared by evaporation of the solvent in the concentrated solution of the bacteria component with a stream of inert gas (e.g., nitrogen).

Ionizable lipids were added to the extracted bacterial lipid stock solution in Chloroform:Methanol (9:1) to amount to 25% or 40% (w/w) of total lipids, and lipids were resuspended by vigorous mixing. The size of the bacteria-derived lipid composition and number of particles were assessed by NanoFCM. The modification of the bacteria component with the ionizable lipids (e.g., C12-200 and MC3) enabled pH-dependent change in the surface charge of the bacteria-derived lipid composition: with decreasing pH, the surface charge of the bacteria-derived lipid composition would increase.

### Characterization of a Bacteria-Derived Lipid Composition

The particle concentration of the bacteria-derived lipid composition is determined by Nanoparticle Tracking Analysis (NTA) using a Malvern NanoSight, or by Tunable Resistive Pulse Sensing (TRPS) using an iZon qNano, following the manufacturer's instructions. The lipid concentration is determined using a fluorescent lipophilic dye, such as DiOC6 (ICN Biomedicals) as described by Rutter and Innes, Plant Physiol. 173(1): 728-741, 2017. Briefly, the bacteria-derived lipid composition is suspended in 100 ml of 10 mM DiOC6 (ICN Biomedicals) diluted with MES buffer (20 mM MES, pH 6) and 2 mM 2,29-dipyridyl disulfide, and is incubated at 37° C. for 10 minutes, washed with 3 mL of MES buffer, repelleted (40,000 g, 60 min, at 4° C.), and resuspended in fresh MES buffer. DiOC6 fluorescence intensity is measured at 485 nm excitation and 535 nm emission.

The bacteria-derived lipid composition is also characterized by electron and cryo-electron microscopy on a JEOL 1010 transmission electron microscope, following the protocol from Wu et al., Analyst. 140(2):386-406 (2015), which is incorporated herein by reference in its entirety. The size and zeta potential of the bacteria-derived lipid composition may be measured using a Malvern Zetasizer or iZon qNano, following the manufacturer's instructions. Lipids are isolated using chloroform extraction and characterized with LC-MS/MS.

### Characterization of the Stability of a Bacteria-Derived Lipid Composition

The bacteria-derived lipid composition is subjected to various storage and physiological conditions. The bacteria-derived lipid composition is suspended in water, 5% sucrose, or PBS and left for 1, 7, 30, and 180 days at −20° C., 4° C., 20° C., and 37° C. The bacteria-derived lipid composition is also suspended in water and dried using a rotary evaporator system and left for 1, 7, and 30, and 180 days at 4° C., 20° C., and 37° C. The bacteria-derived lipid composition is also suspended in water or 5% sucrose solution, flash-frozen in liquid nitrogen and lyophilized. After 1, 7, 30, and 180 days, dried and lyophilized bacteria-derived lipid composition is then resuspended in water. The previous three experiments with conditions at temperatures above 0° C. are also exposed to an artificial sunlight simulator in order to determine content stability in simulated outdoor UV conditions. The bacteria-derived lipid composition is also subjected to temperatures of 37° C., 40° C., 45° C., 50° C., and 55° C. for 1, 6, and 24 hours in buffered solutions with a pH of 1, 3, 5, 7, and 9 with or without the addition of 1 unit of trypsin or in other simulated gastric fluids.

After each of these treatments, the bacteria-derived lipid composition is bought back to 20° C., neutralized to pH 7.4, and characterized using some or all of the characterization methods described above.

### Formulation of a Bacteria-Derived Lipid Composition Using Microfluidics

In this example, a NanoAssemblr® IGNITE™ microfluidic instrument (Precision NanoSystems) is used as a model microfluidic system. This method allows formation of a bacteria-derived lipid composition via self-assembly by mixing an aqueous phase and a miscible solvent or a combination of miscible solvents containing dissolved lipids extracted from a bacterial source.

Bacterial lipid extracts from a commercial source may be used. Alternatively, lipids from a bacterial source were extracted using the Bligh-Dyer method (Bligh and Dyer, J Biolchem Physiol, 37: 911-917 (1959) from a concentrated solution of the bacteria component, and isolated as described above. The bacterial lipid extracts stock solution in chloroform:methanol (9:1), DMF:methanol (4:1), or ethanol was mixed with ionizable lipids (e.g., C12-200) and other exogenous lipids (e.g., sterols, and PEG lipids) to amount to 25% or 40% (w/w) of the total lipid, resuspended by vigorous mixing, and dried by evaporation of the solvent with a stream of inert gas (e.g., nitrogen) to prepare a lipid film.

Various organic solvents may be used to dissolve the dried lipid film: acetonitrile, acetone, ethanol, methanol, dimethylformamide, tetrahydrofuran, 1-buthanol, dimethyl sulfoxide, acetonitrile:ethanol (1:1), acetonitrile:methanol (3:1), acetone:methanol (3:1), methyl tert-butyl ether:propanol (3:2), tetrahydrofuran:methanol (3:2), dimethyl sulfoxide:methanol (3:1), and dimethylformamide:methanol (4:1). 275 μL of the organic solvent was added to 1 mg of the dried lipid film. Samples were sonicated for 10 minutes at 37° C. in a water bath sonicator.

The lipid solution (the bacteria-derived lipid composition lipids dissolved in an organic solvent or solvent combination; organic phase) was loaded into a 1 mL slip tip syringe (Becton Dickinson) and placed in a heating block set to 37° C. mounted to a NanoAssemblr® IGNITE™ microfluidic device (Precision Nanosystems). 825 μL of an aqueous phase (10 mM Citrate buffer, pH 3.2 for ionizable lipids or Milli-Q® H₂O or PBS for cationic lipids) was loaded into a 1 mL slip tip syringe (Becton Dickinson) and mounted to the microfluidic device. Aqueous and organic phases were mixed in the microfluidic device at a 3:1 volumetric ratio at a 12 mL/min flowrate. The resulting particles were dialyzed against PBS in a Slide-A-Lyzer™ G2 Cassette (20 kDa MWCO) for 4-24 hours at room temperature. Hydrodynamic diameter and polydispersity of the particles may be measured using a Zetasizer (Malvern Panalytical). The final concentration and size of the particles may be determined by NanoFCM, using concentration and size standards provided by the manufacturer.

### Example 2: Preparation and Characterization of Exemplary Bacteria-Derived Lipid Composition

This example describes the preparation of a bacteria-derived lipid composition comprising a bacterial lipid (E. coli) reconstructed with ionizable lipids, sterols, and PEG lipids, and the characterization of the bacteria-derived lipid composition, as compared to other lipid compositions (e.g., LNP).

BacLC composition. In this example, for the bacteria-derived lipid composition, polar lipid extracts from E. coli (bacterial lipid (from E. coli) stock solution prepared according to Example 1) were used as the bacterial lipids; C12-200 [1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol)] was used as the ionizable lipids; cholesterol (14:0) was used as the sterols; DMPE-PEG2k was used as model PEGylated lipids. The bacteria-derived lipid composition was prepared to result in ionizable lipid:bacterial lipid:sterol:PEG-lipid (C12-200:E. coli polar lipids:cholesterol (14:0): DMPE-PEG2k) at a molar ratio of 35:50:12.5:2.5, respectively. The above lipids were solubilized in ethanol, mixed at the above molar ratios, and diluted in ethanol (organic phase) to obtain total lipid concentration of 5.5 mM.

LNP composition. A LNP (lipid nanoparticle) formulation, as control, was prepared to result in ionizable lipid:structural lipid:sterol:PEG-lipid (C12-200:DOPE:cholesterol (14:0): DMPE-PEG2k) at a molar ratio of 35:16:46.5:2.5, respectively. DOPE: 1,2-dioleoyl-sn-glycero-3-phosphoethanolamine (Avanti Polar Lipids, Inc.). The above lipids were solubilized in ethanol, mixed at the above molar ratios, and diluted in ethanol (organic phase) to obtain total lipid concentration of 5.5 mM.

The molar ratios of various components constituting the above-described BacLC composition and LNP composition (control) are shown in FIG. 2.

The above compositions were loaded into Slide-A-Lyzer G2 dialysis cassettes (10k MWCO) and dialyzed in 200 times sample volume of 1×PBS for 4 hours at room temperature with gentle stirring. The PBS solution was refreshed, and the compositions were further dialyzed for at least 14 hours at 4° C. with gentle stirring. The dialyzed compositions were then collected and concentrated by centrifugation at 3000×g (Amicon Ultra centrifugation filters, 100k MWCO).

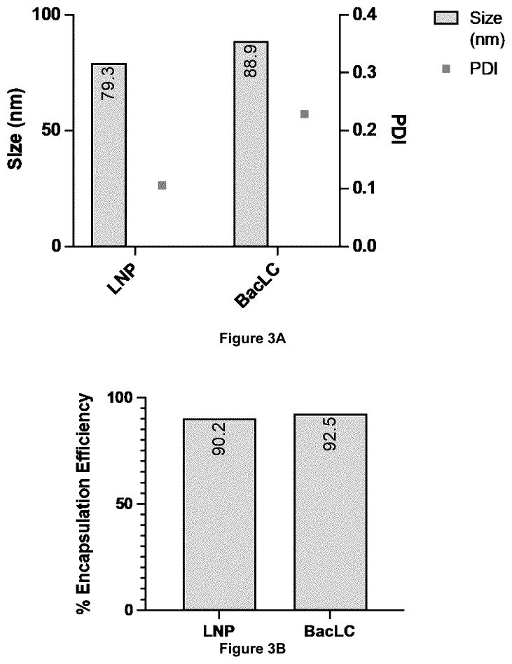

The concentrated particles were characterized for size, polydispersity, and particle concentration using Zetasizer Ultra (Malvern Panalytical). The results of the size and polydispersity of the particles of the above-described BacLC composition, as compared to those of the conventional lipid nanoparticle composition (LNP) are shown in FIG. 3A.

### Example 3: Loading a Bacteria-Derived Lipid Composition with Various Cargos

This example describes methods of loading a bacteria-derived lipid composition with various cargos (heterologous functional agents) such as small molecules, proteins, and nucleic acids.

Loading small molecules into a bacteria-derived lipid composition. A bacteria-derived lipid composition are produced as described in Example 1 or Example 2. To load small molecules into the bacteria-derived lipid composition, the bacteria-derived lipid composition is placed in PBS solution with the small molecule either in solid form or solubilized. The solution is left for 1 hour at 22° C. Alternatively, the solution is sonicated to induce poration and diffusion into the exosomes according to the protocol from Wang et al, Nature Comm. 4: Article number: 1867 (2013), which is incorporated herein by reference in its entirety. Alternatively, the bacteria-derived lipid composition is electroporated according to the protocol from Wahlgren et al, Nucl. Acids. Res. 40(17): e130 (2012), which is incorporated herein by reference in its entirety. Alternatively, the lipids in the bacteria-derived lipid composition are mixed with the small molecule solution and passed through a lipid extruder according to the protocol from Haney et al, J Contr. Rel. (2015), which is incorporated herein by reference in its entirety. Before use, the loaded bacteria-derived lipid composition may be purified using methods as described in Example 1 to remove unbound small molecules.

Loading proteins or peptides into a bacteria-derived lipid composition. A bacteria-derived lipid composition are produced as described in Example 1 or Example 2. To load proteins or peptides into the bacteria-derived lipid composition, the bacteria-derived lipid composition is placed in solution with the protein or peptide in PBS. If the protein or peptide is insoluble, pH is adjusted until it is soluble. If the protein or peptide is still insoluble, the insoluble protein or peptide is used. The solution is then sonicated to induce poration and diffusion into the bacteria-derived lipid composition according to the protocol from Wang et al, Nature Comm. 4: Article number: 1867 (2013), which is incorporated herein by reference in its entirety. Alternatively, the bacteria-derived lipid composition is electroporated according to the protocol from Wahlgren et al, Nucl. Acids. Res. 40(17): e130 (2012), which is incorporated herein by reference in its entirety. Alternatively, the lipids in the bacteria-derived lipid composition are mixed with the protein or peptide solution and passed through a lipid extruder according to the protocol from Haney et al, J Contr. Rel. (2015), which is incorporated herein by reference in its entirety. Before use, the loaded bacteria-derived lipid composition may be purified using methods as described in Example 1 to remove unbound peptides and protein. To measure loading of the protein or peptide, the Pierce Quantitative Colorimetric Peptide Assay may be used on a small sample of the loaded and unloaded bacteria-derived lipid composition.

Loading nucleic acids into a bacteria-derived lipid composition. A bacteria-derived lipid composition are produced as described in Example 1 or Example 2. To load nucleic acids into the bacteria-derived lipid composition, the bacteria-derived lipid composition is placed in solution with the nucleic acid in PBS. The solution is then sonicated to induce poration and diffusion into the bacteria-derived lipid composition according to the protocol from Wang et al, Nature Comm. 4: Article number: 1867 (2013), which is incorporated herein by reference in its entirety. Alternatively, the bacteria-derived lipid composition is electroporated according to the protocol from Wahlgren et al, Nucl. Acids. Res. 40(17): e130 (2012), which is incorporated herein by reference in its entirety. Alternatively, the lipids in the bacteria-derived lipid composition are mixed with the nucleic acid solution and passed through a lipid extruder according to the protocol from Haney et al, J Contr. Rel. (2015), which is incorporated herein by reference in its entirety. Before use, the loaded bacteria-derived lipid composition may be purified using methods as described in Example 1 to remove unbound nucleic acids. Nucleic acids that are loaded in the bacteria-derived lipid composition are quantified using either a Quant-It assay from Thermo Fisher following manufacturer's instructions, or fluorescence is quantified with a plate reader if the nucleic acids are fluorescently labeled.

### Example 4: Loading a Bacteria-Derived Lipid Composition with mRNA and Delivery into Human Cells

This example illustrates methods of loading a bacteria-derived lipid composition with mRNA and delivery of the mRNA-loaded bacteria-derived lipid composition into human cells. In this example, C12-200 is used a model ionizable lipid, and polar lipid extracts from E. coli is used as a model bacterial component. mRNA encoding Firefly Luciferase (Fluc mRNA) is used as a model mRNA, and A549 (human lung epithelial carcinoma) cells are used as a model human cell line. The bacteria-derived lipid composition containing mRNA is formulated using extrusion and microfluidics.

Modification of the bacterial component with ionizable lipids. Polar lipid extracts from E. coli can be obtained from Avanti Polar Lipids, Inc. (Alabaster, AL). Alternatively, lipids from E. coli may be extracted using the Bligh-Dyer method (Bligh and Dyer, J Biolchem Physiol, 37: 911-917 (1959) from a concentrated solution of the bacteria component, and isolated as described above in Example 1. The bacteria-derived lipid composition is prepared by adding ionizable lipids (e.g., C₁₂-200) to E. coli lipid extracts stock solution in chloroform:methanol (9:1) or DMF:methanol (4:1) to 25% or 40% (w/w) of the total lipid and resuspended by vigorous mixing before drying.

Loading of the bacteria-derived lipid composition with mRNA. For the preparation of the lipid composition with C12-200 via extrusion, mRNA dissolved in nuclease-free water is added to the dried lipid film at 35 μg of mRNA per 1 mg of bacterial lipids and is left for 1 hour at room temperature to hydrate. 0.1 M citrate buffer pH 3.2 (Teknova) is used to adjust the pH of the resuspended lipid solution to 4.5 to promote RNA entrapment. The lipid solution is then subjected to 5 freeze-thaw cycles. Subsequently, the pH of the lipid solution is brought up to pH 9 using 0.1 M bicarbonate buffer pH 10, and lipids are then subjected to 5 additional freeze-thaw cycles. The lipid composition is dialyzed overnight against PBS in a dialysis device (Spectrum®) with a 100 kDa MWCO membrane. Free RNA is removed by ultracentrifugation. The lipid composition is centrifuged for 30 minutes at 100,000 g at 4° C., supernatant is removed, and the pellets are washed with 1 mL PBS or water. Centrifugation is repeated as described above and the final pellets are resuspended in a desired buffer (e.g. PBS). The size of the vesicles and final concentration are assessed by NanoFCM.

For the preparation of the lipid composition with C12-200 via microfluidics, 1 mg of the bacterial lipids containing 25% (w/w) of C12-200 is dissolved in 275 μL of DMF:MeOH (4:1) and sonicated for 10 minutes at 37° C. in a water bath sonicator. The lipid solution (organic phase) is loaded into a 1 mL slip tip syringe (Becton Dickinson) and placed in a heating block set to 37° C. mounted to a microfluidics device. 35 μg of firefly luciferase (Fluc) mRNA is dissolved in 825 μL of Milli-Q® H₂O or PBS pH 7.4 to formulate the aqueous phase. The aqueous phase is loaded into 1 mL slip tip syringe (Becton Dickinson) and mounted to the microfluidics device (NanoAssemblr® IGNITE™, Precision Nanoystems). The aqueous and organic phases are mixed in the microfluidic device at a 3:1 volumetric ratio and at a 12 mL/min flowrate. The resulting particles were dialyzed against PBS in a Slide-A-Lyzer™ G2 Cassette (20 kDa MWCO) for 4-24 hours at room temperature. The size of the vesicles and final concentration are assessed by NanoFCM.

RNA loading is determined by the Quant-iT™ RiboGreen® assay (Thermo Scientific). The RiboGreen® assay is performed according to the manufacturer's protocol in the presence of heparin (5 mg/mL) and 1% Triton™ X-100 to lyse the mRNA-loaded lipid composition and release encapsulated cargo.

Delivery of Firefly Luciferase mRNA (Fluc) to mammalian cells (A549). A549 cells (ATCC® CCL-184™) are cultured in Dulbecco's Modified Eagle's Medium (DMEM) (4500 mg/L glucose) supplemented with 10% heat-inactivated fetal bovine serum (hiFBS, Gibco) and penicillin/streptomycin at 37° C. and 5% CO₂. Cells are passaged every 3-4 days. For the experiments, 10,000 cells per well are seeded in a 96-well plate (Corning® Costar®) in 100 μL of medium one day before the experiment. The above-prepared bacteria-derived lipid composition containing 50, 100, and 200 ng of FLuc mRNA are added to each well and incubated for 24 hours. As a positive control, cells are transfected with 100 ng of Fluc mRNA using Lipofectamine MessengerMax (Thermo Scientific), according to the manufacturer's protocol. 100 ng of free Fluc mRNA is used as negative control. Cell Viability and FLuc expression are analyzed with a CellTiter-Fluor™ Cell Viability Assay (Promega) and a Bright-Glo™ Luciferase Assay System (Promega), according to the manufacturer's protocols. Fluorescence and luminescence are quantified using a SYNERGY™ H1 plate reader (BioTek Instruments).

### Example 5: Preparation of an Exemplary mRNA-Loaded Bacteria-Derived Lipid Composition

### Manufacture and Characterization of Polynucleotides

The manufacture of polynucleotides and/or parts or regions thereof may be accomplished utilizing the methods taught in International Patent Application Publication No. WO 2014/152027, which is incorporated herein by reference in its entirety. Purification methods may include those taught in International Patent Application Publication Nos. WO2014/152030 and WO2014/152031, which are incorporated herein by reference in their entirety. Detection and characterization methods of the polynucleotides may be performed as taught in International Patent Application Publication No. WO2014/144039, which is incorporated herein by reference in its entirety. Characterization of the polynucleotides may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Patent Application Publication Nos. WO2014/144711 and WO2014/144767, which are incorporated herein by reference in their entirety.

Additional details of the mRNA design and modifications may be found in WO 2021/154763 and WO 2021/188969, which are incorporated herein by reference in their entirety.

### SARS-CoV-2 Virus Production

SARS-CoV-2 strain “Slovakia/SK-BMC5/2020”, originally provided by the European Virus Archive global (EVAg) (GISAID EPI_ISL_417879, https://www.european-virus-archive.com/virus/sars-cov-2-strain-slovakiask-bmc52020), produced and titered on Vero E6/TMPRSS2 cells, was used for hamster infection. The strain belonged to the GH clade.

Virus production was performed in T175 flasks seeded with 50×10⁶ Vero E6/TMPRSS2 cells and in a 40 mL final volume. Cell counts and viability were assessed by 0.25% trypan blue exclusion assay by ViCell apparatus. After 48 hours of infection time frame (with 0.001-0.005 MOI of SARS-CoV-2 virus), cytopathogenic effects were confirmed under microscope observation. Culture supernatant was harvested, centrifuged (5 min at 5000 g) and aliquoted (1 mL aliquots).

Virus stock TCID50 titers were determined on Vero E6/TMPRSS2 cells. About two hours before testing, cells were plated in 96-well plate at the density of 2×10⁴ cells per well in a volume of 200 μL of complete growth medium (DMEM 10% FCS). Cells were infected with serial dilutions of virus stock (8-plicates; 1st dilution 1:100; 5-fold serial dilutions) for 1 hour at 37° C. Fresh medium was added for 72 hours and a MTS/PMS assay is then performed, according to provider protocol (Promega, reference #G5430). Plates were read using an ELISA Plate reader and data recorded. Infectivity was expressed as TCID50/mL/72 h based on the Spearman-Karber formula.

SARS-CoV-2 Spike (S) mRNA Sequence

The study was designed to test the immunogenicity in mice of the candidate coronavirus vaccines comprising an mRNA of Table 1 encoding a coronavirus antigen (e.g., the spike (S) protein), such as a SARS-CoV-2 antigen.

TABLE 1
SARS-CoV-2 spike (S) mRNA sequence (with modified cap)

| Element | Description | Position |
|---|---|---|
| cap | A modified 5′-cap1 structure (m⁷G⁺m^{3′}-5′-ppp-5′-Am) | 1-2 |
| 5′-UTR | 5′-untranslated region derived from human alpha-globin RNA with an | 3-54 |
| | optimized Kozak sequence | |
| sig | S glycoprotein signal peptide (extended leader sequence), which | 55-102 |
| | guides translocation of the nascent polypeptide chain into the | |
| | endoplasmic reticulum. | |
| S protein_mut | Codon-optimized sequence encoding full-length SARS-CoV-2 spike | 103-3879 |
| | (S) glycoprotein containing mutations K986P and V987P to ensure | |
| | the S glycoprotein remains in an antigenically optimal pre-fusion | |
| | conformation; stop codons: 3874-3879 (underlined) | |
| 3′-UTR | The 3′ untranslated region comprises two sequence elements derived | 3880-4174 |
| | from the amino-terminal enhancer of split (AES) mRNA and the | |
| | mitochondrial encoded 12S ribosomal RNA to confer RNA stability | |
| | and high total protein expression. | |
| poly(A) | A 110-nucleotide poly(A)-tail consisting of a stretch of 30 adenosine | 4175-4284 |
| | residues, followed by a 10-nucleotide linker sequence and another 70 | |
| | adenosine residues. | |

An example of the coding portion of the delivered cargo S protein mRNA is as follows:

uaauacgacucacuauaagGAGAAUAAACUAGUAUUCUUCUGGUCCCCACAGACUCAGAGAGAA
CCCGCCACCAUGUUCGUGUUCCUGGUGCUGCUGCCUCUGGUGUCCAGCCAGUGUGUGA
ACCUGACCACCAGAACACAGCUGCCUCCAGCCUACACCAACAGCUUUACCAGAGGCGUG
UACUACCCCGACAAGGUGUUCAGAUCCAGCGUGCUGCACUCUACCCAGGACCUGUUCCU
GCCUUUCUUCAGCAACGUGACCUGGUUCCACGCCAUCCACGUGUCCGGCACCAAUGGCA
CCAAGAGAUUCGACAACCCCGUGCUGCCCUUCAACGACGGGGUGUACUUUGCCAGCACC
GAGAAGUCCAACAUCAUCAGAGGCUGGAUCUUCGGCACCACACUGGACAGCAAGACCCA
GAGCCUGCUGAUCGUGAACAACGCCACCAACGUGGUCAUCAAAGUGUGCGAGUUCCAGU
UCUGCAACGACCCCUUCCUGGGCGUCUACUACCACAAGAACAACAAGAGCUGGAUGGAA
AGCGAGUUCCGGGUGUACAGCAGCGCCAACAACUGCACCUUCGAGUACGUGUCCCAGC
CUUUCCUGAUGGACCUGGAAGGCAAGCAGGGCAACUUCAAGAACCUGCGCGAGUUCGU
GUUUAAGAACAUCGACGGCUACUUCAAGAUCUACAGCAAGCACACCCCUAUCAACCUCG
UGCGGGAUCUGCCUCAGGGCUUCUCUGCUCUGGAACCCCUGGUGGAUCUGCCCAUCGG
CAUCAACAUCACCCGGUUUCAGACACUGCUGGCCCUGCACAGAAGCUACCUGACACCUG
GCGAUAGCAGCAGCGGAUGGACAGCUGGUGCCGCCGCUUACUAUGUGGGCUACCUGCA
GCCUAGAACCUUCCUGCUGAAGUACAACGAGAACGGCACCAUCACCGACGCCGUGGAUU
GUGCUCUGGAUCCUCUGAGCGAGACAAAGUGCACCCUGAAGUCCUUCACCGUGGAAAAG
GGCAUCUACCAGACCAGCAACUUCCGGGUGCAGCCCACCGAAUCCAUCGUGCGGUUCCC
CAAUAUCACCAAUCUGUGCCCCUUCGGCGAGGUGUUCAAUGCCACCAGAUUCGCCUCUG
UGUACGCCUGGAACCGGAAGCGGAUCAGCAAUUGCGUGGCCGACUACUCCGUGCUGUA
CAACUCCGCCAGCUUCAGCACCUUCAAGUGCUACGGCGUGUCCCCUACCAAGCUGAACG
ACCUGUGCUUCACAAACGUGUACGCCGACAGCUUCGUGAUCCGGGGAGAUGAAGUGCG
GCAGAUUGCCCCUGGACAGACAGGCAAGAUCGCCGACUACAACUACAAGCUGCCCGACG
ACUUCACCGGCUGUGUGAUUGCCUGGAACAGCAACAACCUGGACUCCAAAGUCGGCGGC
AACUACAAUUACCUGUACCGGCUGUUCCGGAAGUCCAAUCUGAAGCCCUUCGAGCGGGA
CAUCUCCACCGAGAUCUAUCAGGCCGGCAGCACCCCUUGUAACGGCGUGGAAGGCUUCA
ACUGCUACUUCCCACUGCAGUCCUACGGCUUUCAGCCCACAAAUGGCGUGGGCUAUCAG
CCCUACAGAGUGGUGGUGCUGAGCUUCGAACUGCUGCAUGCCCCUGCCACAGUGUGCG
GCCCUAAGAAAAGCACCAAUCUCGUGAAGAACAAAUGCGUGAACUUCAACUUCAACGGCC
UGACCGGCACCGGCGUGCUGACAGAGAGCAACAAGAAGUUCCUGCCAUUCCAGCAGUUU
GGCCGGGAUAUCGCCGAUACCACAGACGCCGUUAGAGAUCCCCAGACACUGGAAAUCCU
GGACAUCACCCCUUGCAGCUUCGGCGGAGUGUCUGUGAUCACCCCUGGCACCAACACCA
GCAAUCAGGUGGCAGUGCUGUACCAGGACGUGAACUGUACCGAAGUGCCCGUGGCCAU
UCACGCCGAUCAGCUGACACCUACAUGGCGGGUGUACUCCACCGGCAGCAAUGUGUUU
CAGACCAGAGCCGGCUGUCUGAUCGGAGCCGAGCACGUGAACAAUAGCUACGAGUGCG
ACAUCCCCAUCGGCGCUGGAAUCUGCGCCAGCUACCAGACACAGACAAACAGCCCUCGG
AGAGCCAGAAGCGUGGCCAGCCAGAGCAUCAUUGCCUACACAAUGUCUCUGGGCGCCGA
GAACAGCGUGGCCUACUCCAACAACUCUAUCGCUAUCCCCACCAACUUCACCAUCAGCG
UGACCACAGAGAUCCUGCCUGUGUCCAUGACCAAGACCAGCGUGGACUGCACCAUGUAC
AUCUGCGGCGAUUCCACCGAGUGCUCCAACCUGCUGCUGCAGUACGGCAGCUUCUGCA
CCCAGCUGAAUAGAGCCCUGACAGGGAUCGCCGUGGAACAGGACAAGAACACCCAAGAG
GUGUUCGCCCAAGUGAAGCAGAUCUACAAGACCCCUCCUAUCAAGGACUUCGGCGGCUU
CAAUUUCAGCCAGAUUCUGCCCGAUCCUAGCAAGCCCAGCAAGCGGAGCUUCAUCGAGG
ACCUGCUGUUCAACAAAGUGACACUGGCCGACGCCGGCUUCAUCAAGCAGUAUGGCGAU
UGUCUGGGCGACAUUGCCGCCAGGGAUCUGAUUUGCGCCCAGAAGUUUAACGGACUGA
CAGUGCUGCCUCCUCUGCUGACCGAUGAGAUGAUCGCCCAGUACACAUCUGCCCUGCU
GGCCGGCACAAUCACAAGCGGCUGGACAUUUGGAGCAGGCGCCGCUCUGCAGAUCCCC
UUUGCUAUGCAGAUGGCCUACCGGUUCAACGGCAUCGGAGUGACCCAGAAUGUGCUGU
ACGAGAACCAGAAGCUGAUCGCCAACCAGUUCAACAGCGCCAUCGGCAAGAUCCAGGAC
AGCCUGAGCAGCACAGCAAGCGCCCUGGGAAAGCUGCAGGACGUGGUCAACCAGAAUGC
CCAGGCACUGAACACCCUGGUCAAGCAGCUGUCCUCCAACUUCGGCGCCAUCAGCUCUG
UGCUGAACGAUAUCCUGAGCAGACUGGACCCUCCUGAGGCCGAGGUGCAGAUCGACAG
ACUGAUCACAGGCAGACUGCAGAGCCUCCAGACAUACGUGACCCAGCAGCUGAUCAGAG
CCGCCGAGAUUAGAGCCUCUGCCAAUCUGGCCGCCACCAAGAUGUCUGAGUGUGUGCU
GGGCCAGAGCAAGAGAGUGGACUUUUGCGGCAAGGGCUACCACCUGAUGAGCUUCCCU
CAGUCUGCCCCUCACGGCGUGGUGUUUCUGCACGUGACAUAUGUGCCCGCUCAAGAGA
AGAAUUUCACCACCGCUCCAGCCAUCUGCCACGACGGCAAAGCCCACUUUCCUAGAGAA
GGCGUGUUCGUGUCCAACGGCACCCAUUGGUUCGUGACACAGCGGAACUUCUACGAGC
CCCAGAUCAUCACCACCGACAACACCUUCGUGUCUGGCAACUGCGACGUCGUGAUCGGC
AUUGUGAACAAUACCGUGUACGACCCUCUGCAGCCCGAGCUGGACAGCUUCAAAGAGGA
ACUGGACAAGUACUUUAAGAACCACACAAGCCCCGACGUGGACCUGGGCGAUAUCAGCG
GAAUCAAUGCCAGCGUCGUGAACAUCCAGAAAGAGAUCGACCGGCUGAACGAGGUGGCC
AAGAAUCUGAACGAGAGCCUGAUCGACCUGCAAGAACUGGGGAAGUACGAGCAGUACAU
CAAGUGGCCCUGGUACAUCUGGCUGGGCUUUAUCGCCGGACUGAUUGCCAUCGUGAUG
GUCACAAUCAUGCUGUGUUGCAUGACCAGCUGCUGUAGCUGCCUGAAGGGCUGUUGUA
GCUGUGGCAGCUGCUGCAAGUUCGACGAGGACGAUUCUGAGCCCGUGCUGAAGGGCGU
GAAACUGCACUACACAUGAUGACUCGAGCUGGUACUGCAUGCACGCAAUGCUAGCUGCC
CCUUUCCCGUCCUGGGUACCCCGAGUCUCCCCCGACCUCGGGUCCCAGGUAUGCUCCC
ACCUCCACCUGCCCCACUCACCACCUCUGCUAGUUCCAGACACCUCCCAAGCACGCAGC
AAUGCAGCUCAAAACGCUUAGCCUAGCCACACCCCCACGGGAAACAGCAGUGAUUAACC
UUUAGCAAUAAACGAAAGUUUAACUAAGCUAUACUAACCCCAGGGUUGGUCAAUUUCGU
GCCAGCCACACCCUGGAGCUAGCAAAAAAAAAAAAAAAAAAAAAAAAAAAAAAGCAUAUGA
CUAAAAAAAAAAAAAAAAAAAAAAAAAAAAAAAAAAAAAAAAAAAAAAAAAAAAAAAAAAAAA
AAAAAAAAAcggcu

Loading a Bacteria-Derived Lipid Composition with SARS-CoV-2 S mRNA

The protocols and detailed experimental procedures of the preparation and characterization of a bacteria-derived lipid composition, and loading the bacteria-derived lipid composition with mRNAs followed those described in Examples 1-4.

This example describes the formulation of a bacteria-derived lipid composition comprising a bacterial lipid (E. coli) reconstructed with ionizable lipids (C12-200), sterols (cholesterol), and PEG lipids (DMPE-PEG2k), to encapsulate mRNA (e.g., SARS-CoV-2 spike (S) mRNA sequence). As a comparative example, LNP was also formulated with the same mRNA. The bacteria-derived lipid composition (BacLC) and other lipid compositions used as comparative examples (LNP) were prepared according to Example 2. SARS-CoV-2 spike (S) mRNA sequence (described herein) was used as the loaded mRNA.

In other examples, the influenza hemagglutinin (HA) mRNA sequence (as described herein) was used as the loaded mRNA.

BacLC/mRNA formulation. An E. coli BacLC composition was formulated according to Example 2, comprising ionizable lipid:bacterial lipid:sterol:PEG-lipid (C12-200:E. coli polar lipids:cholesterol (14:0):DMPE-PEG2k) at a molar ratio of 35:50:12.5:2.5, respectively. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 15:1.

In other examples, an E. coli (Avanti) BacLC composition was formulated according to Example 2, comprising ionizable lipid:bacterial lipid:sterol:PEG-lipid (C12-200:E. coli polar lipids (purchased from Avanti):cholesterol (14:0):DMPE-PEG2k) at a molar ratio of either 35:50:12.5:2.5 or 35:20:42.5:2.5, respectively. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 15:1.

In other examples, a Salmonella BacLC composition was formulated according to Example 2, comprising ionizable lipid:bacterial lipid:sterol:PEG-lipid (C12-200:Salmonella typhimurium polar lipids:cholesterol (14:0):DMPE-PEG2k) at a molar ratio of 35:50:12.5:2.5, respectively. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 15:1.

LNP/mRNA formulation. The comparative LNP (lipid nanoparticle) composition was formulated according to Example 2, comprising ionizable lipid:structural lipid:sterol:PEG-lipid (C₁₂-200:DOPE:cholesterol (14:0):DMPE-PEG2k) at a molar ratio of 35:16:46.5:2.5, respectively. To prepare this formulation, the above lipids were solubilized in ethanol, mixed at the above molar ratios, and diluted in ethanol (organic phase) to obtain a total lipid concentration of 5.5 mM. The mRNA solution (aqueous phase) was prepared with RNAse-free water and 100 mM citrate buffer pH 3 for a final concentration of 50 mM citrate buffer. The formulations were maintained at an ionizable lipid to mRNA at an ionizable lipid nitrogen:mRNA phosphate (N:P) ratio of 15:1.

The concentrated particles were characterized for size, polydispersity, and particle concentration using Zetasizer Ultra (Malvern Panalytical). The results of the size and polydispersity of the particles of the above-described BacLC composition, as compared to those of the conventional lipid nanoparticle composition (LNP), are shown in FIG. 3A, as shown in Example 2.

The mRNA encapsulation efficiency was characterized by Quant-iT RiboGreen RNA Assay Kit (ThermoFisher Scientific). The results of the mRNA encapsulation efficiency of the particles of the above-described BacLC/mRNA formulation, as compared to those of the comparative formulation (LNP/mRNA) are shown in FIG. 3B and Table 2.

The particles were diluted to the desired mRNA concentration to get a final 10% sucrose solution in PBS. The formulations were then flash frozen in liquid nitrogen. The resulting formulations with mRNA are shown in Table 2.

TABLE 2
The formulations with mRNA

| | Ionizable:Structural | | | | | | | |
|---|---|---|---|---|---|---|---|---|
| | or natural | | | | | | | |
| | lipid:Sterol:PEG- | | | | | | | |
| | lipid | | | | | | | |
| | Molar | Ionizable | Structural or | | PEG- | mRNA | | Encapsulation |
| Formulation | ratio | lipid | natural lipid | Sterol | lipid | Cargo | N:P | efficiency (%) |
| LNP/mRNA | 35:16:46.5:2.5 | C12-200 | DOPE | Chol | DMPE- | SARS- | 15:1 | 90.2 |
| | | | | | PEG2k | CoV-2 S | | |
| BacLC/mRNA | 35:50:12.5:2.5 | C12-200 | Lipids | Chol | DMPE- | SARS- | | 92.5 |
| | | | extracted | | PEG2k | CoV-2 | | |
| | | | from E. coli | | | | | |

### Example 6: Using a mRNA-Loaded Bacteria-Derived Lipid Composition as a SARS-CoV-2 Vaccine

The BacLC/mRNA formulation (E. coli BacLC/SARS-CoV-2), and the comparative formulation (LNP/mRNA) tested in this example were prepared according to Example 5.

The design of single dose intramuscular delivery of the BacLC/mRNA formulation and evaluation is shown in Scheme 1.

As shown in Scheme 1, three mice were evaluated for each formulation. The test samples were injected to mice on DO using the intramuscular (IM) route, in one upper thigh, according to the treatment schedule in Table 3.

TABLE 3
Treatment schedule

| No. | Test | Treatment (IM) | Dosage/injection |
|---|---|---|---|
| 3 | PBS (1x dose) | 1x dose on D 0 | |
| 3 | BacLC/mRNA | 1x dose on D 0 | 1 μg mRNA |
| 3 | LNP/mRNA | 1x dose on D 0 | 1 μg mRNA |

Blood serum collection. Blood sample blood was collected on D12 and D28, from the tail vein on the mice. Blood samples were allowed to coagulate, and then centrifuged to obtain serum.

ELISPOT T cell responses evaluation in blood. T cell responses against SARS-CoV-2 antigens were evaluated on D12 in mice using IFNγ dual color ELISPOT assay. Blood cells were spun down, red blood cells were lysed, and cells were counted. Cell viability was assessed by acridine orange exclusion assay on a Luna cell counter. ELISPOT assays were run by supplementing the blood cells with 50,000 naive mouse splenocytes, which were stimulated with SARS-CoV-2/S peptides overnight. The following day, the IFNγ producing cells were evaluated using the color immunospot assay. Plates were analyzed using an ELISPOT plate reader.

ELISPOT T cell responses evaluation. T cell responses against SARS-CoV-2 antigens were evaluated on d28 in mice using an IFNγ immunospot dual color ELISPOT assay. Splenocytes were isolated on a cell strainer and filtered on before and after red blood cell lysis. Live splenocytes were counted and their viability was assessed by acridine orange exclusion assay on a Luna cell counter. ELISPOT assays were run by stimulating the cells with SARS-CoV-2/S peptides overnight. The following day, the IFNγ producing cells were evaluated using the IFNγ color immunospot assay. The plates were analyzed using an ELIPOT plate reader.

Binding antibody titers evaluation by Multiplex ELISA. The analysis of the antibody responses (IgG) against SARS-CoV-2 was performed to quantify the responses for mice antibodies targeting antigens of SARS-CoV-2 using a multiplex ELISA & neutralization assay. IgG antibody titers against SARS-CoV-2 were determined by diluting plasma in 1/10 fold dilutions. Briefly, ELISA plates were coated with S antibodies overnight. The following day, plates were blocked, samples were diluted, and all dilution were added overnight. After 3 days, detection antibodies and ELISA development were completed. Antibody titers were selected by choosing the last dilution where samples were no longer diluting.

In sum, three mice were intramuscularly administered with a single dosage of BacLC/SARS-CoV-2 (containing S mRNA 1 μg), prepared according to Example 5. Antigen specific T cells in the blood were quantified using IFNγ color ELISPOT assay at D12. Antibody response (IgG) was evaluated by Multiplex ELISA at D12. Anti-SARS-CoV-2/S T cell responses were evaluated in corresponding ELISPOT assay (IFNγ) at D28. Same assays were also carried out on mice samples (n=3) with each of those comparative formulation (LNP/mRNA, containing S mRNA 1 μg), prepared according to Example 5.

FIG. 4 shows the number of antigen-specific T cells producing IFNg in 100 uL blood of the mice at 12 days, after a single dose intramuscular delivery of BacLC E. coli (Avanti)/SARS-CoV-2 (E. coli (Avanti) BacLC, containing S mRNA 1 μg) in the mice, as compared to the mice having intramuscular delivery of a comparative formulation (LNP/mRNA containing S mRNA 1 μg), prepared according to Example 5. Control was PBS. The results indicate that a single dosage intramuscular vaccination of the E. coli BacLC/SARS-CoV-2 formulation induced systemic SARS-CoV2 Spike specific T cell response 12 days post immunization. Moreover, the E. coli BacLC/SARS-CoV-2 formulation was able to induce a higher systemic SARS-CoV2 Spike specific T cell response 12 days post immunization, as compared to the mice having the same intramuscular delivery of a comparative formulation (LNP/mRNA, containing S mRNA 1 μg) at the same dosage.

FIG. 5 show the levels of antibody (IgG) specific to S1 antigen of SARS-CoV-2 in the plasma of the mice at 12 days, after a single dose intramuscular delivery of BacLC E. coli (Avanti)/SARS-CoV-2 (E. coli (Avanti) BacLC, containing S mRNA 1 μg) in the mice, as compared to the mice having intramuscular delivery of a comparative formulation (LNP/mRNA, containing S mRNA 1 μg), prepared according to Example 5. Control was PBS. The results indicate that a single dosage intramuscular vaccination of the E. coli (Avanti) BacLC/SARS-CoV-2 formulation was able to induce a high level of S-antigen specific IgG at 12 days after vaccination in the mice. The E. coli (Avanti) BacLC/SARS-CoV-2 formulation was able to induce a higher or comparable level of S-antigen specific IgG than the LNP/mRNA.

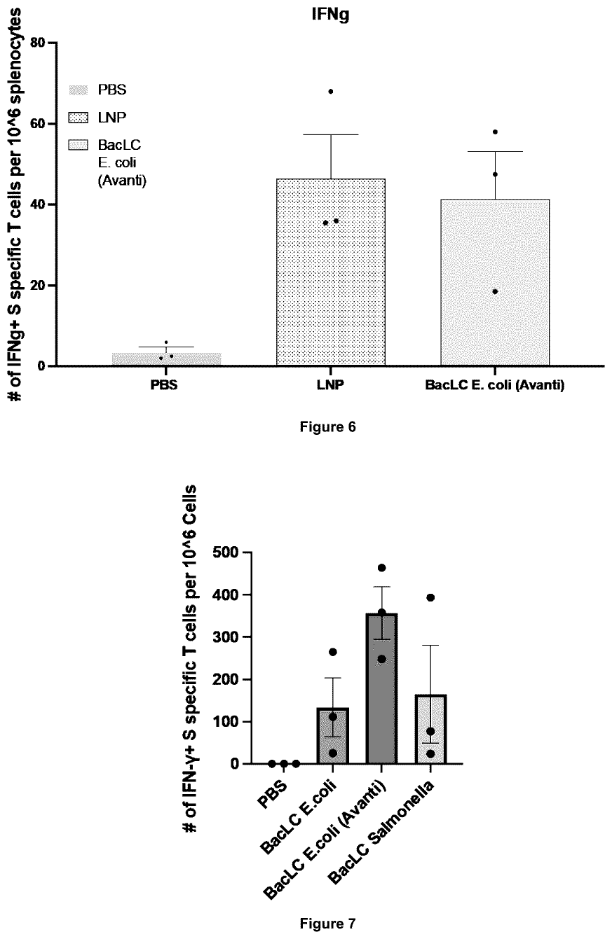

FIG. 6 shows the number of SARS-CoV-2 S-specific T cells producing cytokine IFNg per 10⁶ splenocytes in mice at 28 days, after a single dose intramuscular delivery of BacLC E. coli (Avanti)/SARS-CoV-2 (E. coli (Avanti) BacLC, containing S mRNA 1 μg) in the mice, as compared to the mice having intramuscular delivery of a comparative formulation (LNP/mRNA, containing S mRNA 1 μg), prepared according to Example 5. Control was PBS. The results indicate that a single dosage intramuscular vaccination of the E. coli BacLC/SARS-CoV-2 formulation induced SARS-CoV2 Spike specific T cell response 28 days post immunization. The E. coli (Avanti) BacLC/SARS-CoV-2 formulation was able to induce a higher or comparable level of SARS-CoV2 Spike specific T cell responses 28 days post immunization than the comparative formulation (LNP/mRNA, containing S mRNA 1 μg) at the same dosage.

### Example 7: Comparison of mRNA-Loaded Bacteria-Derived Lipid Compositions as SARS-CoV-2 Vaccine

The BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg) formulations tested in this example were prepared according to Example 5 and are listed in Table 4.

The design of single dose intramuscular delivery of the BacLC/mRNA formulations and evaluation is shown in Scheme 2.

TABLE 4
Bacterial lipid with spike mRNA

| | Ionizable:Structural or | | | | | | |
|---|---|---|---|---|---|---|---|
| | natural lipid:Sterol:PEG- | Ionizable | Structural or | | PEG- | mRNA | |
| Formulation | lipid Molar ratio | lipid | natural lipid | Sterol | lipid | Cargo | N:P |
| BacLC E. | 35:50:12.5:2.5 | C12-200 | Lipids extracted | Chol | DMPE- | SARS- | 15:1 |
| coli (Avanti) | | | from E. coli | | PEG2k | CoV-2 S | |
| | | | (Avanti) | | | | |
| BacLC E. coli | 35:50:12.5:2.5 | C12-200 | Lipids extracted | Chol | DMPE- | SARS- | |
| | | | from E. coli | | PEG2k | CoV-2 S | |
| BacLC | 35:50:12.5:2.5 | C12-200 | Lipids extracted | Chol | DMPE- | SARS- | |
| Salmonella | | | from Salmonella | | PEG2k | CoV-2 S | |

As shown in Scheme 2, three mice were evaluated for each formulation. The test samples were injected to mice on DO using the intramuscular (IM) route, in one upper thigh, according to the treatment schedule in Table 5.

TABLE 5
Treatment Schedule

| Treatment | Number of Animals | Dose Conc |
|---|---|---|
| PBS | 3 | 10 ug/20 ul; IM |
| BacLC E. coli | 3 | |
| BacLC E. coli (Avanti) | 3 | |
| BacLC Salmonella | 3 | |

Blood collection. Blood sample was collected on d7 and d12, from either the tail vein on the mice or via a terminal bleed collected through cardiac puncture. Samples were obtained via centrifuging blood at 2000 g for 10 minutes.

ELISPOT T cell responses evaluation. T cell responses against SARS-CoV-2 antigens were evaluated on d12 in a mice ELISPOT IFNγ color immunospot assay. Spleens were pushed through a cell strainer to isolate splenocytes, samples were centrifuged at 500×g for 5 minutes at 4° C. and then samples underwent red blood cell lysis. Live splenocytes were counted and their viability was assessed by acridine orange exclusion assay on a Luna cell counter. ELISPOT assays were run by stimulating the cells with SARS-CoV-2/S peptides overnight. The following day, the IFNγ producing cells were evaluated using the IFNγ color immunospot assay. The plates were analyzed using an ELIPOT plate reader.

Binding antibody titers evaluation by Multiplex ELISA. The analysis of the antibody responses (IgG) against SARS-CoV-2 was performed to quantify the responses for mice antibodies targeting antigens of SARS-CoV-2 using a multiplex ELISA & neutralization assay. IgG antibody titers against SARS-CoV-2 were determined by diluting plasma in 1/10 fold dilutions. Briefly, ELISA plates were coated with RBD antibodies overnight. The following day, plates were blocked, samples were diluted, and all dilution were added overnight. After 3 days, detection antibodies and ELISA development were completed. Antibody titers were selected by choosing the last dilution where samples were no longer diluting.

In sum, three mice were intramuscularly administered with a single dosage of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), or BacLC Salmonella/SARS-CoV-2 (Salmonella BacLC, containing S mRNA 10 μg), prepared according to Example 5. Antibody response (IgG) was evaluated by Multiplex ELISA at D12. Anti-SARS-CoV-2/S T cell responses were evaluated in corresponding ELISPOT assay (IFNγ) at D12.

FIG. 7 shows the number of SARS-CoV-2 S-specific T cells producing cytokine IFNg per 10⁶ splenocytes in the mice at 12 days after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS. The results indicate that all three BacLC formulations induced SARS-CoV-2 Spike specific T cell response 12 days post immunization.

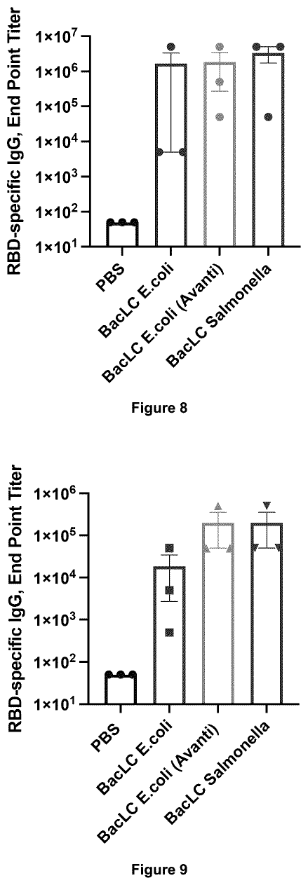

FIG. 8 shows the levels of antibody (IgG) specific to the receptor binding domain (RBD) of SARS-CoV-2 in the plasma of the mice at 12 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS. The results indicate that all three BacLC formulations induce a high level of RBD-antigen specific IgG at 12 days after vaccination in the mice.

The same formulations (Table 4) were further tested to D28. The design of single dose intramuscular delivery of the BacLC/mRNA formulations and evaluation is shown in Scheme 3.

As shown in Scheme 3, three mice were evaluated for each formulation. The test samples were injected to mice on DO using the intramuscular (IM) route, in one upper thigh, according to the treatment schedule in Table 5.

Blood collection. Blood sample was collected on d7, d13, and d28, from either the tail vein on the mice or via a terminal bleed collected through cardiac puncture. Samples were obtained via centrifuging blood at 2000 g for 10 minutes.

Binding antibody titers evaluation by Multiplex ELISA. The analysis of the antibody responses (IgG) against SARS-CoV-2 was performed to quantify the responses for mice antibodies targeting antigens of SARS-CoV-2 using a multiplex ELISA & neutralization assay. IgG antibody titers against SARS-CoV-2 were determined by diluting plasma in 1/10 fold dilutions. Briefly, ELISA plates were coated with RBD antibodies overnight. The following day, plates were blocked, samples were diluted, and all dilution were added overnight. After 3 days, detection antibodies and ELISA development were completed. Antibody titers were selected by choosing the last dilution where samples were no longer diluting.

Antibody response evaluation by Meso Scale Discovery. The analysis of the antibody responses (IgA) against SARS-CoV-2 was performed to quantify the responses for mice antibodies targeting the RBD-specific and S protein-specific antigens of SARS-CoV-2 using an MSD assay with measures corresponding to electrical chemical luminescence (ECL).

In sum, three mice were intramuscularly administered with a single dosage of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), or BacLC Salmonella/SARS-CoV-2 (Salmonella BacLC, containing S mRNA 10 μg), prepared according to Example 5. Antibody responses (IgG and IgA) in the blood and bronchoalveolar lavage fluid were evaluated by Multiplex ELISA or MSD at D28.

FIG. 9 shows the levels of antibody (IgG) specific to the receptor binding domain (RBD) of SARS-CoV-2 in the plasma of the mice at 28 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS. The results indicate that all three BacLC formulations induce a high level of RBD-antigen specific IgG at 28 days after vaccination in the mice.

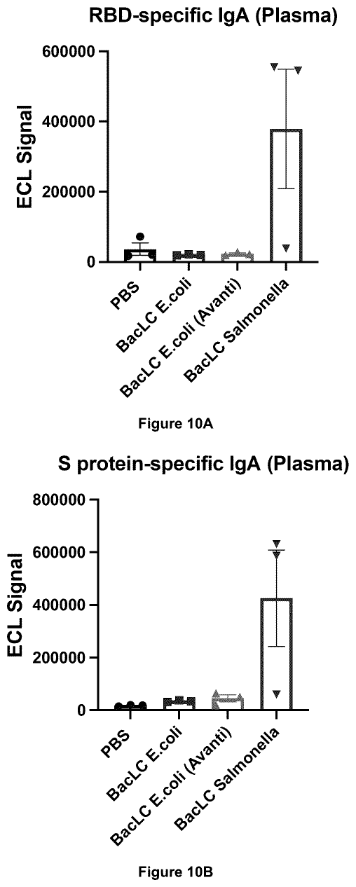

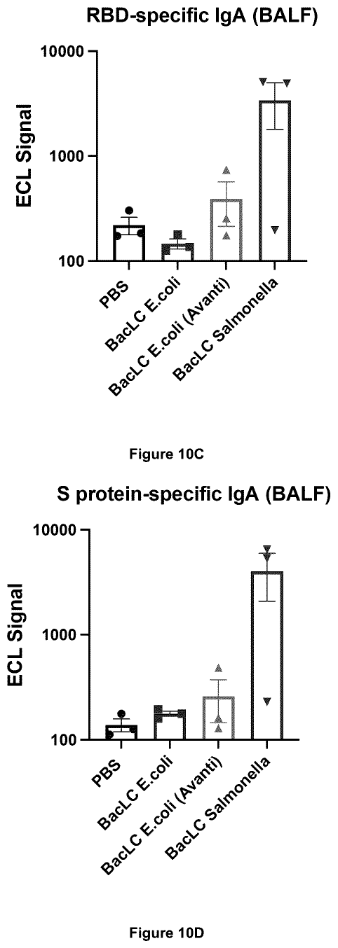

FIG. 10A-10B show the levels of antibody (IgA) specific to the receptor binding domain (RBD) or S-protein, respectively, of SARS-CoV-2 in the plasma of the mice at 28 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS. The results indicate that BacLC Salmonella/SARS-CoV-2 induces a potent immune response via high systemic levels of RBD-specific and S protein-specific IgA at 28 days after vaccination in the mice.

FIG. 10C-10D show the levels of antibody (IgA) specific to the receptor binding domain (RBD) or S-protein, respectively, of SARS-CoV-2 in the bronchoalveolar lavage fluid (BALF) of the mice at 28 days, after a single dose intramuscular delivery of BacLC E. coli/SARS-CoV-2 (E. coli BacLC, containing S mRNA 10 μg), BacLC E. coli (Avanti)/SARS-CoV-2 (E. coli (Avanti) BacLC, containing S mRNA 10 μg), and BacLC Salmonella/SARS-CoV-2 (Salmonella BacLC, containing S mRNA 10 μg), prepared according to Example 5. Control was PBS. The results indicate that BacLC Salmonella/SARS-CoV-2 induces a high potent mucosal response in the lower respiratory track via increased BALF levels of RBD-specific and S protein-specific IgA at 28 days after vaccination in the mice.

### Example 8: mRNA-Loaded Bacteria-Derived Lipid Compositions as Influenza Vaccine

The BacLC E. coli (Avanti)/Influenza formulation (E. coli (Avanti) BacLC, containing HA mRNA 10 μg) and the comparative formulations (LNP/Influenza) tested in this example were prepared according to Example 5 and are listed in Table 6. The coding portion of the delivered cargo HA is as follows:

UAAUACGACUCACUAUAAGGGGAAAUAAGAGAGAAAAGAAGAGUAAGAAGAAAUAUAA
GACCCCGGCGCCGCCACCAUGAAGGUGAAACUCCUAGUGCUGCUGUGCACCUUCACA
GCAACCUACGCCGACACCAUCUGCAUUGGUUAUCACGCGAAUAAUAGUACCGACACU
GUCGACACAGUUCUGGAGAAGAAUGUAACCGUGACCCACUCCGUCAAUUUAUUGGAG
AACGGGGGCGGGGGCAAAUAUGUUUGUAGCGCCAAACUGCGCAUGGUCACUGGAUU
GAGGAACAAGCCCUCCAAGCAGUCCCAAGGCCUGUUCGGGGCUAUUGCAGGUUUUAC
GGAGGGCGGGUGGACAGGGAUGGUGGAUGGUUGGUACGGGUACCACCACCAGAAUG
AACAAGGAUCUGGAUACGCCGCCGAUCAGAAGUCUACUCAGAAUGCUAUCAAUGGAA
UCACCAAUAAAGUUAAUUCAGUGAUAGAAAAAAUGAACACACAGUACACUGCAAUCGG
CUGCGAAUAUAACAAGUCAGAGAGAUGCAUGAAGCAGAUCGAGGAUAAGAUCGAGGA
GAUUGAGUCCAAAAUAUGGUGUUACAACGCCGAACUCCUCGUACUGCUCGAAAACGA
GAGGACACUUGAUUUUCAUGACAGUAACGUGAAAAACCUGUAUGAAAAAGUGAAAAGC
CAGCUAAAGAACAACGCUAAAGAAAUUGGCAAUGGAUGUUUUGAGUUCUACCAUAAAU
GCAAUGAUGAGUGUAUGGAGAGCGUCAAGAACGGCACGUAUGACUACCCAAAGUAUA
GCGAAGAAUCGAAGCUGAACCGGGAGAAGAUCGACGGUGUCAAGCUUGAGUCUAUGG
GCGUGUACCAAAUUGAAGGACGAUGAUAAUAGGCUGGAGCCUCGGUGGCCUAGCUUC
UUGCCCCUUGGGCCUCCCCCCAGCCCCUCCUCCCCUUCCUGCACCCGUACCCCCGU
GGUCUUUGAAUAAAGUCUGAGUGGGCGGCAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAAAA
AAAAAAAAAAAAGAAGAGC

The design of single dose intramuscular delivery of the BacLC/mRNA formulations and evaluation is shown in Scheme 4.

TABLE 6
Bacterial lipid with HA mRNA

| | Ionizable:Structural or | | | | | | |
|---|---|---|---|---|---|---|---|
| | natural lipid:Sterol:PEG- | Ionizable | Structural or natural | | | mRNA | |
| Formulation | lipid Molar ratio | lipid | lipid | Sterol | PEG-lipid | Cargo | N:P |
| BacLC E. | 35:20:42.5:2.5 | C12-200 | Lipids extracted | Chol | DMPE- | HA | 15:1 |
| coli (Avanti) | | | from E. coli (Avanti) | | PEG2k | | |
| LNP | 35:16:46.5:2.5 | C12-200 | DOPE | Chol | DMPE- | HA | |
| | | | | | PEG2k | | |

As shown in Scheme 4, five mice were evaluated for each formulation. The test samples were injected to mice on DO using the intramuscular (IM) route, with half the formulation going into each upper thigh, according to the treatment schedule in Table 7.

TABLE 7
Treatment Schedule

| Group | No. Animals | Dose Conc/Vol |
|---|---|---|
| PBS | 5 | |
| LNP | 5 | 10 ug/20 ul; IM |
| BacLC E. coli (Avanti) | 5 | |

Blood collection. Blood sample was collected on 6 h, D1, D7, and D14, from either the tail vein on the mice or via a terminal bleed collected through cardiac puncture. Samples were obtained via centrifuging blood at 2000 g for 10 minutes.

ELISPOT T cell responses evaluation. T cell responses against influenza antigens were evaluated on D14 in a mice ELISPOT IFNγ color immunospot assay. Spleens were pushed through a cell strainer to isolate splenocytes, samples were centrifuged at 500×g for 5 minutes at 4° C. and then samples underwent red blood cell lysis. Live splenocytes were counted and their viability was assessed by acridine orange exclusion assay on a Luna cell counter. ELISPOT assays were run by stimulating the cells with Influenza/HA peptides overnight. The following day, the IFNγ producing cells were evaluated using the IFNγ color immunospot assay. The plates were analyzed using an ELIPOT plate reader.

Antibody response evaluation by Multiplex ELISA. The analysis of the antibody response (IgG) against influenza was performed to quantify the responses for mice antibodies targeting the HA-specific antigens of influenza using a multiplex ELISA.

In sum, five mice were intramuscularly administered with a single dosage of BacLC E. coli (Avanti)/Influenza (E. coli (Avanti) BacLC, containing HA mRNA 10 μg), as compared to the mice having intramuscular delivery of the comparative formulation (LNP), prepared according to Example 5. Antibody responses (IgG) in the plasma were evaluated by Multiplex ELISA at D14. Anti-Influenza/HA T cell responses were evaluated in corresponding ELISPOT assay (IFNγ) at D14.

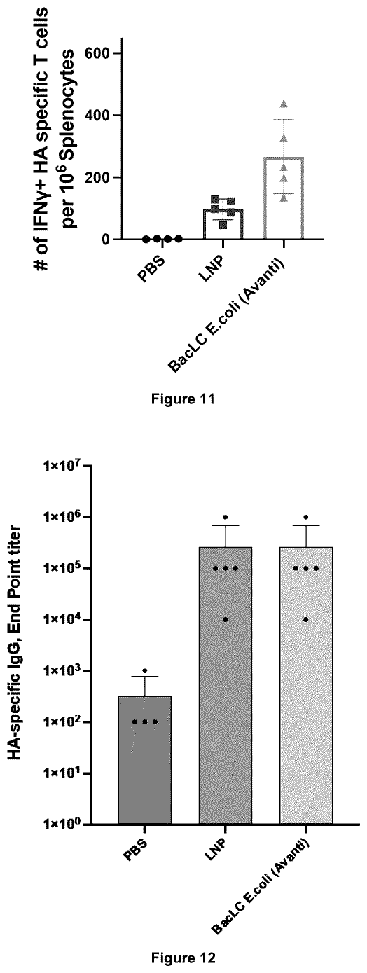

FIG. 11 shows the number of influenza HA-specific T cells producing cytokine IFNg per 10⁶ splenocytes in the mice at 14 days after a single dose intramuscular delivery of BacLC E. coli (Avanti)/Influenza (E. coli (Avanti) BacLC, containing HA mRNA 10 μg), as compared to the mice having intramuscular delivery of the comparative formulation (LNP) prepared according to Example 5. Control was PBS. The results indicate that a single dosage intramuscular vaccination of the BacLC E. coli (Avanti)/Influenza formulation induced splenic influenza HA specific T cell response 14 days post immunization. Moreover, the BacLC E. coli (Avanti)/Influenza formulation was able to induce a higher T cell response in the spleen 14 days post immunization, as compared to the mice having the same intramuscular delivery of the comparative formulation (LNP/mRNA containing HA mRNA 10 μg) at the same dosage.

FIG. 12 shows the levels of antibody (IgG) specific to the hemagglutinin (HA) of influenza in the plasma of the mice at 14 days, after a single dose intramuscular delivery of BacLC E. coli (Avanti)/Influenza (E. coli (Avanti) BacLC, containing HA mRNA 10 μg), as compared to the mice having intramuscular delivery of the comparative formulation (LNP) prepared according to Example 5. Control was PBS. The results indicate that BacLC E. coli (Avanti)/Influenza induces a potent immune response via high systemic levels of HA-specific IgG at 14 days after vaccination in the mice, at a level equivalent to the comparative formulation (LNP/mRNA containing HA mRNA 10 μg).

### Example 9: Transfection and Biodistribution of Bacteria-Derived Lipid Compositions

The BacLC E. coli (Avanti)/mRNA formulation (E. coli (Avanti) BacLC, containing CRE mRNA 10 μg) and the comparative formulations (LNP/mRNA containing CRE mRNA 10 μg) tested in this example were prepared according to Example 5 and are listed in Table 8.

The design of single dose intramuscular delivery of the BacLC/mRNA formulations and evaluation are provided herein. Four TdTomato/Ai9 mice per group (Buffer, BacLC E. coli (Avanti), and LNP) were injected intramuscularly with 10 μg/20 μL on DO. On D6, spleen and lymph nodes were collected and prepared for FACS.

TABLE 8
Bacterial lipid with CRE mRNA

| | Ionizable:Structural or | | | | | | |
|---|---|---|---|---|---|---|---|
| | natural lipid:Sterol:PEG- | Ionizable | Structural or natural | | | mRNA | |
| Formulation | lipid Molar ratio | lipid | lipid | Sterol | PEG-lipid | Cargo | N:P |
| BacLC E. | 35:20:42.5:2.5 | C12-200 | Lipids extracted from | Chol | DMPE- | CRE | 15:1 |
| coli (Avanti) | | | E. coli (Avanti) | | PEG2k | | |
| LNP | 35:16:46.5:2.5 | C12-200 | DOPE | Chol | DMPE- | CRE | |
| | | | | | PEG2k | | |

Fluorescence Activated Cell Sorting (FACS). The frequency of tdTomato+ cells were analyzed in the lymphocytes, myeloid cells, and non-immune cells collected from spleens and lymph nodes (inguinal and popliteal) of the mice. Spleens were strained through a filter, centrifuged at 500× g for 5 minutes at 4° C., then the cells underwent red blood cell lysis. The cells were centrifuged and resuspended in Fc block, incubated on ice for 10 mins, and then the antibody were added. The cells were incubated in the dark for 30 minutes on ice, washed with FACS buffer and flow cytometry was run on them. The lymph nodes were combined, digested using collagenase and DNase for 10 minutes at 37° C. and then strained. After straining, the cells were centrifuged at 500×g for 5 minutes at 4° C. before following the protocol as above from the Fc block step. Anitbodies used include, CD4, F4/80, CD31, EpCam, CD11 b, tdtomato, CD45, a live/dead stain, CD11C, CD19, CD8b and CD3. In sum, four Ai9/tdTomato mice were intramuscularly administered with a single dosage of BacLC E. coli (Avanti)/mRNA (E. coli (Avanti) BacLC, containing CRE mRNA 10 μg), as compared to the four mice having intramuscular delivery of the comparative formulation (LNP containing CRE mRNA 10 μg or buffer), prepared according to Example 5. Frequency of tdTomato+ cells as a measure of transfection were measured via FACS on spleen and lymph nodes taken six days post-dose.

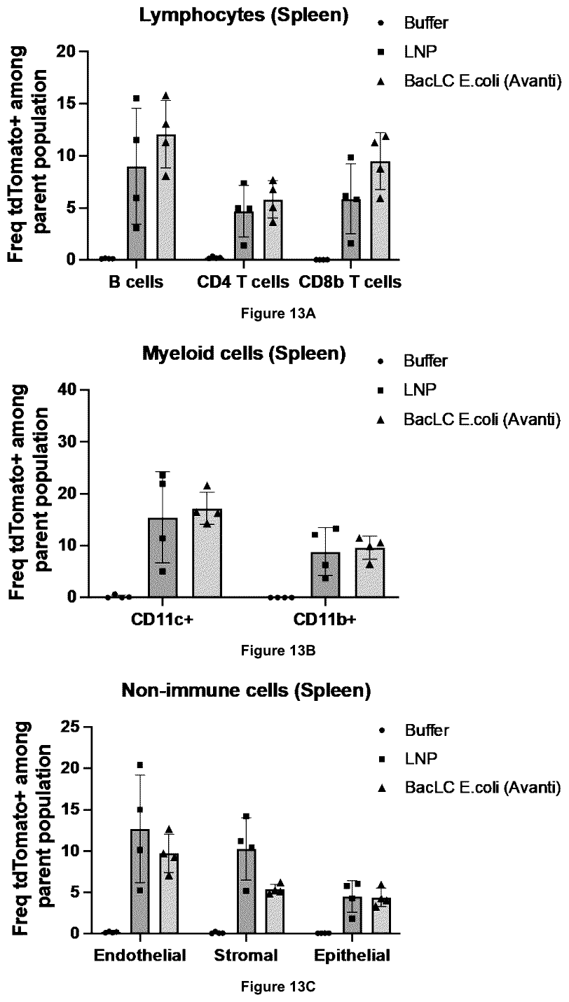

FIG. 13A-13C show the frequency of tdTomato+ lymphocytes, myeloid cells, and non-immune cells, respectively, in the spleens of Ai9 mice at 6 days after a single dose intramuscular delivery of E. coli (Avanti)/mRNA (E. coli (Avanti) BacLC, containing CRE mRNA 10 μg), as compared to the mice having intramuscular delivery of the comparative formulation (LNP) prepared according to Example 5. Control was PBS. The results indicate that BacLC E. coli (Avanti) had high transfection in splenic lymphocytes, myeloid cells, and non immune cells, at a level equivalent to the comparative formulation (LNP/mRNA containing CRE mRNA 10 μg).

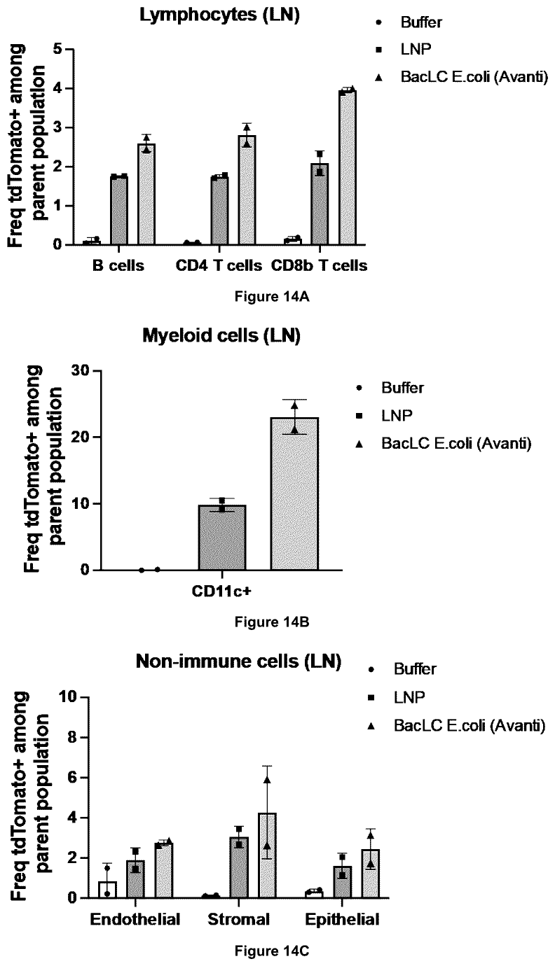

FIG. 14A-14C show the frequency of tdTomato+ lymphocytes, myeloid cells, and non-immune cells, respectively, in the lymph nodes of Ai9 mice at 6 days after a single dose intramuscular delivery of E. coli (Avanti)/mRNA (E. coli (Avanti) BacLC, containing CRE mRNA 10 μg), as compared to the mice having intramuscular delivery of the comparative formulation (LNP) prepared according to Example 5. Control was PBS. The results indicate that BacLC E. coli (Avanti) had high transfection in splenic lymphocytes, myeloid cells, and non immune cells, at a level equivalent to or higher than the comparative formulation (LNP/mRNA containing CRE mRNA 10 μg).

In addition to transfection, the biodistribution of various BacLC/mRNA formulations was tested. The BacLC E. coli/mRNA (E. coli BacLC, containing FLuc:EPO mRNA 10 μg), BacLC E. coli (Avanti)/mRNA (E. coli (Avanti) BacLC, containing FLuc:EPO mRNA 10 μg), and BacLC Salmonella/mRNA (Salmonella BacLC, containing FLuc:EPO mRNA 10 μg) formulations tested in this example were prepared according to Example 5 and are listed in Table 9.

The coding portion of the FLuc mRNA (TriLink, CleanCap FLuc mRNA) used in this example is as follows:

AUGGAGGACGCCAAGAACAUCAAGAAGGGCCCCGCCCCCUUCUACCCCC
UGGAGGACGGCACCGCCGGCGAGCAGCUGCACAAGGCCAUGAAGCGGUA
CGCCCUGGUGCCCGGCACCAUCGCCUUCACCGACGCCCACAUCGAGGUG
GACAUCACCUACGCCGAGUACUUCGAGAUGAGCGUGCGGCUGGCCGAGG
CCAUGAAGCGGUACGGCCUGAACACCAACCACCGGAUCGUGGUGUGCAG
CGAGAACAGCCUGCAGUUCUUCAUGCCCGUGCUGGGCGCCCUGUUCAUC
GGCGUGGCCGUGGCCCCCGCCAACGACAUCUACAACGAGCGGGAGCUGC
UGAACAGCAUGGGCAUCAGCCAGCCCACCGUGGUGUUCGUGAGCAAGAA
GGGCCUGCAGAAGAUCCUGAACGUGCAGAAGAAGCUGCCCAUCAUCCAG
AAGAUCAUCAUCAUGGACAGCAAGACCGACUACCAGGGCUUCCAGAGCA
UGUACACCUUCGUGACCAGCCACCUGCCCCCCGGCUUCAACGAGUACGA
CUUCGUGCCCGAGAGCUUCGACCGGGACAAGACCAUCGCCCUGAUCAUG
AACAGCAGCGGCAGCACCGGCCUGCCCAAGGGCGUGGCCCUGCCCCACC
GGACCGCCUGCGUGCGGUUCAGCCACGCCCGGGACCCCAUCUUCGGCAA
CCAGAUCAUCCCCGACACCGCCAUCCUGAGCGUGGUGCCCUUCCACCAC
GGCUUCGGCAUGUUCACCACCCUGGGCUACCUGAUCUGCGGCUUCCGGG
UGGUGCUGAUGUACCGGUUCGAGGAGGAGCUGUUCCUGCGGAGCCUGCA
GGACUACAAGAUCCAGAGCGCCCUGCUGGUGCCCACCCUGUUCAGCUUC
UUCGCCAAGAGCACCCUGAUCGACAAGUACGACCUGAGCAACCUGCACG
AGAUCGCCAGCGGCGGCGCCCCCCUGAGCAAGGAGGUGGGCGAGGCCGU
GGCCAAGCGGUUCCACCUGCCCGGCAUCCGGCAGGGCUACGGCCUGACC
GAGACCACCAGCGCCAUCCUGAUCACCCCCGAGGGCGACGACAAGCCCG
GCGCCGUGGGCAAGGUGGUGCCCUUCUUCGAGGCCAAGGUGGUGGACCU
GGACACCGGCAAGACCCUGGGCGUGAACCAGCGGGGCGAGCUGUGCGUG
CGGGGCCCCAUGAUCAUGAGCGGCUACGUGAACAACCCCGAGGCCACCA
ACGCCCUGAUCGACAAGGACGGCUGGCUGCACAGCGGCGACAUCGCCUA
CUGGGACGAGGACGAGCACUUCUUCAUCGUGGACCGGCUGAAGAGCCUG
AUCAAGUACAAGGGCUACCAGGUGGCCCCCGCCGAGCUGGAGAGCAUCC
UGCUGCAGCACCCCAACAUCUUCGACGCCGGCGUGGCCGGCCUGCCCGA
CGACGACGCCGGCGAGCUGCCCGCCGCCGUGGUGGUGCUGGAGCACGGC
AAGACCAUGACCGAGAAGGAGAUCGUGGACUACGUGGCCAGCCAGGUGA
CCACCGCCAAGAAGCUGCGGGGCGGCGUGGUGUUCGUGGACGAGGUGCC
CAAGGGCCUGACCGGCAAGCUGGACGCCCGGAAGAUCCGGGAGAUCCUG
AUCAAGGCCAAGAAGGGCGGCAAGAUCGCCGUGUGA

The design of single dose intravenous delivery of the BacLC/mRNA formulations and evaluation is shown in Scheme 5.

TABLE 9
Bacterial lipid compositions with luciferase mRNA

| | Ionizable:Structural or | | | | | | |
|---|---|---|---|---|---|---|---|
| | natural lipid:Sterol:PEG- | Ionizable | Structural or | | PEG- | mRNA | |
| Formulation | lipid Molar ratio | lipid | natural lipid | Sterol | lipid | Cargo | N:P |
| BacLC E. | 35:50:12.5:2.5 | C12-200 | Lipids extracted | Chol | DMPE- | FLuc:EPO | 15:1 |
| coli (Avanti) | | | from E.coli (Avanti) | | PEG2k | | |
| BacLC E. coli | 35:50:12.5:2.5 | C12-200 | Lipids extracted | Chol | DMPE- | FLuc:EPO | |
| | | | from E. coli | | PEG2k | | |
| BacLC | 35:50:12.5:2.5 | C12-200 | Lipids extracted | Chol | DMPE- | FLuc:EPO | |
| Salmonella | | | from Salmonella | | PEG2k | | |

As shown in Scheme 5, two mice were evaluated for each formulation. The test samples were injected intravenously into mice at 0 h, and then 4-6 h later mice were injected with D-Luciferin (200 μL, 15 mg/mL). After 5 minutes, animals were live imaged for whole body at auto exposure. Animals were then euthanized, and organs were imaged at 1 sec and 1 min exposures.

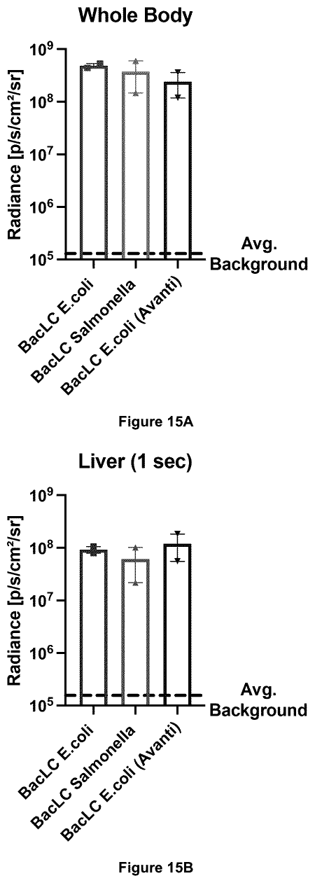

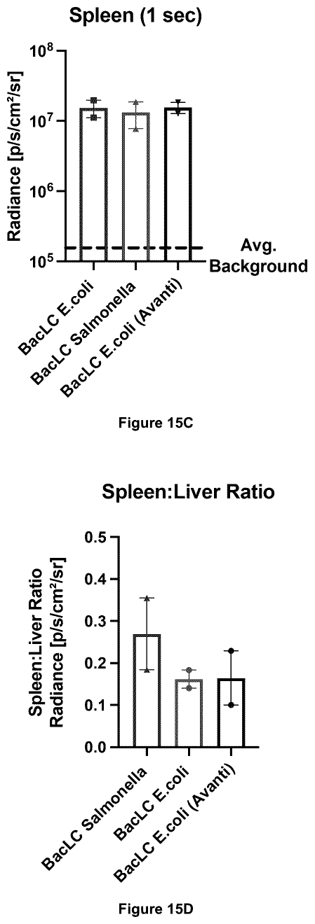

FIG. 15A-C show the whole body, liver, and spleen radiance of the mice 4-6 hours after the mice were intravenously administered with a dosage of BacLC E. coli/mRNA (E. coli BacLC, containing FLuc:EPO mRNA 10 μg), BacLC E. coli (Avanti)/mRNA (E. coli (Avanti) BacLC, containing FLuc:EPO mRNA 10 μg), and BacLC Salmonella/mRNA (Salmonella BacLC, containing FLuc:EPO mRNA 10 μg), prepared according to Example 5. FIG. 15D shows the spleen to liver ratio of radiance 4-6 hours after the mice were intravenously administered with a dosage of BacLC E. coli/mRNA (E. coli BacLC, containing FLuc:EPO mRNA 10 μg), BacLC E. coli (Avanti)/mRNA (E. coli (Avanti) BacLC, containing FLuc:EPO mRNA 10 μg), and BacLC Salmonella/mRNA (Salmonella BacLC, containing FLuc:EPO mRNA 10 μg). Results indicate all three BacLC show distribution to the liver and spleen, with BacLC Salmonella having a higher spleen:liver ratio in comparison.

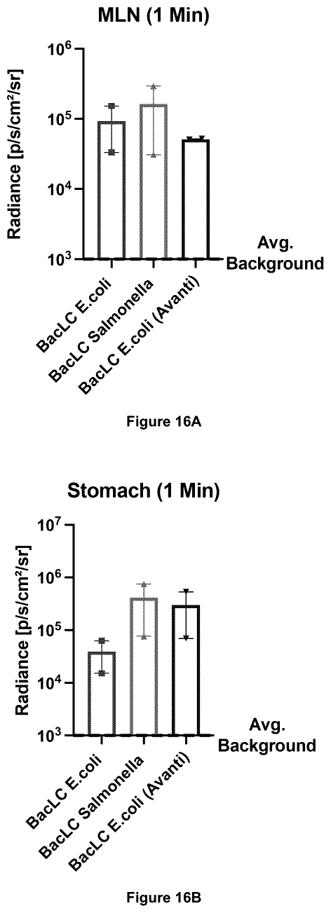

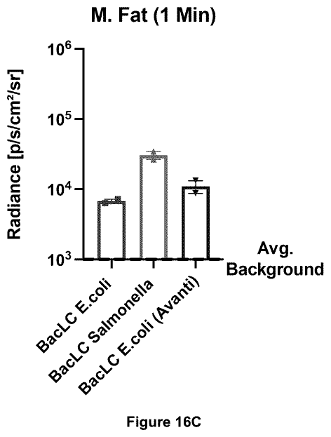

FIG. 16A-C show the mesenteric lymph node, stomach, and mesenteric fat pad radiance of the mice 4-6 hours after the mice were intravenously administered with a dosage of BacLC E. coli/mRNA (E. coli BacLC, containing FLuc:EPO mRNA 10 μg), BacLC E. coli (Avanti)/mRNA (E. coli (Avanti) BacLC, containing FLuc:EPO mRNA 10 μg), and BacLC Salmonella/mRNA (Salmonella BacLC, containing FLuc:EPO mRNA 10 μg), prepared according to Example 5. Results indicate all three BacLC formulations distributed to lymph nodes, stomach, and fat, with BacLC E. coli (Avanti) and BacLC Salmonella showing higher signal in the stomach compared to BacLC E. coli. All three had equivalent radiance in the lymph nodes.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference. Other embodiments are within the claims.

1. A bacteria-derived lipid composition, comprising:
(a) a bacterial component comprising one or more lipids extracted from a bacterial source; and
(b) an ionizable lipid.

2. The bacteria-derived lipid composition of claim 1, wherein the bacterial component comprises isolated bacterial extracellular vesicles

3. The bacteria-derived lipid composition of claim 1, wherein the bacterial component is modified by reconstructing a film comprising the bacterial component in the presence of the ionizable lipid.

4. (canceled)

5. The bacteria-derived lipid composition of claim 1, wherein the ionizable lipid has one or more characteristics selected from the group consisting of:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10.

6. The bacteria-derived lipid composition of claim 1, wherein the bacteria-derived lipid composition further comprises a sterol and a polyethylene glycol (PEG)-lipid conjugate.

7-8. (canceled)

9. The bacteria-derived lipid composition of claim 6, wherein the bacteria-derived lipid composition comprises:
about 20 mol % to about 50 mol % of the ionizable lipid,
about 20 mol % to about 60 mol % of the bacterial component,
about 7 mol % to about 45 mol % of the sterol, and
about 0.5 mol % to about 3 mol % of the polyethylene glycol (PEG)-lipid conjugate.

10. (canceled)

11. The bacteria-derived lipid composition of claim 6, wherein the bacteria-derived lipid composition comprises ionizable lipid:bacterial lipids:sterol:PEG-lipid at a molar ratio of about 35:50:12.5:2.5 or about 35:20:42.5:2.5.

12. (canceled)

13. The bacteria-derived lipid composition of claim 1, wherein the ionizable lipid is selected from the group consisting of 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), MD1 (cKK-E12), OF2, EPC, ZA3-Ep10, TT3, LP01, 5A2-SC8, Lipid 5, SM-102 (Lipid H), and ALC-315.

14. The bacteria-derived lipid composition of claim 1, wherein the ionizable lipid 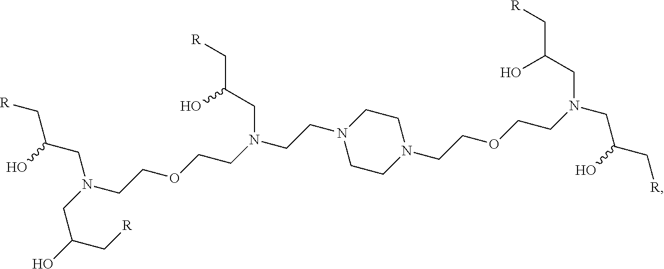
wherein each R is independently a C₈-C₁₄ alkyl group.

15. The bacteria-derived lipid composition of claim 1, wherein the bacterial source is selected from Escherichia, Acinetobacter, Agrobacterium, Anabaena, Aquifex, Azoarcus, Azotobacter, Bordetella, Bradyrhizobium, Brucella, Buchnera, Burkholderia, Candidatus, Chromobacterium, Crocosphaera, Dechloromonas, Desulfitobacterium, Desulfotalea, Erwinia, Francisella, Fusobacterium, Gloeobacter, Gluconobacter, Helicobacter, Legionella, Magnetospirillum, Mesorhizobium, Methylococcus, Neisseria, Nitrosomonas, Nostoc, Photobacterium, Photorhabdus, Polaromonas, Prochlorococcus, Pseudomonas, Psychrobacter, Ralstonia, Rubrivivax, Salmonella, Shewanella, Shigella, Sinorhizobium, Synechococcus, Synechocystis, Thermosynechococcus, Thermotoga, Thermus, Thiobacillus, Trichodesmium, Vibrio, Wigglesworthia, Wolinella, Xanthomonas, Xylella, Yersinia, Bacillus, Clostridium, Deinococcus, Exiguobacterium, Geobacillus, Lactobacillus, Lactobacillus, Moorella, Oceanobacillus, Symbiobacterium, and Thermoanaerobacterium.

16. The bacteria-derived lipid composition of claim 15, wherein the bacterial source is E. coli or Salmonella typhimurium.

17. The bacteria-derived lipid composition of claim 1, wherein the bacteria-derived lipid composition is a lipophilic moiety selected from the group consisting of a lipoplex, a liposome, a lipid nanoparticle, a polymer-based carrier, an exosome, a lamellar body, a micelle, and an emulsion.

18. (canceled)

19. The bacteria-derived lipid composition of claim 1, wherein the bacteria-derived lipid composition is a lipid nanoparticle having a size of less than about 200 nm.

20-23. (canceled)

24. The bacteria-derived lipid composition of claim 19, wherein the average polydispersity index (PDI) of the lipid nanoparticle ranges from about 0.1 to about 0.4.

25. (canceled)

26. The bacteria-derived lipid composition of claim 1, wherein the bacteria-derived lipid composition further comprises one or more heterologous functional agents, optionally encapsulated by, embedded on the surface of, or conjugated to the surface of the bacteria-derived lipid composition.

27. (canceled)

28. The bacteria-derived lipid composition of claim 26, wherein the heterologous functional agent comprises a polynucleotide.

29. The bacteria-derived lipid composition of claim 28, wherein the polynucleotide is mRNA.

30. The bacteria-derived lipid composition of claim 26, wherein the bacterial derived lipid nanoparticle has a total lipid:heterologous functional agent weight ratio of about 50:1 to about 10:1.

31-34. (canceled)

35. The bacteria-derived lipid composition of any one of the proceeding claims, further comprising a HEPES or TRIS buffer at a pH of about 7.0 to about 8.5.

36-37. (canceled)

38. The bacteria-derived lipid composition of claim 1, further comprising one or more cryoprotectants.

39-40. (canceled)

41. The bacteria-derived lipid composition of claim 1, wherein the bacteria-derived lipid composition is a lyophilized composition.

42. The bacteria-derived lipid composition of claim 41, wherein the lyophilized bacteria-derived lipid composition comprises one or more lyoprotectants.

43-52. (canceled)

53. A method for making a bacteria-derived lipid composition, comprising:
reconstructing (a) a bacteria component comprising one or more lipids extracted from a bacterial source in the presence of (b) an ionizable lipid, to produce the bacteria-derived lipid composition, wherein the ionizable lipid has two or more of the characteristics listed below:
(i) at least 2 ionizable amines;
(ii) at least 3 lipid tails, wherein each of the lipid tails is at least 6 carbon atoms in length;
(iii) a pKa of about 4.5 to about 7.5;
(iv) an ionizable amine and a heteroorganic group separated by a chain of at least two atoms; and
(v) an N:P ratio of at least 10, and
loading into the bacteria-derived lipid composition with one or more heterologous functional agents.

54-56. (canceled)


SEQUENCE LISTING (8 sequences)

SEQ ID NO: 1 - RNA, 200 nt, synthetic construct
  source 1..200: mol_type = other RNA
  misc_difference 1..200: note = This sequence may encompass 100-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 11 nt, synthetic construct
  source 1..11: mol_type = other RNA
gggatcctacc

SEQ ID NO: 3 - RNA, 250 nt, synthetic construct
  source 1..250: mol_type = other RNA
  misc_difference 1..250: note = This sequence may encompass 50-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 30 nt, synthetic construct
  source 1..30: mol_type = other RNA
aaaaaaaaaaaaaaaaaaaaaaaaaaaaaa

SEQ ID NO: 5 - RNA, 70 nt, synthetic construct
  source 1..70: mol_type = other RNA
aaaaaaaaaaaaaaaaaaaaaaaaaaaaaaaaaaaaaaaaaaaaaaaaaaaaaaaaaaaaaaaaaaaaaa

SEQ ID NO: 6 - RNA, 4308 nt, synthetic construct
  source 1..4308: mol_type = other RNA
taatacgactcactataaggagaataaactagtattcttctggtccccacagactcagagagaacccgccaccatgttcgtgttcctggtgctgctgcctctggtgtccagccagtgtgtgaacctgaccaccagaacacagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctgggcgtctactaccacaagaacaacaagagctggatggaaagcgagttccgggtgtacagcagcgccaacaactgcaccttcgagtacgtgtcccagcctttcctgatggacctggaaggcaagcagggcaacttcaagaacctgcgcgagttcgtgtttaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaaccttcctgctgaagtacaacgagaacggcaccatcaccgacgccgtggattgtgctctggatcctctgagcgagacaaagtgcaccctgaagtccttcaccgtggaaaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattacctgtaccggctgttccggaagtccaatctgaagcccttcgagcgggacatctccaccgagatctatcaggccggcagcaccccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaaaagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccaggacgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggaatctgcgccagctaccagacacagacaaacagccctcggagagccagaagcgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagcttcatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagcaggcgccgctctgcagatcccctttgctatgcagatggcctaccggttcaacggcatcggagtgacccagaatgtgctgtacgagaaccagaagctgatcgccaaccagttcaacagcgccatcggcaagatccaggacagcctgagcagcacagcaagcgccctgggaaagctgcaggacgtggtcaaccagaatgcccaggcactgaacaccctggtcaagcagctgtcctccaacttcggcgccatcagctctgtgctgaacgatatcctgagcagactggaccctcctgaggccgaggtgcagatcgacagactgatcacaggcagactgcagagcctccagacatacgtgacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatatgtgcccgctcaagagaagaatttcaccaccgctccagccatctgccacgacggcaaagcccactttcctagagaaggcgtgttcgtgtccaacggcacccattggttcgtgacacagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggacaagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccctggtacatctggctgggctttatcgccggactgattgccatcgtgatggtcacaatcatgctgtgttgcatgaccagctgctgtagctgcctgaagggctgttgtagctgtggcagctgctgcaagttcgacgaggacgattctgagcccgtgctgaagggcgtgaaactgcactacacatgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaacggct

SEQ ID NO: 7 - RNA, 1110 nt, synthetic construct
  source 1..1110: mol_type = other RNA
taatacgactcactataaggggaaataagagagaaaagaagagtaagaagaaatataagaccccggcgccgccaccatgaaggtgaaactcctagtgctgctgtgcaccttcacagcaacctacgccgacaccatctgcattggttatcacgcgaataatagtaccgacactgtcgacacagttctggagaagaatgtaaccgtgacccactccgtcaatttattggagaacgggggcgggggcaaatatgtttgtagcgccaaactgcgcatggtcactggattgaggaacaagccctccaagcagtcccaaggcctgttcggggctattgcaggttttacggagggcgggtggacagggatggtggatggttggtacgggtaccaccaccagaatgaacaaggatctggatacgccgccgatcagaagtctactcagaatgctatcaatggaatcaccaataaagttaattcagtgatagaaaaaatgaacacacagtacactgcaatcggctgcgaatataacaagtcagagagatgcatgaagcagatcgaggataagatcgaggagattgagtccaaaatatggtgttacaacgccgaactcctcgtactgctcgaaaacgagaggacacttgattttcatgacagtaacgtgaaaaacctgtatgaaaaagtgaaaagccagctaaagaacaacgctaaagaaattggcaatggatgttttgagttctaccataaatgcaatgatgagtgtatggagagcgtcaagaacggcacgtatgactacccaaagtatagcgaagaatcgaagctgaaccgggagaagatcgacggtgtcaagcttgagtctatgggcgtgtaccaaattgaaggacgatgataataggctggagcctcggtggcctagcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaagagc

SEQ ID NO: 8 - RNA, 1653 nt, synthetic construct
  source 1..1653: mol_type = other RNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga